(19) United States
(12) Patent Application Publication
(10) Pub. No.: US 2014/0294849 A1
(43) Pub. Date: Oct. 2, 2014

(54) COMPOSITIONS AND USES

(71) Applicants: Daniel Larocque, Laval (CA); Remi Palmantier, Rixensart (BE); Pascale Tribout-Jover, Laval (CA)

(72) Inventors: Daniel Larocque, Laval (CA); Remi Palmantier, Rixensart (BE); Pascale Tribout-Jover, Laval (CA)

(73) Assignee: GLAXOSMITHKLINE BIOLOGICALS SA, Rixensart (BE)

(21) Appl. No.: 14/236,747

(22) PCT Filed: Jan. 5, 2012
(86) PCT No.: PCT/EP2012/057963

§ 371 Date: Apr. 24, 2014

(30) Foreign Application Priority Data
Aug. 8, 2011 (GB) 1113570.4

(51) Int. Cl. A61K 39/39; G01N 33/68; A61K 39/395

(52) U.S. Cl. CPC A61K 39/39; A61K 39/3955; G01N 33/6896; A61K 2039/55572
USPC 424/139.1; 435/29; 435/7.24

(57) ABSTRACT

The invention provides a TLR agonist and an antibody which binds to an antigen such as a β-amyloid antigen for use in stimulating an immune response to the antigen in an individual. The TLR agonist can be delivered at the same time as the antibody, less than 48 hours before the antibody or after the antibody. The compositions and methods provided are useful for prevention or treatment of Alzheimer's disease.

### TECHNICAL FIELD

The present invention relates to compositions and methods for stimulation of the immune system. In particular, the present invention relates to methods of preventing or reducing amyloid deposition in a subject and compositions for use in such methods.

### BACKGROUND

Many approaches to therapy involve delivery of antigens, optionally with adjuvants, to provoke an immune response, such as an antibody response. Such delivery of antigens is often referred to as active immunisation.

Passive therapy can also be delivered, by administration of antibodies.

Both active and passive immunisation approaches have been considered in the treatment of Alzheimer's and other amyloidogenic diseases by administration of amyloid beta or other antigen or an antibody to such antigen to a patient under conditions that generate a beneficial immune response in the patient. The deposition of amyloid-beta (Abeta or Aβ or amyloid-β or β-amyloid or beta amyloid herein) peptides in the central nervous system in the form of amyloid plaques is one of the hallmarks of AD (U.S. Patent Publication No. 20040214774 to Wisniewski et al; U.S. Pat. No. 6,114,133 to Seubert; Wegiel et al., “Alzheimer Dementia Neuropathology,” in Dementia: Presentations, Differential Diagnosis & Nosology, 89-120 (Emery & Oxman, eds., 2003). Several lines of evidence favour the conclusion that amyloid beta accumulation destroys neurons in the brain, leading to deficits in cognitive abilities. Because accumulation of amyloid beta appears to be the result of a shift in equilibrium from clearance toward deposition, identifying and promoting mechanisms that enhance Aβ clearance from the brain is highly desirable. Other proteins are known that cause neurodegenerative diseases, such as Parkinson's disease, by the formation of plaques and neurofibrillary tangles.

Immunization of young PDAPP mice (which express a disease linked isoform of the human amyloid precursor protein (APP)) with synthetic human Aβ1-42 has been shown to reduce the extent and progression of AD-like neuropathologies (Schenk D. et al, 1999 Nature 400:173-7). One possible mechanism of action for the activity of this vaccine is that anti-Aβ antibodies facilitate clearance of amyloid-β, either before deposition or after plaque formation. A vaccine containing a pre-aggregated preparation of synthetic human Aβ42 peptides combined with QS21 (with or without polysorbate) was tested in clinical trials on patients with Alzheimer's disease (clinical trial AN1792). The phase 2 of this trial was halted due to the occurrence of sterile subacute meningoencephalitis in 18 out of 300 patients in the treatment group (Orgogozo J. M. et al. Neurology 2003; 61: 46-54). It has been proposed that the cause of the meningo encephalitis was due to the helper T cell of type 1 (TH1), which are pro-inflammatory. That immune response has been postulated to be triggered by the long Aβ42 peptide that contains T cell epitopes within amino acids 15 to 42 (Monsonego, A. et al. J. Clin. Invest. 2003; 112: 415-422).

There is still a need for optimized delivery and treatment regimens for prevention and treatment of disease.

### SUMMARY OF THE INVENTION

An aspect of the invention is a composition comprising a combination of an adjuvant and an antigen suitable for treatment or prevention of Alzheimer's disease, and/or for stimulating uptake of beta amyloid, and/or preventing or reducing amyloid deposition. A composition comprising a combination of an adjuvant and an antigen for use in the treatment or prevention of Alzheimer's disease or for stimulating uptake of beta amyloid and/or preventing or reducing amyloid deposition.

A further aspect of the invention is a composition comprising a combination of an adjuvant and an antigen for use in the preparation of a medicament for treatment or prevention of Alzheimer's disease or for stimulating uptake of beta amyloid and/or preventing or reducing amyloid deposition.

A further aspect of the invention is a method for the treatment or prevention of Alzheimer's disease, or for stimulating phagocytosis of beta amyloid, and/or preventing or reducing amyloid deposition, the method comprising delivery of an effective amount of a composition disclosed herein.

A further aspect of the invention is a Toll Like Receptor (TLR) agonist and an antigen, for use in stimulating an immune response to the antigen in an individual, wherein the TLR agonist and antigen are delivered separately.

A further aspect of the invention is the use of a TLR agonist and an antigen in the preparation of a medicament for the prevention and treatment of disease associated with the antigen, wherein the TLR agonist and antigen are delivered separately.

A further aspect of the invention is a composition for treatment or prevention of Alzheimer's disease, the composition comprising an adjuvant (such as a TLR agonist) and an antigen. The TLR agonist may comprise an aminoalkyl glucosaminide phosphate (AGP) and/or 3-O-desacyl-4-monophosphoryl lipid A (3D MPL), optionally and/or QS21, optionally with liposomes (suitably the GSK adjuvant AS01B).

A further aspect of the invention is a kit comprising a TLR agonist and an antigen and instructions for separate delivery of the TLR agonist and the antigen, for stimulating an immune response to the antigen in an individual.

A further aspect of the invention is a kit comprising an adjuvant, such as a TLR agonist, and an antigen for simultaneous or substantially simultaneous delivery for stimulating an immune response to the antigen in an individual, the kit being for use, or suitable for use, in treatment or prevention of Alzheimer's disease.

A further aspect of the invention is a composition comprising a combination of an adjuvant (such as a TLR agonist) and an antigen suitable for treatment or prevention of macular degeneration, Parkinson's disease, islet amyloid deposits in pancreas, ALS or Huntington's disease, and/or for stimulating uptake of beta amyloid, and/or preventing or reducing amyloid deposition.

A further aspect of the invention is a composition comprising a combination of an adjuvant (such as a TLR agonist) and an antigen for use in the treatment or prevention of macular degeneration, Parkinson's disease, islet amyloid deposits in pancreas, ALS or Huntington's disease or for stimulating uptake of beta amyloid and/or preventing or reducing amyloid deposition.

A further aspect of the invention is a composition comprising a combination of an adjuvant (such as a TLR agonist) and an antigen for use in the preparation of a medicament for treatment or prevention of macular degeneration, Parkinson's disease, islet amyloid deposits in pancreas, ALS or Huntington's disease or for stimulating uptake of beta amyloid and/or preventing or reducing amyloid deposition.

A further aspect of the invention is a method for the treatment or prevention of macular degeneration, Parkinson's disease, islet amyloid deposits in pancreas, Amyotrophic Lateral Sclerosis (ALS) or Huntington's disease, or for stimulating phagocytosis of beta amyloid, and/or preventing or reducing amyloid deposition, the method comprising delivery of an effective amount of a composition comprising a combination of an adjuvant (such as a TLR agonist) and an antigen.

A further aspect of the invention is a composition comprising a β-amyloid antigen and QS21 formulated in a liposome comprising a sterol. Such composition may be for use in preventing and/or treating Alzheimer's disease in a subject. A further aspect of the invention is the use of a composition comprising a β-amyloid antigen and QS21 formulated in a liposome comprising a sterol in the manufacture of a medicament for preventing and/or treating Alzheimer's disease in a subject.

A further aspect of the invention is the use a method of preventing and/or reducing amyloid deposition in a subject comprising treatment of a subject with an effective amount of a composition consisting or consisting essentially of QS21 formulated in a liposome comprising a sterol.

Further aspects of the invention include a composition consisting or consisting essentially of QS21 formulated in a liposome comprising a sterol for use in preventing and/or reducing amyloid deposition in a subject;

use of a composition consisting or consisting essentially of QS21 formulated in a liposome comprising a sterol in the manufacture of a medicament for preventing and/or reducing amyloid deposition in a subject; a method of preventing and/or treating Alzheimer's disease, macular degeneration, Parkinson's disease, islet amyloid deposits in pancreas, ALS or Huntington's disease in a subject comprising treatment of a subject with an effective amount of a composition consisting or consisting essentially of QS21 formulated in a liposome comprising a sterol;
a composition consisting or consisting essentially of QS21 formulated in a liposome comprising a sterol for use in preventing and/or reducing treating Alzheimer's disease, macular degeneration, Parkinson's disease, islet amyloid deposits in pancreas, ALS or Huntington's disease;
use of a composition consisting or consisting essentially of QS21 formulated in a liposome comprising a sterol for preventing and/or treating Alzheimer's disease, macular degeneration, Parkinson's disease, islet amyloid deposits in pancreas, ALS or Huntington's disease; and
a kit comprising a composition consisting or consisting essentially of QS21 formulated in a liposome comprising a sterol.

### BRIEF DESCRIPTION OF THE FIGURES

FIG. 1: Higher Aβ42 specific Immunogenicity promoted by TLR4 containing adjuvants such as AS01B compared to water-oil emulsion (AS03) when adjuvants are combined with the Aβ1-6 CRM conjugate.

FIG. 2 (A and B): Dose-response study showing different level of Aβ42-specific immunogenicity promoted by TLR containing adjuvants such as MPL or CRX601 (TLR4 ligands), AS15 (a TLR4 and TLR9 ligand) compared to appropriate controls (non adjuvanted Aβ40/42 alone at ratio 4:1), non adjuvanted Aβ1-6CRM alone or with water-oil emulsion (AS03) or in combination with AS03-CRX601.

FIG. 3: The number of monocytes is up-regulated 4.5 fold by AS01B adjuvant.

FIG. 4: Ex-vivo Aβ42 uptake by mouse monocytes is promoted after injection of MPL containing adjuvant (AS01B) by the addition of a specific AβmAb (2E7).

FIG. 5: Aβ uptake in peripheral blood per monocyte cell (MFI) is promoted by an N-terminus (Aβ1-7) Aβ-specific monoclonal antibody (2E7) on adjuvanted peripheral blood (adjuvant used: AS01B) compared to non-adjuvanted blood (PBS).

FIG. 6: Aβ uptake by peripheral blood per monocytes cell (MFI) is promoted by an anti-Aβ(1-6) sera.

FIG. 7: In vivo Aβ uptake in the peripheral blood is promoted following the injection of a TLR-containing Aβ1-6 CRM conjugate compared to negative control or passive immunization only.

FIG. 8: An increase in in vitro rapid uptake of Aβ42 by fresh monocytes from human peripheral blood following in vitro stimulation (24 hr) by two doses of AS01B.

FIG. 9: A flow cytometry gating protocol showing that there are an increased number of monocytes following a single injection of TLR4-containing adjuvants.

FIG. 10: Peripheral blood monocyte number following a single intramuscular injection of different doses of 3D MPL (5 μg, 25 μg and 50 μg).

FIG. 11: Peripheral blood monocyte number following a single intramuscular injection of different doses of AS01B ( 1/20 vs ⅕ vs mouse full dose).

FIG. 12: Peripheral blood monocyte number 24 hrs following a single intramuscular injection of different doses of CRX601 (0.2 μg to 20 μg).

FIG. 13: Peripheral blood monocyte number following a single intramuscular injection of different doses of CRX601 (0.2 μg and 2 μg) in combination of constant dose of AS03.

FIG. 14: Aβ42 ex vivo uptake by peripheral blood monocytes from mice pre-injected with TLR4-containing adjuvants such as AS01B (MPL 5 ug per mouse) or a synthetic TLR4 (CRX601, 2 ug dose).

FIG. 15: Results for Aβ total plaque loading in a therapeutic setting

FIG. 16: Aβ specific antibodies stimulate the uptake of soluble Aβ1-42 toxic peptides in an actin polymerization-dependent mechanism.

FIG. 17: Aβ specific antibody stimulates the uptake of Aβ1-42 by BV2 microglia, a process leading to the degradation of this toxic peptide.

FIG. 18: Phagocytosis of Aβ 1-42 peptide by the human microglial cell line CHME was observed after the addition of TLR2 containing adjuvants.

FIG. 19: QS21+liposome stimulates in vivo an increase in monocyte number (panel A) and monocyte activation state (Ly6C high) (panel B) after 24 hrs in the C57BL/6 mouse peripheral blood following intra muscular injection.

FIG. 20: QS21+liposome stimulates the ex vivo Aβ uptake by mouse peripheral blood monocytes after 24 hrs of the intra muscular injection in the C57BL/6 mouse.

FIG. 21: QS21+liposome+Aβ1-6 CRM197 peptide-carrier triggers higher anti Aβ immunogenicity compared to QS21+Aβ1-6CRM197 formulation. Graph plot including individual mouse data and standard deviation (SD). Multiple comparison tests denote a significant (P value less than 1%) improvement of Aβ1-6 CRM197+QS21+liposome over the non-liposome formulation (Aβ1-6 CRM197+QS21).

FIG. 22: Adjuvant selection: Dose-range of AS01B or CRX601 for antibody-mediated Aβ peripheral uptake after a single injection (24 hrs time point)

FIG. 23: Aβ total plaque loading analyses.

FIG. 24: Twelve weekly injections of 3D-MPL or CRX527 or CRX601 or AS01B in APP/PS1 mouse model shows a spatial memory improvement compared to non treated mice.

FIG. 25: Passive avoidance retention test.

FIG. 26: 3D Histology of the brain Ab plaque following 3D MPL and LPS treated APP/PS1 mice.

FIG. 27 (A to C): Results of behavioural and plaque loading using TLR4 agonists free of endotoxin.

FIG. 28: Endogenous Aβ42 measurement in the peripheral blood after Immunization

FIG. 29: High Aβ42 specific immunogenicity promoted by TLR2 agonist containing adjuvants such as Pam3CysLip peptide fused with a model Aβ fragment (Aβ1-6).

FIG. 30: A TLR2 agonist containing formulation could improve the working memory in amyloid deposition model (TASTPM).

FIG. 31: The usage of the TLR2 agonist, i.e. Pam3CysLip Aβ1-6 peptide, improves the survival rate of TASTPM mouse model.

FIG. 32: Evaluation of the impact of TLR4-containing adjuvants, i.e. AS01B and CRX601/AS03, on in vivo amyloid β1-42 uptake during and after vaccination inducing a polyclonal anti-Aβ1-42 antibody response.

FIG. 33: Vaccination with native Aβ peptides before the onset of Aβ deposition can down-modulate soluble amyloid (Aβ40 and Aβ42) in a mouse model containing APP and PS1 mutations

FIG. 34: AS01B+Aβ1-6CRM vaccine triggers a 5 fold higher antibody titer compared to aluminium hydroxide+Aβ1-6CRM vaccine (FIG. 34A). Both AS01B and antigen are needed to induce a robust capture and clearance of peripheral Abeta by the peripheral CD11b+ monocytes in vivo compared to Alum+Ag, AS01B alone or Ag alone (FIG. 34B).

### DETAILED DESCRIPTION

### Sequences

(SEQ ID NO: 1)
Aβ1-6: DAEFRH
(SEQ ID NO: 2)
Aβ2-7: AEFRHD
(SEQ ID NO: 3)
Aβ3-8: EFRHDS
(SEQ ID NO: 4)
Aβ3-8 pyr: pyrEFRHDS
(SEQ ID NO: 5)
Aβ11-16: EVHHQK
(SEQ ID NO: 6)
TYLIHVHIITIYHISIYYIVC
(SEQ ID NO: 7)
TYLIHVHIITIYHISIYYIV
(SEQ ID NO: 8)
SQEPAAPAAEAT PAAEAP
(SEQ ID No. 9)
SQEPAAPAAEATPAAEAPDAEFRH
(SEQ ID No. 10)
DMPVDPD
Aβ1-42:
(SEQ ID NO: 11)
DAEFRHDSGY EVHHQKLVFF AEDVGSNKGA IIGLMVGGVV IA
(SEQ ID NO: 12)
TCGTCGTTTTGTCGTTTTGTCGTT

The present invention relates to a number of different approaches to generate useful immune response, which include delivery of antibodies with an TLR agonist, delivery of an antigen with a TLR agonist (separately or in combination), and delivery of an antigen and adjuvant, where the adjuvant may be a TLR agonist but does not need to be a TLR agonist.

As used herein, ‘subject’ includes human subjects. As used herein, ‘individual’ includes human individuals.

In the present invention we demonstrate that Abeta specific antibodies can promote the metabolism of amyloid in peripheral blood. Specifically we demonstrate that uptake of labeled amyloid beta by CD11b+ monocytes is increased in the presence of a combination of an antibody specific to amyloid beta (specifically, 2E7) and the TLR4 agonist-containing Adjuvant System 01B (AS01B). We also demonstrate that certain compositions have the property of increasing uptake and have the property of increasing the number of cells capable of such amyloid beta uptake in the periphery.

In one embodiment the antibody and TLR agonist show a synergistic effect on phagocytic antigen uptake.

The phagocytic cells may be monocytes, the circulating precursors of macrophages, microglial cells or their precursors and dendritic cells, or any phagocytic cell as disclosed herein.

Reference to phagocytosis herein, and stimulation of phagocytosis herein, may be read more generally to include uptake of a material (e.g. an antigen) into a cell of the immune system. Likewise reference to cells capable of phagocytosis include reference to immune cells capable of antigen uptake, for example uptake of antigen bound to an antibody. As examples of uptake mechanisms, phagocytosis and macropinocytosis are specific examples, and the invention specifically contemplates both.

Reference to an antibody herein includes, in one aspect, reference to a full length antibody having both heavy and light chains. In another aspect reference to an antibody may be to a functional derivative or a fragment of an antibody mentioned herein, such as a Domain Antibody (dAb), single chain antibody, humanised antibody or chimaeric antibody.

In one aspect the functional derivative or fragment is able to stimulate phagocytosis, as disclosed herein, even in the absence of a TLR agonist. Stimulation of phagocytosis is suitably the phagocytosis of the protein to which the antibody binds. In one aspect a functional derivative or fragment of an antibody is able to bind an antigen and then to be recognised by cells of the immune system, suitably being able to be phagocytosed. In one aspect a functional derivative or fragment of an antibody is capable of synergistic interaction with a TLR agonist in the stimulation of phagocytosis, as disclosed herein.

In one aspect the antibody is a human or humanised antibody.

Antibodies can be raised against a polypeptide or portion of a polypeptide by methods known to those skilled in the art. Antibodies are readily raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. While both polyclonal and monoclonal antibodies can be used in the methods described herein, it is preferred that a monoclonal antibody is used where conditions require increased specificity for a particular protein.

An antibody against any antigen may be used. The antigen against which the antibody is raised or is specific to may be any suitable antigen, such as an antigen associated with disease, such as an antigen found in the human body, such as an amyloid protein. The antigen is suitably found in the central nervous system (CNS), peripheral nerves, optic nerves, or peripheral blood.

In one embodiment the antibody may be raised against, or be specific for a myelin protein, or fragment thereof, which may be used in the treatment of multiple sclerosis and spinal cord injury.

In one embodiment the antibody may be raised against, or be specific for an antigen located in the CNS.

In one embodiment the antibody may be raised against, or be specific for IAPP (islet amyloid polypeptide) Parkin, or huntingtin polyQ protein, or fragments of such antigens.

In one embodiment the antibody may be raised against, or be specific for an amyloid protein. As used herein, “amyloid” encompasses any insoluble fibrous protein aggregate that is deposited in the body. Amyloid deposition may be organ-specific (e.g. central nervous system, pancreas, etc.) or systemic. Amyloidogenic proteins thus include beta protein precursor, prion, [alpha]-synuclein, tau, ABri precursor protein, ADan precursor protein, amylin, apolipoprotein AI, apolipoprotein AII, lyzozyme, cystatin C, gelsolin, protein, atrial natriuretic factor, calcitonin, keratoepithelin, lactoferrin, immunoglobulin light chains, transthyretin, A amyloidosis, [beta]2-microglobulin, immunoglobulin heavy chains, fibrinogen alpha chains, prolactin, polyQ aggregates, keratin, and medin. Amyloid deposition may occur as its own entity or as a result of another illness (e.g. Down syndrome, amyloidosis, Parkinson's disease, Macular degeneration, glaucoma, multiple myeloma, chronic infection, diabetes or chronic inflammatory disease).

In one embodiment the antibody may be raised against, or be specific for, the amyloid beta protein. Amyloid beta is generated by the proteolysis of the Amyloid Precursor Protein (APP), and occurs in multiple isoforms, generally of 36-43 amino acids. The most common isoforms are believed to be Abeta(1-40) and Abeta(1-42). The sequence of Abeta(1-42) human is reported as: DAEFRHDSGY EVHHQKLVFF AEDVGSNKGA IIGLMVGGVV IA (SEQ ID NO:11). See, e.g., Roher et al., PNAS USA 90(22):10836-840 (1993). As used herein, amyloid-beta (or Abeta or Aβ or amyloid-β or β-amyloid or beta amyloid) includes human amyloid beta, including human amyloid beta (1-42) (SEQ ID NO:11).

In one embodiment the antigen is an N terminal fragment of beta amyloid, such as a fragment starting at amino acid position 1, 2, 3, 4, 5, 6, 7, 8, 9, 10, and ending at amino acid position 5, 6, 7, 8, 9, 10, 11, 12, 13, 14, 15, 16, 17, 18, 19, 20, suitably having a length of at least 5 amino acids, such as at least 6, or at least 7 or at least 8 amino acids. The fragment may comprise or consist of Abeta 1-6 and Abeta 3-8 or variants such as modified peptides, e.g. N-terminally pyroglutamated.

The antigen may be a C terminal Abeta fragment starting at amino acid position 1, 2, 3, 4, 5, 6, 7, 8, 9, 10 (counting from the C terminus), and ending at amino acid position 5, 6, 7, 8, 9, 10, 11, 12, 13, 14, 15, 16, 17, 18, 19, 20 (counting from the C terminus), suitably having a length of at least 5 amino acids, such as at least 6, or at least 7 or at least 8 amino acids.

Other suitable amyloid fragments are disclosed in WO 2000/072870; WO 2000/072880; WO 2002/096350; WO 2004/069182; WO 2006/121656; WO 2007/096076; WO 2007/068411.

One antibody for use in the invention is 6E10, a mouse monoclonal IgG₁, raised against amino acids 1-17 of β-Amyloid of human origin and available commercially.

Mouse antibodies may be humanised.

Another antibody for use in the invention is a monoclonal antibody 2E7 raised against Abeta 1-40. Abeta peptide 1-40 immunisations were carried out using conventional Freunds' adjuvant (CFA) protocols. The first immunisation of the AB1-40 was given subcutaneously in CFA, 100 ug total peptide. Subsequent injections were intraperitoneal (i.p.) in CFA, again 100 ug total peptide.

2E7 is disclosed in WO2007113172: VH SEQ ID NO:17; VL SEQ ID NO:20.

Other antibodies that may be used include humanised antibodies having one or more CDR's from antibody 2E7, such as all 6 CDRs from 2E7. CDRs are disclosed as SEQ ID NO:1-6 of WO2007113172.

An alternative antibody that may be used is the H2L1 humanised antibody disclosed in WO2007113172, (VH SEQ ID NO:28; VL SEQ ID NO:32; full heavy chain SEQ ID NO:36; full light chain SEQ ID NO:40).

In a further aspect the antibody may be the mouse 6F6 antibody, or a humanised version thereof, for example any such antibody disclosed in WO2009074583, for example H51L21, or an antibody having one or more CDRs, such as 6 CDRs, and/or one or more light or heavy of H51L21 (VH SEQ ID NO:65; VL SEQ ID NO:71. CDRS SEQ ID NO:1-6 of WO2009074583).

In one embodiment the antibody is an antibody specific for amyloid beta, or other amyloid polypeptide, and the TLR agonist is a TLR4 agonist.

In a further aspect of the invention a TLR agonist may be delivered to a subject in combination with an antigen, the antigen serving to generate antibodies.

Thus the invention relates to a method for stimulating an immune response in an individual, the method comprising delivering to an individual an antigen and, separately, a TLR agonist.

In one aspect the antigen that is delivered may be considered to be a means to generate an antibody to a host protein associated with disease.

The antigen may be a protein or a part thereof. The antigen may be associated with a disease state.

The antigen may be any antigen disclosed herein.

In one aspect the antigen is synuclein protein or fragment or variant thereof, or mutant having an addition, substitution or deletion, capable of raising an antibody response which recognizes synuclein or a fragment thereof, optionally conjugated to a protein, and/or optionally adjuvanted. The peptide may be alpha synuclein, and the antigen may comprise the sequence DMPVDPD, or part thereof. Alpha-synuclein may be a 140 aa protein represented by accession number NP_{—}000336.1 or GI:4507109 (obtainable from the ncbi.nlm.nih.gov website). Beta-synuclein may be a 134 aa protein represented by accession number NP_{—}001001502.1 or GI:48255903 (obtainable from the ncbi.nlm.nih.gov website).

In one embodiment antigens include amyloidogenic proteins, such as those listed above.

In one embodiment the antigens include amyloidogenic peptide Abeta1-42 or Abeta1-40.

Antigens and peptides, such as amyloid beta antigens and peptides, may be monomeric, oligomeric or aggregated.

The delivered antigen may be conjugated to a carrier, for example CRM (Cross-Reacting Material 197) or KLH (Keyhole Limpet Hemocyanin). When conjugated to a carrier, the antigen may contain an additional C-terminal cysteine residue, for conjugation. For example, an Aβ(fragment)-CRM antigen may contain the Aβ fragment, an additional Cysteine residue, and the CRM carrier protein.

By way of example, amyloid beta 1-6 conjugated to CRM or KLH may be used to raise antibodies to amyloid beta (full length or fragments thereof).

In one embodiment the antigens include any antigen mimicking Abeta 1-42 or fragments thereof, such that the antibody response induced to such antigen is also reactive with Amyloid beta.

In one embodiment of this aspect, the antigen is an N-terminal Abeta fragment, including from human Abeta, starting at amino acid position 1, 2, 3, 4, 5, 6, 7, 8, 9, 10, and ending at amino acid position 5, 6, 7, 8, 9, 10, 11, 12, 13, 14, 15, 16, 17, 18, 19, 20 suitably having a length of at least 5 amino acids, such as at least 6, or at least 7 or at least 8 amino acids. The fragment may comprise or consist of Amyloid beta 1-6 or amyloid beta 3-8 or variants, such as modified peptides, e.g. N-terminally pyroglutamated. The antigen may be a C terminal Abeta fragment, including from human Abeta, starting at amino acid position 1, 2, 3, 4, 5, 6, 7, 8, 9, 10 (counting from the C terminus), and ending at amino acid position 5, 6, 7, 8, 9, 10, 11, 12, 13, 14, 15, 16, 17, 18, 19, 20 (counting from the C terminus), suitably having a length of at least 5 amino acids, such as at least 6, or at least 7 or at least 8 amino acids.

Other suitable amyloid fragments are disclosed in WO 2000/072870; WO 2000/072880; WO 2002/096350; WO 2004/069182; WO 2006/121656; WO 2007/096076; WO 2007/068411.

In one embodiment the present invention relates to the use of a TLR agonist promoting the generation of and/or uptake activity of monocytes in combination with an immunogenic composition or vaccine creating an Aβ-specific antibody in vivo, suitably for treating neurodegenerative diseases such as Alzheimer's disease.

In another embodiment the antigen is suitably found in the central nervous system, peripheral nerves, optic nerves or peripheral blood.

In one embodiment the antigen may be a myelin protein, or fragment thereof, which may be used in the treatment of multiple sclerosis and spinal cord injury.

In one embodiment the antigen may be located in the CNS.

In one embodiment the antigen may be IAPP (islet amyloid polypeptide) Parkin, alpha or beta synuclein or Huntington polyQ protein, or fragments of such antigens.

In one aspect the antigen may be associated with macular degeneration, Parkinson's disease, islet amyloid deposits in pancreas, ALS or Huntington's disease.

The β-amyloid antigen may be any antigen known in the art that is suitable for raising a specific immune response against β-amyloid peptide. Suitably the antigen may be an immunogenic fragment of the β-amyloid peptide, including human beta amyloid peptide. The immunogenic fragment typically consists of 5 or more contiguous amino acids from the β-amyloid peptide sequence, in order to be of sufficient length to generate an antibody response. It may further be desirable to select an antigen that lacks an epitope that would generate a T-cell response, in order to avoid provoking an undesired inflammatory response. Since the N-terminus of the β-amyloid peptide (amino acids 1-15) does not contain a T cell epitope, the immunogenic fragment may suitably be selected from this region. Alternatively, since T cell epitopes are generally greater than 10 contiguous amino acids, the fragment may consist of 10 or fewer contiguous amino acids from the β-amyloid peptide sequence.

In one embodiment the antigen is or comprises an N-terminal fragment of beta amyloid, such as a fragment starting at amino acid position 1 and ending at amino acid position 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42 of the β-amyloid peptide sequence. In a particular embodiment the antigen may comprise or consist of Aβ1-5, Aβ1-6, Aβ1-7, Aβ1-10, Aβ1-14 or Aβ1-15.

In another embodiment, the antigen is or comprises an N-terminal truncated Aβ fragment, such as a fragment starting at amino acid position 2, 3, 4, 5, 6, 7, 8, 9, 10, 11, 12, 13 14, 15, 16, 17, 18, 19, 20, 21, 22, 23, 24, 25, 26, 27, 28, 29, 30, 31, 32, 33, 34, 35, 36, 37, 38, 39, 40 or 41 of the β-amyloid peptide sequence. The N-terminal truncated Aβ fragment may have a length of 2, 3, 4, 5, 6, 7, 8, 9, 10, 11, 12, 13 14, 15, 16, 17, 18, 19, 20, 21, 22, 23, 24, 25, 26, 27, 28, 29, 30, 31, 32, 33, 34, 35, 36, 37, 38, 39, 40 or 41 contiguous amino acids from the β-amyloid peptide sequence. In a particular embodiment the antigen may comprise or consist of 42-7, 42-8, 43-7, 43-8, 411-16 or 411-17.

The N-terminal truncated Aβ fragment may have a post-translational modification, such as methylation or pyroglutamation. For example, the N-terminal truncated Aβ fragment may have a pyroglutamate residue at its N-terminus, designated herein as p(E), pE, pyrE, or pyrE. In one aspect of the invention, the N-terminal pyroglutamate residue corresponds to position 3 or 11 of the full length Aβ sequence. In a particular embodiment, the antigen may comprise or consist of Aβp(E)3-7, Aβp(E)3-8, Aβp(E)3-40, Aβp(E)3-42, Aβp(E)11-16, Aβp(E)11-17, Aβp(E)11-40 or Aβp(E)11-42. Other suitable N-terminal truncated and/or post-translationally modified fragments are disclosed in WO 2004/013172.

The invention also includes combinations of Aβ antigens. The combination may consist of or comprise two different N-terminal fragments; an N-terminal fragment and an N-terminal truncated fragment; or two different N-terminal truncated fragments. Specifically contemplated combinations include: Aβ 1-6 and Aβp(E)3-8; Aβ1-6 and Aβ3-8; Aβ1-6 Aβ11-16; Aβ1-6 and Aβp(E)11-16; Aβp(E)3-8 and Aβp(E)11-16; Aβp(E)3-8 and 411-6; and Aβp(E)11-16 and Aβ3-8.

The above amyloid beta fragments and combinations are also suitable for use in the other aspects of the invention as disclosed below.

In one embodiment the antigen is any antigen disclosed in WO 2010011999, such as SEQ ID 10-13 from that publication. In one aspect the antigen comprises or consists of the following sequences, optionally conjugated to a carrier, such as CRM, or gold particles: TYLIHVHIITIYHISIYYIVC or TYLIHVHIITIYHISIYYIV. Conjugations may be at the Amine or carboxyl end.

In one aspect the antigen is a peptide able to trigger at least 0.1 ug per ml of specific antibody in the blood or serum when delivered in a suitable form to an individual in need of treatment.

In one aspect the antibody raised to the antigen is of the IgG1, IgG2, IgG3, and/or IgG4 isotypes.

In one embodiment the antibody and TLR agonist show a synergistic effect on phagocytotic antigen uptake.

The antigen is, in one aspect, delivered separately from the TLR agonist. In one aspect this separate delivery is delivery at a different time. The antigen and TLR agonist may be delivered by different routes, or provided in different formulations. Separate delivery of the antigen and TLR agonist may be achieved by delivery of an antigen, optionally formulated with an adjuvant (which may be a TLR agonist), at a different time to the delivery of a composition comprising a TLR agonist.

Separate delivery of the antigen and TLR agonist may also be achieved by delivery of an antigen not formulated with a TLR agonist at a different time to the delivery of a composition comprising a TLR agonist. In one embodiment, the antigen is not formulated with a TLR agonist may be delivered 1, 2, 3, 4, 5, 6 or more times, suitably up to 3 or 4 times, before the first delivery of the TLR agonist.

In one embodiment the TLR agonist may be delivered to an individual who has a pre-existing antibody population against the antigen when the TLR agonist is delivered. In one aspect the antibodies are those generated in response to the delivered antigen, i.e., not naturally occurring pre-existing antibodies. Antibody levels may be tested using conventional technologies, or may be assumed once an appropriate interval has elapsed after delivery of the means to generate the antibody, such that a majority of individuals have generated an antibody population specific for the antigen, such as more than 50%, more than 60%, more than 70% have detectable antibodies to the antigen, for example as detected by ELISA.

In one aspect the level of detectable antibodies is sufficient to display an effect on phagocytosis in the absence of a TLR4 agonist. In one aspect the level of detectable antibodies is sufficient to display an effect on phagocytosis in the presence of a TLR4 agonist, such as a synergistic effect.

Thus in one embodiment the TLR agonist is delivered after the antigen, which may for example be 1 week, 2 weeks, 3 weeks, a month, 5 weeks, 6 weeks, 7 weeks or 2 months after the first delivery of means to generate the antibody, or even more than 2 months, for example where antibody titers take this long to develop.

The antigen may be delivered 1, 2, 3, 4, 5, 6 or more times before the first delivery of the TLR agonist.

The TLR agonist may be delivered single or multiple times such as 1, 2, 3 or 4 times.

For example, the delivery regimen may comprise delivery of an antigen at day zero, followed by, if necessary, further delivery of antigen in week 1, 2, 3, or 4. Optionally delivery may be repeated with 1 or 2 or 3 or a 4 week intervals to induce an appropriate immune response, such as a suitable antibody response. A TLR agonist, alone or as part of a composition, may be delivered for example a week, 2 weeks, 3, weeks or 4 weeks after the initial antigen delivery, or 1, 2, 3, or 4 weeks after a second or subsequent antigen delivery.

Antigens for use in generating antibodies may be combined with an adjuvant to enhance the immune response against the antigen.

For the avoidance of doubt, reference to adjuvant herein is generally made in the context of the component delivered with an antigen to enhance the immunogenic response to that antigen, noting that TLR agonists themselves may be used as adjuvants with antigens, and thus TLR agonists may play a role both in the generation of antibodies, and following the delivery or generation of an antibody or other immune response.

The adjuvant used with the antigen, where necessary, and the adjuvant employed after generation of antibodies may be the same adjuvant.

The adjuvant for use with an antigen may be any suitable adjuvant which enhances the immune response against the antigen. The adjuvant may be a TLR agonist, such as a TLR1, TLR2, TLR 3, TLR 4, TLR 5, TLR 6, TLR 7, TLR 8, or TLR 9 agonist, such as a TLR2 or TLR4 or TLR9 agonist. The adjuvant may comprise a TLR agonist in combination with another adjuvant or pharmaceutically acceptable component.

In one embodiment the antigen is not combined with a TLR agonist.

In one embodiment the adjuvant helps to promote an antibody response, such as a TH2 adjuvant, such as alum.

In another embodiment the adjuvant is an oil-in-water emulsion.

In another embodiment the adjuvant comprises a water-oil emulsion such as AS03 in combination with an aminoalkyl glucosaminide phosphate such as CRX601.

In another embodiment the compositions of the invention may comprise an adjuvant containing MPL, QS21 and liposome. In a particular aspect, the compositions of the invention comprise the adjuvant system AS01B, which contains 50 μg MPL and 50 μg QS21 per human dose in a liposome formulation

An adjuvant for use in combination with an antigen may comprise 3D MPL and/or QS21, and/or a liposome, and may be AS01B.

AS01B is an Adjuvant System containing 3DMPL, QS21 and liposome (50 μg 3DMPL and 50 μg QS21 per human dose)

In one aspect the adjuvant is Trehalose MPL from Sigma, which comprises monophosphoryl lipid A (detoxified endotoxin) from S. minnesota (MPL) and synthetic trehalose dicorynomycolate (TDM) in 2% oil (squalene)-Tween 80-water.

In one embodiment the TLR agonist is AS01B, in a pharmaceutically acceptable form, and the antigen is amyloid beta, or part thereof, preferably comprising amino acids located within the N terminal 10 amino acids of amyloid beta, and wherein the AS01B is delivered to the individual after the antigen.

The antigen for use with adjuvant may comprise any one or more of an amyloid protein or fragment thereof disclosed in detail above.

For the avoidance of doubt the present invention also specifically relates to combinations of any adjuvant disclosed herein with any antigen as disclosed herein, which may be used as immunogenic compositions and vaccines. Such combinations may be used without the need for delivery of a separate TLR agonist.

Such antigen plus adjuvant combinations may be delivered in single or multiple doses. Such compositions may comprise a TLR agonist and an antigen fused or covalently linked for use in stimulating an immune response to the antigen in an individual, wherein the TLR agonist and antigen are delivered in the same formulation. In one embodiment the antigen plus adjuvant combination is delivered to a subject 1, 2, 3, 4, 5 or more times. The 2^{nd}, 3^{rd}, 4^{th} or subsequent delivery of the antigen plus adjuvant combination may act as a booster dose.

The compositions and methods of the various aspects of the invention may be used in the treatment or prevention of diseases or other conditions in subjects, including humans, who have a disease or condition or are at risk of developing the disease or condition.

Treatment can be therapeutic or preventative. The subject will be one who is in need of such treatment, including individuals already suffering from a particular medical disease or condition, as well as individuals who are at risk of developing the disease or condition. As used herein, the term “treatment” encompasses the alleviation, reduction, prevention, or delay of onset, of at least one aspect or symptom of a disease or condition in a subject (compared to a subject who does not receive such treatment).

As used herein, the term “effective amount” means that amount of a composition or pharmaceutical agent that will elicit the biological or medical response of a tissue, system, animal or human that is being sought, for instance, by a researcher or clinician. As used herein a therapeutically effective amount is an amount effective to ameliorate or reduce one or more aspects or symptoms of a disease or condition in a subject. A prophylactically effective amount is an amount which prevents or delays the onset of one or more aspects or symptoms of a disease described herein. An amount may have both therapeutic (ameliorating) and prophylactic (preventing or delaying) effects.

As used herein, the ability of a treatment to prevent or reduce amyloid deposition is as compared to amyloid deposition in a comparable subject who has not received the treatment. As used herein, the ability of a treatment to stimulate the uptake of beta amyloid is as compared to uptake in a comparable subject who has not received the treatment.

The methods described herein need not effect a complete cure or eradicate every symptom or manifestation of a disease or condition to constitute a useful treatment. As is recognised in the medical arts, drugs employed as therapeutic agents may reduce the severity of a given disease state, but need not abolish every manifestation of the disease to be regarded as useful therapeutic agents. Similarly, a prophylactically administered treatment need not be effective in preventing the onset of all aspects of a disease, or effective in all subjects treated, to constitute a viable prophylactic or preventative agent. Simply reducing the impact of a disease (for example, by reducing the number or severity of its symptoms in an individual, or reducing the number of subjects affected), is sufficient.

Thus the invention relates to a method of prevention or treatment of a disease associated with an antigen, the method comprising delivering to an individual:

1 a TLR agonist and an antibody which binds to that antigen; or
2 a TLR agonist and, separately, any antigen which generates an antibody to the antigen associated with disease; or
3 an adjuvant, which may be a TLR agonist, delivered in combination with an antigen which generates an antibody to the antigen associated with disease.

The invention also relates to a TLR agonist and an antibody which binds to an antigen for use in prevention or treatment of a disease associated with that antigen.

The invention also relates to a composition comprising an adjuvant and an antigen for use in prevention or treatment of a disease associated with that antigen.

The invention also relates to a TLR agonist and, separately, an antigen, for use in prevention or treatment of a disease associated with that antigen.

Such disease and conditions include, but are not limited to, Alzheimer's disease, diffuse Lewy body disease, Down syndrome, hereditary cerebral hemorrhage with amyloidosis, Creutzfeldt-Jakob disease, Gerstmann-Straussler-Scheinker disease, fatal familial insomnia, macular degeneration, glaucoma, British familial dementia, Danish familial dementia, familial corneal amyloidosis, Familial corneal dystrophies, medullary thyroid carcinoma, insulinoma, type 2 diabetes, isolated atrial amyloidosis, pituitary amyloidosis, aortic amyloidosis, plasma cell disorders, familial amyloidosis, senile cardiac amyloidosis, inflammation-associated amyloidosis, familial Mediterranean fever, dialysis-associated amyloidosis, systemic amyloidosis, and familial systemic amyloidosis.

Other conditions include ocular diseases, e.g. wet and dry age related Macular Degeneration (AMD), geographic atrophy, glaucoma, Abeta dependent cataract formation, as disclosed in WO2009040336 and WO 2009074583.

In one embodiment the invention relates to treatment or prevention of diseases associated with amyloid deposition, such as Alzheimer's disease.

The disease may be treated with the methods and compositions of the invention comprising an antibody, or an antigen generating an antibody, which antibody is specific to an antigen associated with that disease state. For example, for use in Alzheimer's disease, the invention contemplates the use of an antibody to amyloid beta, or the use of amyloid beta or fragment thereof, to raise an antibody response to amyloid beta.

The invention also relates to prevention or treatment of diseases wherein sequestering of an antigen in the periphery of an individual by antibody binding decreases a disease state.

In one embodiment the subject for prevention or treatment may have already been diagnosed with symptoms of a disease, for example a disease characterised by amyloid deposition. In one aspect the subject for treatment has not already been diagnosed with symptoms of a disease, for example a disease characterised by amyloid deposition.

The invention also relates to any composition disclosed herein for the treatment of diseases mentioned above, such as (antigen carrier), (antigen, adjuvant, carrier) and (antigen adjuvant) combinations, and to a method of treatment or prevention of disease using said compositions.

Preferred compositions include an antigen and adjuvant having:

as antigen, amyloid beta (N terminal) 1-6 or 3-8,
as adjuvant: a TLR 4 or TLR2 agonist, such as 3DMPL, or AS01B, or an AGP such as CRX601 or CRX527, or an AGP combined with an oil in water emulsion such as CRX601 with AS03, or a combination of a CpG and 3D MPL.

Compositions may have the amyloid beta peptide conjugated to a carrier such as CRM or KLH. Conjugation technologies are well known in the art. The Aβ antigen(s) may be conjugated to a carrier, typically a protein carrier, optionally via a linker. Suitable carrier molecules include CRM197, KLH, tetanus toxin, cholera toxin, viral like particles (VLP) and exoprotein A. Any suitable conjugation methodology may be used and such techniques are well known in the art. Where more than one Aβ antigen is used, each antigen may be conjugated to different carrier molecules or to the same carrier molecule. For example, each antigen may be conjugated to separate lots of the same carrier molecule and then mixed.

In one aspect, the composition of the invention comprises or consists essentially of AS01B and an antigen disclosed herein, such as amyloid beta (N terminal) 1-6 or 3-8, conjugated to CRM.

The invention also relates to compositions as described herein and the use of compositions for improved uptake, such as improved phagocytosis, of a desired target, for example phagocytosis of Amyloid beta.

Without wishing to be bound by theory, the use of a TLR agonist (such as an aminoalkyl glucosaminide phosphate, 3D-MPL or MPL or AS01B) is thought to stimulate of the innate immune system. Stimulation of the immune system may result in improved phagocytosis of antibodies which are bound to an antigen.

In one aspect the present invention relates to an effect on the deposits of amyloid protein, and in another aspect to an effect on behaviours that are associated with disease states, and in particular prevention or reduction of behaviours associated with Alzheimer's disease. In one aspect the methods and compositions of the invention have an effect both on amyloid protein deposition and behaviour associated with disease, such as behaviour associated with Alzheimer's disease, although in another aspect the methods and compositions of the invention have an effect either at the level of amyloid deposits or at the level of behaviour. In one aspect the prevention or reduction in severity of Alzheimer's disease comprises prevention or reduction of loss of memory. In a further aspect the invention relates to relates to improvement in memory. The memory may be spatial memory.

Amyloid beta in the brain is removed across the blood-brain-barrier by Low-density lipoprotein receptor-related protein-1 (LRP). LRP binds A-Beta in the brain and then transports it into the blood in a process called transcytosis. This process is in equilibrium with partitioning back into the brain. The peripheral sink hypothesis suggests that removal of the A-Beta from the blood shifts the equilibrium to bias it towards removal of Abeta from the brain (Nature Medicine 13, 1029-1031 (2007). Few mechanisms have been postulated such as clearance promoted by circulating lipoprotein receptors (Deane R, Sagare A, Zlokovic B V. The role of the cell surface LRP and soluble LRP in blood-brain barrier Abeta clearance in Alzheimer's disease. Curr Pharm Des. 2008; 14(16):1601-5).

Clearance of amyloid β by circulating lipoprotein receptors). The present invention shows that the use of a TLR agonist, in combination with an antibody is able to synergistically increase the phagocytosis of an antigen.

Thus in one aspect the invention relates to a method of preventing and/or reducing amyloid deposition or Alzheimer's disease in a subject comprising stimulating the innate immune system of an individual using the methods and compositions of the invention under conditions effective to prevent or reduce amyloid deposits, suitably by enhancing uptake, and optionally intracellular degradation, of the amyloid in cells of the immune system.

In both the active and passive approaches of the invention a TLR agonist may be, or is, used.

In one aspect the TLR agonist is a TLR1, TLR2, TLR 3, TLR 4, TLR 5, TLR 6, TLR 7, TLR 8 or TLR 9, agonist, such as a TLR2, TLR4 or TLR9 agonist.

In one aspect the TLR2 agonist is Pam3Cys—SQ EPAAPAAEAT PAAEAP.

The TLR agonist may be used alone, suitably formulated, or comprised within a composition with other components, such as other pharmaceutically active agents.

In one aspect the TLR agonist is a TLR4 agonist

In one aspect the TLR agonist is not a TLR9 agonist.

In one aspect the TLR agonist is not coupled to an antigen.

In one aspect the TLR agonist is not a TLR9 agonist coupled to an antigen.

Suitable TLR4 agonists include MPL and 3D-MPL, which are less toxic than Lipid A. Both are TLR4 agonists. U.S. Pat. No. 4,436,727 discloses monophosphoryl lipid A [MPL] and its manufacture. U.S. Pat. No. 4,912,094 and reexamination certificate U.S. Pat. No. 4,912,094 discloses 3-O-deacylated monophosphoryl lipid A [3D MPL] and a method for its manufacture, both of which are incorporated herein by reference.

In one aspect the TLR agonist or adjuvant is in the form of a pharmaceutical composition comprises 3D-MPL in combination with a saponin, such as QS21, and liposomes. In one aspect, the TLR agonist or adjuvant comprises QS21, and liposomes. In one aspect the composition consists or consists essentially of AS01B (see for example EP822831).

In one aspect the composition for use in the invention comprises a combination of a TLR4 agonist such as monophosphoryl lipid A, and a saponin derivative, particularly the combination of QS21 and 3D-MPL as disclosed in WO 94/00153, or a less reactogenic composition where the QS21 is quenched with cholesterol as disclosed in WO96/33739. An adjuvant formulation involving QS21 3D-MPL and tocopherol in an oil in water emulsion is described in WO95/17210 and is a suitable formulation.

In one aspect the TLR4 agonist may be a synthetic TLR4 agonist such as a synthetic disaccharide molecule, similar in structure to MPL and 3D-MPL or may be synthetic monosaccharide molecules, such as the aminoalkyl glucosaminide phosphate (AGP) compounds disclosed in, for example, WO9850399, WO0134617, WO0212258, WO3065806, WO04062599, WO06016997, WO0612425, WO03066065, and WO0190129 the disclosure of each of which is herein incorporated by reference. Such molecules have also been described in the scientific and patent literature as lipid A mimetics

The TLR4 agonist may be a lipid A mimetic. Lipid A mimetics suitably share some functional and/or structural activity with lipid A, and in one aspect are recognised by TLR4 receptors. AGPs as described herein are sometimes referred to as lipid A mimetics in the art. Lipid A mimetics in one aspect are less toxic than lipid A.

In one aspect the aminoalkyl glucosaminide phosphate (AGP) is one in which an aminoalkyl (aglycon) group is glycosidically linked to a 2-deoxy-2-amino-a-D-glucopyranose (glucosaminide) to form the basic structure of the claimed molecules. The compounds are phosphorylated at the 4 or 6 carbon on the glucosaminide ring. Further, the compounds possess three 3-alkanoyloxyalkanoyl residues comprising a primary and secondary fatty acyl chain, each carbon chain consisting of from 2-24 carbon atoms, and preferably from 7-16 carbon atoms. In one preferred aspect, each primary chain contains 14 carbon atoms and each secondary chain has between 10 and 14 carbon atoms.

In one aspect the AGP compounds are described by the general formula:

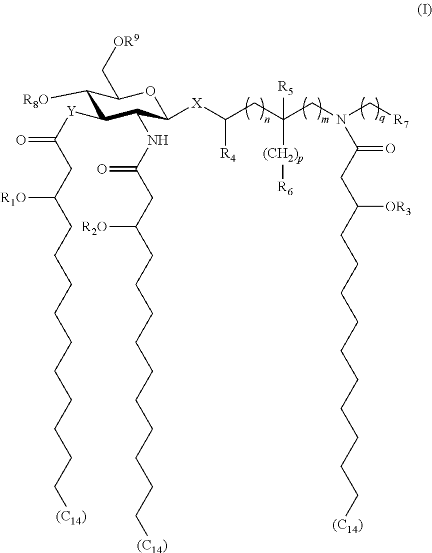

Such compounds comprise a 2-deoxy-2-amino-a-D-glucopyranose (glucosamine) in glycosidic linkage with an aminoalkyl (aglycon) group. Compounds are phosphorylated at the 4 or 6 carbon on the glucosamine ring and have three alkanoyloxyalkanoyl residues. The compounds are described generally by Formula I, wherein X represents an oxygen or sulfur atom, Y represents an oxygen atom or NH group, “n”, “m”, “p” and “q” are integers from 0 to 6, R1, R2, and R3 represent normal fatty acyl residues having 7 to 16 carbon atoms, R4 and R5 are hydrogen or methyl, R6 and R7 are hydrogen, hydroxy, alkoxy, phosphono, phosphonooxy, sulfo, sulfooxy, amino, mercapto, cyano, nitro, formyl or carboxy and esters and amides thereof; R8 and R9 are phosphono or hydrogen. The configuration of the 3′ stereogenic centers to which the normal fatty acyl residues are attached is R or S, but preferably R. The stereochemistry of the carbon atoms to which R4 or R5 are attached can be R or S. All stereoisomers, both enantiomers and diastereomers, and mixtures thereof, are considered to fall within the scope of the subject invention.

The heteroatom X of such compounds of the subject invention can be oxygen or sulfur. In a preferred embodiment, X is oxygen. Although the stability of the molecules could be affected by a substitution at X, the immunomodulating activity of molecules with these substitutions is not expected to change.

The number of carbon atoms between heteroatom X and the aglycon nitrogen atom is determined by variables “n” and “m”. Variables “n” and “m” can be integers from 0 to 6. In a preferred embodiment, the total number of carbon atoms between heteroatom X and the aglycon nitrogen atom is from about 2 to about 6 and most preferably from about 2 to about 4.

Such compounds are aminoalkyl glucosamine compounds which are phosphorylated. Compounds can be phosphorylated at position 4 or 6 (R8 or R9) on the glucosamine ring and are most effective if phosphorylated on at least one of these positions. In a preferred embodiment, R8 is phosphono and R9 is hydrogen.

Such compounds are hexaacylated, that is they contain a total of six fatty acid residues. The aminoalkyl glucosamine moiety is acylated at the 2-amino and 3-hydroxyl groups of the glucosamine unit and at the amino group of the aglycon unit with 3-hydroxyalkanoyl residues. In Formula I, these three positions are acylated with 3-hydroxytetradecanoyl moieties. The 3-hydroxytetradecanoyl residues are, in turn, substituted with normal fatty acids (R1-R3), providing three 3-n-alkanoyloxytetradecanoyl residues or six fatty acid groups in total.

The chain length of normal fatty acids R1-R3 can be from about 7 to about 16 carbons. Preferably, R1-R3 are from about 9 to about 14 carbons. The chain lengths of these normal fatty acids can be the same or different. Although, only normal fatty acids are described, it is expected that unsaturated fatty acids (i.e. fatty acid moieties having double or triple bonds) substituted at R1,-R3 on the compounds would produce biologically active molecules. Further, slight modifications in the chain length of the 3-hydroxyalkanoyl residues are not expected to dramatically effect biological activity.

Specific examples of AGP's include: CRX-527 which is disclosed in Stover et al., JBC 2004 279, No 6, page 4440-4449 (available at www.jbc.org/content/279/6/4440.full.pdf).

WO0212258 and WO3065806 disclose additional embodiments of AGPs having a cyclic aminoalkyl (aglycon) linked to a 2-deoxy-2-amino-a-D-glucopyranose (glucosaminide), commonly referred to as “cyclic AGP's.”

Reference generally to AGPs herein includes both cyclic and non cyclic AGPs.

Cyclic AGPs possess three 3-alkanoyloxyalkanoyl residues comprising a primary and secondary fatty acyl chain, each carbon chain consisting of from 2-24 carbon atoms, and preferably from 7-16 carbon atoms. In one preferred aspect each primary chain contains 14 carbon atoms and each secondary carbon chain has between 10 and 14 carbon atoms per chain.

The cyclic AGPs are described by the general formula II:

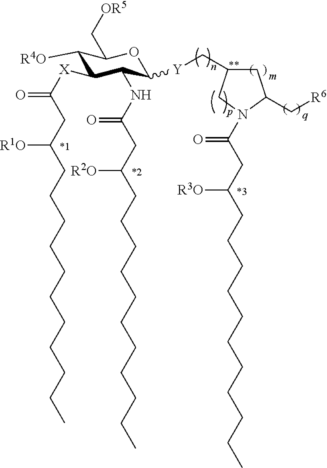

These compounds comprise a 2-deoxy-2-amino-p-D-glucopyranose(glucosamine)glycosidically linked to an cyclic aminoalkyl (aglycon) group. The compounds are phosphorylated at the 4 or 6-position of the glucosamine ring and acylated with alkanoyloxytetradecanoyl residues on the aglycon nitrogen and the 2 and 3-positions of the glucosamine ring. The compounds are described generally by formula (II): and pharmaceutically acceptable salts thereof, wherein X is —O— or NH— and Y is —O— or —S—; R1, R2, and R3 are each independently a (C2-C24) acyl group, including saturated, unsaturated and branched acyl groups; R4 is —H or —PO₃R7R8, wherein R7 and R8 are each independently H or (C1-C4)alkyl; R5 is —H, —CH₃ or —PO₃R9R10, wherein R9 and RI0 are each independently selected from —H and (CI-C4)alkyl; R6 is independently selected from H, OH, (CI-C4)alkoxy, —PO₃R11R12, —OPO₃R11R12, —SO₃R11, —OSO₃R11, —NR11R12, —SR11, —CN, —NO₂, —CHO, —CO₂R11, and —CONR11R12, wherein R11 and R12 are each independently selected from H and (CI-C4)alkyl; with the proviso that when R4 is -Po₃R7R8, R5 is other than —P0 R9R10, wherein“*1-3” and “**” represent chiral centers; wherein the subscripts n, m, p and q are each independently an integer from 0 to 6, with the proviso that the sum of p and m is from 0 to 6.

In some embodiments, the compounds of the present invention contain an —O— at X and Y, R4 is PO₃R7R8, R5 and R6 are H, and the subscripts n, m, p, and q are integers from 0 to 3. In a more preferred embodiment, R7 and R8 are —H. In an even more preferred embodiment, subscript n is 1, subscript m is 2, and subscripts p and q are 0. In yet an even more preferred embodiment, R1, R2, and R3 are tetradecanoyl residues. In a still more preferred embodiment, *1-3 are in the R configuration, Y is in the equatorial position, and ** is in the S configuration (N—[(R)-3-tetradecanoyloxytetradecanoyl]-(S)-2-pyrrolidinomethyl 2-deoxy-4-0-phosphono-2-[(R)-3-tetradecanoyloxytetradecanoylamino]-3-0-[(R)-3-tetradecanoyloxytetradecanoyl]-p-D-glucopyranoside and pharmaceutically acceptable salts thereof

Preferred cyclic structures include:

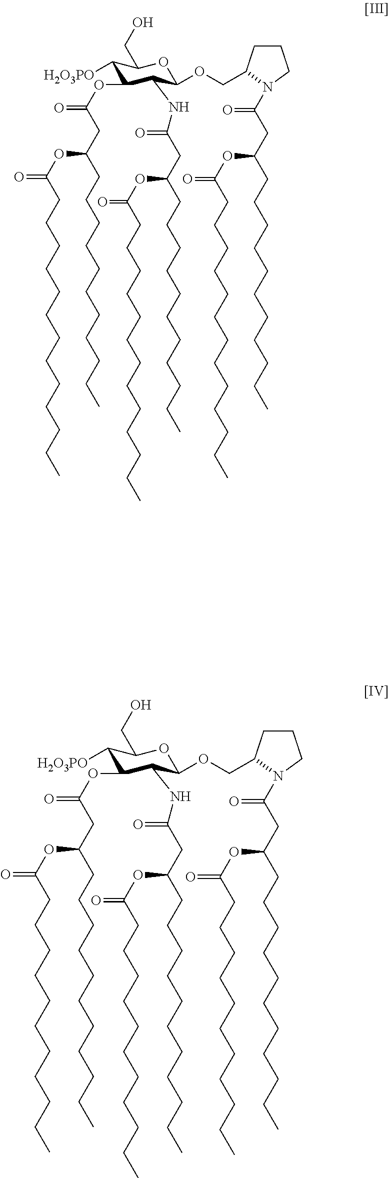

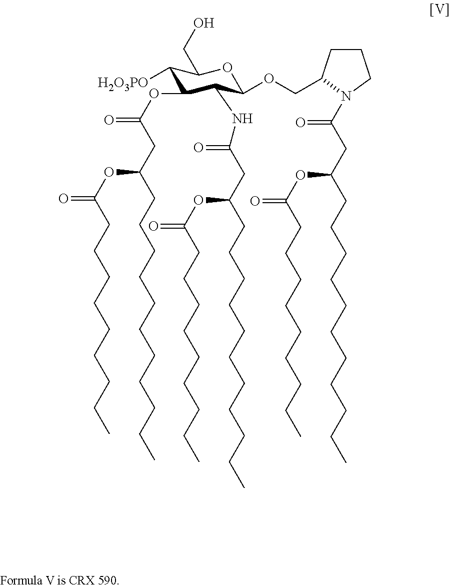

In another aspect the TLR4 receptor ligand is an AGP having one or more ether linked rather than ester linked primary and/or secondary lipid groups. In this embodiment, R1-R3 represent straight chain alkyl groups and not acyl groups, making the groups R1O—, R2O—, and R3O— alkoxy rather than alkanoyloxy groups and the attachment to the primary acyl chain an ether rather than an ester linkage. In the case of an ether-linked primary lipid group, the 3-alkanoyloxyalkanoyl residue attached to the 3-hydroxy group of the glucosamine unit is replaced with either a 3-alkanoyloxyalkyl moiety or a 3-alkoxyalkyl moiety, making the attachment of the primary lipid group to the glucosamine 3-position an ether rather than an ester linkage.

A general formula for ethers is that of formula IV of WO2006 016997.

An example of a preferred compound is CRX601.

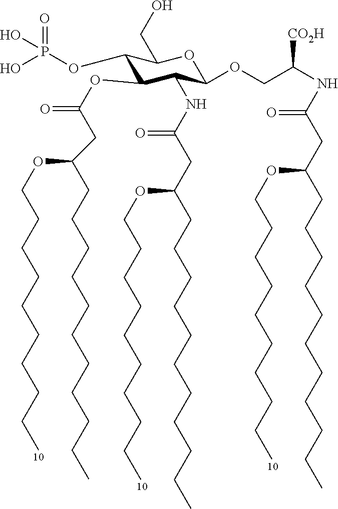

In another aspect, the AGP molecule may have different number of carbons in the molecule's primary chains and/or secondary chains. Such compounds are disclosed in WO04062599 and WO06016997. As with other AGPs, each carbon chain may consist of from 2-24 carbon atoms, and preferably from 7-16 carbon atoms. In one preferred aspect each primary chain contains 14 carbon atoms and each secondary carbon chain has between 10 and 14 carbon atoms per chain.

Such compounds are represented by the following structures:

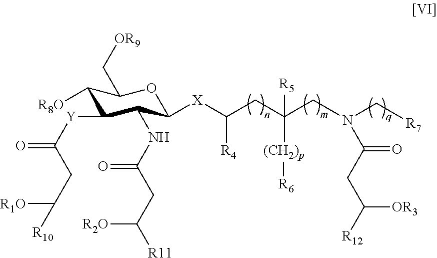

wherein X is selected from the group consisting of O and S at the axial or equatorial position; Y is selected from the group consisting of O and NH; n, m, p and q are integers from 0 to 6; R1, R2 and R3 are the same or different and are fatty acyl residues having from 1 to about 20 carbon atoms and where one of R1, R2 or R3 is optionally hydrogen; R4 and R5 are the same or different and are selected from the group consisting of H and methyl; R6 and R7 are the same or different and are selected from the group consisting of H, hydroxy, alkoxy, phosphono, phosphonooxy, sulfo, sulfooxy, amino, mercapto, cyano, nitro, formyl and carboxy, and esters and amides thereof; R8 and R9 are the same or different and are selected from the group consisting of phosphono and H, and at least one of R8 and R9 is phosphono; R10, R11 and R12 are independently selected from straight chain unsubstituted saturated aliphatic groups having from 1 to 10 carbon atoms; or a pharmaceutically acceptable salt thereof.

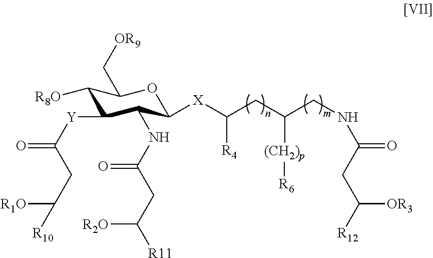

wherein X is selected from the group consisting of O and S at the axial or equatorial position; Y is selected from the group consisting of O and NH; n and m are 0; R1, R2 and R3 are the same or different and are fatty acyl residues having from 1 to about 20 carbon atoms and where one of R1, R2 or R3 is optionally hydrogen; R4 is selected from the group consisting of H and methyl; p is 1 and R6 is COOH or p is 2 and R6 is OP0₃H₂; R8 and R9 are the same or different and are selected from the group consisting of phosphono and H, and at least one of R8 and R9 is phosphono; and R10, R11 and R12 are independently selected from straight chain unsubstituted saturated aliphatic groups having from 1 to 10 carbon atoms; or a pharmaceutically acceptable salt thereof.

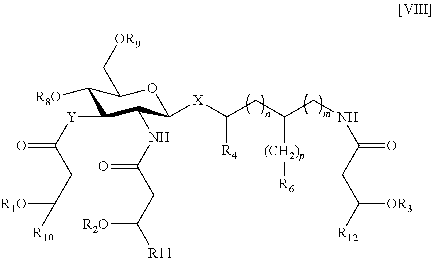

wherein X is selected from the group consisting of O and S at the axial or equatorial position; Y is selected from the group consisting of O and NH; n, m, p and q are integers from 0 to 6; R1, R2 and R3 are the same or different and are straight chain saturated aliphatic groups (i.e., straight chain alkyl groups) having from 1 to about 20 carbon atoms and where one of R1, R2 or R3 is optionally hydrogen; R4 and R5 are the same or different and are selected from the group consisting of H and methyl; R6 and R7 are the same or different and are selected from the group consisting of H, hydroxy, alkoxy, phosphono, phosphonooxy, sulfo, sulfooxy, amino, mercapto, cyano, nitro, formyl and carboxy, and esters and amides thereof; R8 and R9 are the same or different and are selected from the group consisting of phosphono and H, and at least one of R8 and R9 is phosphono; R10, R11 and R12 are independently selected from straight chain unsubstituted saturated aliphatic groups having from 1 to 11 carbon atoms;
or a pharmaceutically acceptable salt thereof.

The general formula may also comprise an R5 group, at the same position as shown in formula VI above, wherein R5 is selected from the group consisting of H and methyl.

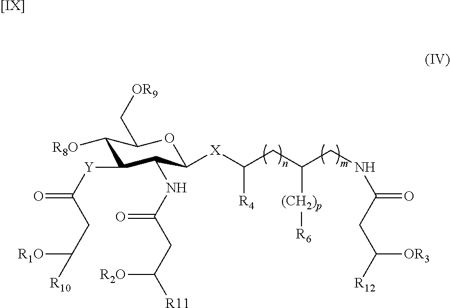

Yet another type of compound of this invention has the formula (IV): wherein Y is now fixed as oxygen; X is selected from the group consisting of O and S at the axial or equatorial position; n and m are 0; R1, R2 and R3 are the same or different and are fatty acyl residues having from 1 to about 20 carbon atoms and where one of R1, R2 or R3 is optionally hydrogen; R4 is selected from the group consisting of H and methyl; p is 0 or 1 and R6 is COOH, or p is 1 or 2 and R6 is OPO3H2; R8 and R9 are the same or different and are selected from the group consisting of phosphono and H, and at least one of R8 and R9 is phosphono; and R10, R11 and R12 are independently selected from straight chain unsubstituted saturated aliphatic groups having from 1 to 10 carbon atoms; or a pharmaceutically acceptable salt thereof.

These compounds thus have two acylated chains and one non-acylated ether chain.

Processes for making AGPs are also disclosed in WO0612425.

Other AGP structures such as CRX 524 are disclosed in INFECTION AND IMMUNITY, May 2005, p. 3044-3052 Vol. 73, No. 5.

In one embodiment the TLR agonist for use in combination with the antibody, or in combination with the antigen used to generate an antibody, is combined with another adjuvant, wherein the adjuvant may be an oil in water emulsion, such as AS03 from GSK or MF59, or a saponin such as QS21, or an aluminum salt or glucopyranosyl lipid A adjuvants (Immune Design)

A combination of TLR agonists may be used.

In one embodiment of the invention the TLR agonist described herein is delivered in the absence of an immunogen, such as in the absence of a polypeptide or polysaccharide antigen.

In one embodiment the TLR agonist described herein is provided in combination with a pharmaceutically acceptable excipient or additive.

In one aspect the TLR agonist is able to induce the production of CD11b+ phagocytic cells in the periphery.

In one aspect the TLR agonist is able to increase the phagocytic activity of CD11b+ phagocytic cells, suitably as measured in blood or sera from the periphery of an animal.

Phagocytotic activity may be determined by the assay described herein, in which the uptake of a labelled antigen, such as amyloid beta fragment, is detected. One suitable assay for phagocytosis of beta, which forms an aspect of this invention, comprises incubation of lineage CD11b+ monocytes with a labeled amyloid beta polypeptide and detection of the uptake of the labeled amyloid beta into the cells.

In one aspect, the invention relates to a method for monitoring of vaccine efficacy of an Alzheimer's vaccine, the method comprising contacting phagocytic cells with a labeled amyloid protein, or fragment thereof, wherein the cells are obtained from the blood of an individual vaccinated with a vaccine, followed by detection of the uptake of the labeled amyloid beta into the cells.

TLR agonists may be combined with other components which are effective to stimulate the innate immune system.

The compositions and methods of the present invention may be used in one or more of preventing or reducing effect on deposits of amyloid protein, stimulation of innate immunity via microglia cells, increasing amyloid phagocytosis and preventing or reducing behaviours that are associated with disease states such as Alzheimer's disease. The examples provided herein give suitable methods for assessing of these parameters.

Beta amyloid deposits may be measured as a function of the area of plaques in a brain section, or assessed by total protein concentration, as described in the attached Examples. Other suitable methods are disclosed in WO2009105641.

Effects of the treatments and compositions of the invention on behaviour associated with Alzheimer's disease may be assessed in human patients, or in animal models, for example. Suitable animal models include the mouse APP model for Alzheimer's disease, the PS1 mouse model and the APP/PS1 model. See Richard, K. L. et al. J Neurosci 28, 5784-5793 (2008).

Suitable animal (rodent) tests include one or more of the T-water maze test, Passive avoidance test, or nesting behaviour tests as described herein (Filali M, et al Cognitive and non-cognitive behaviours in an APPswe/PS1 bigenic model of Alzheimer's disease. Genes Brain Behav. 2009 March; 8(2):143-8. Epub 2008 Dec. 3. PubMed PMID: 19077180.). Other behavioural tests that may be employed are described in WO2009105641, incorporated herein by reference.

Stimulation of the innate immune system may be effected by, and/or measured by, stimulation of microglia. In another aspect the innate immune response may be assessed by the triggering transcriptional activation of TLR2 in brain tissues, for example in appropriate animal mouse models.

The methods and compositions of the present invention may be used to protect or treat a mammal by means of administering via the systemic or mucosal route. These administrations may include injection via the intramuscular (i.m.), intraperitoneal (i.p.), intradermal or subcutaneous routes; or via mucosal administration to the oral/alimentary, sublingual, intranasal, respiratory, genitourinary tracts. The administration may include an ocular administration or administration by antigen loaded patches. The composition of the invention may be administered as a single dose, or multiple doses. In addition, the compositions of the invention may be administered by different routes for priming and boosting, for example, IM priming doses and IN for booster doses.

In one aspect intramuscular delivery of low doses of 3D MPL is not preferred. In one aspect the amount of 3D MPL used is equivalent to 50 ug 3D MPL injected intraperitonealy

In one aspect 3D MPL is delivered by intraperitoneal injection.

The components of the present invention, such as the TLR agonist, antibody or antigen, may be administered alone or with suitable pharmaceutical carriers, and can be in solid or liquid form, such as tablets, capsules, powders, solutions, suspensions, or emulsions.

TLR agonists such as aminoalkyl glucosaminide phosphates (AGP), 3D MPL or MPL or any component of the invention, may be formulated into a “vaccine,” and administered in free solution, or formulated with an adjuvant, or excipient. Vaccine preparation is generally described in Vaccine Design (“The subunit and adjuvant approach” (eds Powell M. F. & Newman M. J.) (1995) Plenum Press New York). Encapsulation within liposomes is described by Fullerton, U.S. Pat. No. 4,235,877. The vaccines of the present invention may be stored in solution or lyophilized.

Effective doses of the components of the present invention, e.g. for the treatment of a subject having amyloid deposits or AD, vary depending upon many different factors, including means of administration, target site, physiological state of the patient, other medications administered, physical state of the patient relative to other medical complications, and whether treatment is prophylactic or therapeutic. Treatment dosages need to be titrated to optimize safety and efficacy. The amount of TLR agonist may depend on whether other components, such as an adjuvant are also administered. Subject doses of the TLR agonist described herein typically range from about 0.1 μg to 50 mg per administration, which depending on the application could be given daily, weekly, or monthly and any other amount of time there between. More typically mucosal or local doses range from about 10 μg to 10 mg per administration, and optionally from about 100 μg to 1 mg, with 2-4 administrations being spaced days or weeks apart. More typically, immune stimulant doses range from 1 μg to 10 mg per administration, and most typically 10 μg to 1 mg, with daily or weekly administrations. Doses of the compounds described herein for parenteral delivery e.g., for inducing an innate immune response, or in specialized delivery vehicles typically range from about 0.1 μg to 10 mg per administration, which depending on the application could be given daily, weekly, or monthly and any other amount of time therebetween. More typically parenteral doses for these purposes range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above. Suitable doses of antibody and antigen needed to demonstrate an effect in vivo, or to elicit an immune response in vivo respectively, are well known to the skilled person.

Compositions of the invention may comprise a TLR4 agonist in an amount of between about 1 μg to about 100 μg, for example between about 1 μg and about 60 μg or between 10 μg and about 50 μg, for example, about 10 μg, about 12.5 μg, about 15 μg, about 20 μg, about 25 μg, about 30 μg, about 40 μg or in particular about 50 μg. In particular, QS21 is present in an amount between about 40 μg and 60 μg or between 45 and 55 μg or between 47 and 53 μg or between 48 and 52 μg or between 49 and 51 or about 50 μg. Alternatively QS21 is present in an amount between 21 μg and 29 μg or between about 22 μg and about 28 μg or between about 23 μg and about 27 μg or between about 24 μg and about 26 μg, or about 25 μg.

In a further embodiment compositions of the invention comprise a TLR4 agonist in an amount of about 10 μg, for example between about 5 μg and 15 μg, about 6 μg and about 14 μg, about 7 μg and about 13 μg, about 8 μg and about 12 μg or about 9 μg and about 11 μg, or about 10 μg.

In a further embodiment, compositions of the invention comprise a TLR4 agonist in an amount of around about 5 μg.

A suitable amount of TLR4 agonist in the compositions of the invention is for example any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 μg.

Compositions of the invention may comprise and adjuvant containing MPL, QS21 and liposome (50 μg MPL and 50 μg QS21 human dose).

The present invention further shows that the use of a detoxified form of QS21 by the addition of liposome is able to increase the phagocytosis of amyloid, and therefore reduce the amount of Aβ present within the blood and hence within the central nervous system. A similar increase in the phagocytic activity of the resident microglial cells or freshly recruited resulting phagocytic cells in the brain may improve the clearance of the pathogenic Aβ.

Thus in one aspect the invention relates to a method of preventing and/or reducing amyloid deposition in a subject comprising stimulating the innate immune system of an individual using the methods and compositions of the invention, including but not limited to those comprising detoxified QS21, under conditions effective to prevent or reduce amyloid deposits. This prevention or reduction may be achieved by enhancing uptake, and optionally intracellular degradation, of the amyloid in cells of the immune system.

The stimulated cells may be monocytes, the circulating precursors of macrophages, microglial cells or their precursors and dendritic cells, or any phagocytic cell as disclosed herein. Reference to phagocytosis and stimulation of phagocytosis herein, may be read more generally to include uptake of a material (e.g. an antigen) into a cell of the immune system. Likewise reference to cells capable of phagocytosis include reference to immune cells capable of antigen uptake, for example uptake of antigen bound to an antibody. As examples of uptake mechanisms, phagocytosis and macropinocytosis are specific examples, and the invention specifically contemplates both.

In a particular aspect the invention relates to use of compositions as disclosed herein to increase uptake of amyloid by monocytes in the peripheral blood. The mode of action of the immunotherapy may rely both on an increased number of monocytes and their degree of activation, as determined by the presence of CD11b and/or Ly6C markers.

### QS21 and Liposomes

Compositions of the invention comprise the saponin QS21 (WO8809336A1; U.S. Pat. No. 5,057,540A). QS21 is well known in the art as a natural saponin derived from the bark of Quillaja saponaria Molina, which induces CD8+ cytotoxic T cells (CTLs), Th1 cells and a predominant IgG2a antibody response. For the avoidance of doubt reference to QS21 includes OPT-821 or PQS-21 (Ragupathi et al., 2010) Ref: (Ragupathi G, Damani P, Deng K, Adams M M, Hang J, George C, Livingston P O, Gin D Y. Preclinical evaluation of the synthetic adjuvant SQS-21 and its constituent isomeric saponins. Vaccine. 2010 Jun. 11; 28(26):4260-7. PubMedPMID: 20450868; PubMed Central PMCID: PMC2882175.

In a suitable form of the present invention, the compositions of the invention comprise QS21 in substantially pure form, that is to say, the QS21 is at least 80%, at least 85%, at least 90% pure, for example at least 95% pure, or at least 98% pure.

Compositions of the invention comprise QS21 in an amount of between about 1 μg to about 100 μg, for example between about 1 μg and about 60 μg or between 10 μg and about 50 μg, for example, about 10 μg, about 12.5 μg, about 15 μg, about 20 μg, about 25 μg, about 30 μg, about 40 μg or in particular about 50 μg. In particular, QS21 is present in an amount between about 40 μg and 60 μg or between 45 and 55 μg or between 47 and 53 μg or between 48 and 52 μg or between 49 and 51 or about 50 μg. Alternatively QS21 is present in an amount between 21 μg and 29 μg or between about 22 μg and about 28 μg or between about 23 μg and about 27 μg or between about 24 μg and about 26 μg, or about 25 μg.

In a further embodiment compositions of the invention comprise QS21 in an amount of about 10 μg, for example between about 5 μg and 15 μg, about 6 μg and about 14 μg, about 7 μg and about 13 μg, about 8 μg and about 12 μg or about 9 μg and about 11 μg, or about 10 μg.

In a further embodiment, compositions of the invention comprise QS21 in an amount of around about 5 μg, for example between about 1 μg and 9 μg, about 2 μg and about 8 μg, about 3 μg and about 7 μg, about 4 μg and about 6 μg, or about 5 μg.

A suitable amount of QS21 in the compositions of the invention is for example any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 μg.

Compositions of the invention comprising QS21 and a sterol, cholesterol in particular, show a decreased reactogenicity when compared to compositions in which the sterol is absent, while the adjuvant effect is maintained. Reactogenicity studies may be assessed according to the methods disclosed in WO 96/33739. Suitably the sterol is associated to the saponin adjuvant as described in WO 96/33739. In a particular embodiment, the cholesterol is present in excess to that of QS21, for example, the ratio of QS21:sterol will typically be in the order of 1:100 to 1:1 (w/w), suitably between 1:10 to 1:1 (w/w), and preferably 1:5 to 1:1 (w/w). In particular, the ratio of QS21:sterol being at least 1:2 (w/w). In a particular embodiment, the ratio of QS21:sterol is 1:5 (w/w). Suitable sterols include β-sitosterol, stigmasterol, ergosterol, ergocalciferol and cholesterol. In one particular embodiment, the compositions of the invention comprise cholesterol as sterol. These sterols are well known in the art, for example cholesterol is disclosed in the Merck Index, 11th Edn., page 341, as a naturally occurring sterol found in animal fat.

The compositions of the invention comprise QS21 in its less reactogenic composition where it is quenched with an exogenous sterol, such as cholesterol for example. As used herein, the term “detoxified QS21” refers to QS21 in combination with a sterol. Several particular forms of less reactogenic compositions wherein QS21 is quenched with an exogenous cholesterol exist. In a specific embodiment, the saponin/sterol is in the form of a liposome structure (WO 96/337391).

The term “liposome(s)” generally refers to uni- or multilamellar (particularly 2, 3, 4, 5, 6, 7, 8, 9, or 10 lamellar depending on the number of lipid membranes formed) lipid structures enclosing an aqueous interior. Liposomes and liposome formulations are well known in the art. Lipids which are capable of forming liposomes include all substances having fatty or fat-like properties. Lipids which can make up the lipids in the liposomes may be selected from the group comprising glycerides, glycerophospholipides, glycerophosphinolipids, glycerophosphonolipids, sulfolipids, sphingolipids, phospholipids, isoprenolides, steroids, stearines, sterols, archeolipids, synthetic cationic lipids and carbohydrate containing lipids.

In a particular embodiment the liposomes of the invention comprise a phospholipid. Suitable phospholipids include (but are not limited to): phosphocholine (PC) which is an intermediate in the synthesis of phosphatidylcholine; natural phospholipid derivates: egg phosphocholine, egg phosphocholine, soy phosphocholine, hydrogenated soy phosphocholine, sphingomyelin as natural phospholipids; and synthetic phospholipid derivates: phosphocholine (didecanoyl-L-α-phosphatidylcholine [DDPC], dilauroylphosphatidylcholine [DLPC], dimyristoylphosphatidylcholine [DMPC], dipalmitoyl phosphatidylcholine [DPPC], Distearoyl phosphatidylcholine [DSPC], Dioleoyl phosphatidylcholine [DOPC], 1-palmitoyl, 2-oleoylphosphatidylcholine [POPC], Dielaidoyl phosphatidylcholine [DEPC]), phosphoglycerol (1,2-Dimyristoyl-sn-glycero-3-phosphoglycerol [DMPG], 1,2-dipalmitoyl-sn-glycero-3-phosphoglycerol [DPPG], 1,2-distearoyl-sn-glycero-3-phosphoglycerol [DSPG], 1-palmitoyl-2-oleoyl-sn-glycero-3-phosphoglycerol [POPG]), phosphatidic acid (1,2-dimyristoyl-sn-glycero-3-phosphatidic acid [DMPA], dipalmitoyl phosphatidic acid [DPPA], distearoyl-phosphatidic acid [DSPA]), phosphoethanolamine(1,2-dimyristoyl-sn-glycero-3-phosphoethanolamine [DMPE], 1,2-Dipalmitoyl-sn-glycero-3-phosphoethanolamine [DPPE], 1,2-distearoyl-sn-glycero-3-phosphoethanolamine DSPE 1,2-Dioleoyl-sn-Glycero-3-Phosphoethanolamine [DOPE]), phosphoserine, polyethylene glycol [PEG] phospholipid (mPEG-phospholipid, polyglycerin-phospholipid, funcitionilized-phospholipid, terminal activated-phospholipid). In one embodiment the liposomes comprise 1-palmitoyl-2-oleoyl-glycero-3-phosphoethanolamine. In one embodiment highly purified phosphatidylcholine is used and can be selected from the group comprising Phosphatidylcholine (EGG), Phosphatidylcholine Hydrogenated (EGG), Phosphatidylcholine (SOY) and Phosphatidylcholine Hydrogenated (SOY). In a further embodiment the liposomes comprise phosphatidylethanolamine [POPE] or a derivative thereof.

Liposome size may vary from 30 nm to several μm depending on the phospholipid composition and the method used for their preparation. In particular embodiments of the invention, the liposome size will be in the range of 50 nm to 500 nm and in further embodiments 50 nm to 200 nm. Dynamic laser light scattering is a method used to measure the size of liposomes well known to those skilled in the art.

In particular liposomes of the invention may comprise dioleoyl phosphatidylcholine [DOPC] and a sterol, in particular cholesterol. Thus, in a particular embodiment, compositions of the invention comprise QS21 in any amount described herein in the form of a liposome, wherein said liposome comprises dioleoyl phosphatidylcholine [DOPC] and a sterol, in particular cholesterol.

Compositions of the invention may or may not comprise one or more further immunostimulants. In a particular embodiment, the compositions of the invention as described herein do not comprise a lipopolysaccharide, particularly a non-toxic derivative of lipid A, particularly monophosphoryl lipid A or more particularly 3-Deacylated monophosphoryl lipid A (3D—MPL). 3D-MPL is sold under the name MPL by GlaxoSmithKline Biologicals N.A. and is referred throughout the document as MPL or 3D-MPL. see, for example, U.S. Pat. Nos. 4,436,727; 4,877,611; 4,866,034 and 4,912,094. 3D-MPL primarily promotes CD4+T cell responses with an IFN-g (Th1) phenotype. 3D-MPL can be produced according to the methods disclosed in GB 2 220 211 A. Chemically it is a mixture of 3-deacylated monophosphoryl lipid A with 3, 4, 5 or 6 acylated chains. Compositions comprising increased amount of hexa-acyl conger (3-deacylated monophosphoryl lipid A with 6 acylated chains are preferred) and can be obtained by the methods described in WO02/078637 and US20070212758.

In a further particular embodiment, the compositions of the invention as described herein do not comprise an immunostimulatory oligonucleotide, particularly an immunostimulatory oligonucleotide comprising one or more CpG motifs.

In a further particular embodiment, the compositions of the invention as described herein do not comprise an immunostimulatory oligonucleotide, particularly an immunostimulatory oligonucleotide comprising one or more CpG motifs or a lipopolysaccharide, particularly a non-toxic derivative of lipid A, particularly monophosphoryl lipid A or more particularly 3-Deacylated monophosphoryl lipid A (3D—MPL).

Any aspect or feature of the invention may be combinable with any other aspect or feature of the invention, even where disclosed in a specific example, except where obvious from the context. For example, antigens, adjuvants or TLR agonists disclosed in any aspect or feature are combinable with any other aspect or feature of the invention.

For the avoidance of doubt the terms ‘comprising’, ‘comprise’ and ‘comprises’ herein is intended by the inventors to be optionally substitutable with the terms ‘consisting of’, ‘consist of’, and ‘consists of’, respectively, in every instance. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “about” (or “around”) in all numerical values allows for a 5% variation, i.e. a value of about 1.25% would mean from between 1.19%-1.31%.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the measurement, the method being employed to determine the value, or the variation that exists among the study subjects.

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, BBC, AAABCCCC, CBBAAA, CABABB, and so forth. The skilled artisan will understand that typically there is no limit on the number of items or terms in any combination, unless otherwise apparent from the context.

While the compositions and methods of this disclosure have been described in terms of suitable embodiments, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

It will be understood that particular embodiments described herein are shown by way of illustration and not as limitations of the disclosure. The principal features of this disclosure can be employed in various embodiments without departing from the scope of the disclosure. Those skilled in the art will recognize, or be able to ascertain using no more than routine study, numerous equivalents to the specific procedures described herein. Such equivalents are considered to be within the scope of this disclosure and are covered by the claims.

Certain features of the present invention include the following:

A A method of preventing and/or reducing amyloid deposition in a subject comprising treatment of a subject with an effective amount of a composition consisting or consisting essentially of QS21 formulated in a liposome comprising a sterol.
B A composition consisting or consisting essentially of QS21 formulated in a liposome comprising a sterol for use in preventing and/or reducing amyloid deposition in a subject.
C Use of a composition consisting or consisting essentially of QS21 formulated in a liposome comprising a sterol in the manufacture of a medicament for preventing and/or reducing amyloid deposition in a subject.
D A method of preventing and/or treating Alzheimer's disease, macular degeneration, Parkinson's disease, islet amyloid deposits in pancreas, ALS or Huntington's disease in a subject comprising treatment of a subject with an effective amount of a composition consisting or consisting essentially of QS21 formulated in a liposome comprising a sterol.
E A composition consisting or consisting essentially of QS21 formulated in a liposome comprising a sterol for use in preventing and/or reducing treating Alzheimer's disease, macular degeneration, Parkinson's disease or Huntington's disease.
F Use of a composition consisting or consisting essentially of QS21 formulated in a liposome comprising a sterol for preventing and/or treating Alzheimer's disease, macular degeneration, Parkinson's disease, islet amyloid deposits in pancreas, ALS or Huntington's disease.
G A composition comprising a β-amyloid antigen and QS21 formulated in a liposome comprising a sterol.
H A composition according to G, wherein the β-amyloid antigen comprises or consists of Aβ1-5, Aβ1-6, Aβ1-7, Aβ1-10, Aβ1-14, Aβ1-15, Aβ2-7, Aβ2-8, Aβ3-7, Aβ3-8, Aβ11-16, Aβ11-17, Aβp(E)3-7, Aβp(E)3-8, Aβp(E)3-40, Aβp(E)3-42, Aβp(E)11-16, Aβp(E)11-17, Aβp(E)11-40 or Aβp(E)11-42.
I A composition according to G or H, which further comprises the β-amyloid antigen Aβ1-6.
J A composition according to G or H, which further comprises the β-amyloid antigen Aβp(E)3-8.
K A composition according to G or H, which further comprises the β-amyloid antigen Aβp(E)11-16.
L A composition according to any one of G to K for use in medicine.
M A composition according to any one of G to K for use in preventing and/or treating Alzheimer's disease in a subject.
N Use of a composition according to any one of G to K in the manufacture of a medicament for preventing and/or treating Alzheimer's disease in a subject.
O A method of preventing and/or treating Alzheimer's disease in a subject comprising treatment of a subject with an effective amount of a composition according to any one of claims G to K.
P A composition, method or use according to any of A to O wherein the sterol is cholesterol.
Q A composition, method or use according to any of A to P wherein the liposome comprises DOPC.
R A composition, method or use according to any of A to Q wherein the ratio of QS21 to cholesterol is between 1:1 and 1:100, for example between 1:2 and 1:5.
S A composition, method or use according to any of A to R wherein the QS21 is present in an amount between about 1 and about 100 μg, for example between 10 μg and 60 μg, for example between 40 μg and 60 μg, for example about 50 μg.
T A composition, method or use according to any of A to S which does not comprise an immunostimulatory oligonucleotide, particularly an immunostimulatory oligonucleotide comprising one or more CpG motifs, a lipopolysaccharide, particularly a non-toxic derivative of lipid A, particularly monophosphoryl lipid A or more particularly 3-Deacylated monophosphoryl lipid A (3D—MPL), or a combination thereof.
U A kit comprising: i) a composition consisting or consisting essentially of QS21 formulated in a liposome comprising a sterol.
V A kit according to U further comprising: ii) a β-amyloid antigen.
W A kit according to either U or V wherein the QS21 is present in an amount between about 1 and about 100 μg, for example between 10 μg to 60 μg, for example between about 40 μg and 60 μg, for example about 50 μg.
X A kit according to any of U to W wherein the sterol is cholesterol.
Y A kit according to any of U to X wherein the liposome comprises DOPC.
Z A kit according to any one of U to Y wherein the ratio of QS21 to cholesterol is between 1:1 and 1:100, for example between 1:2 and 1:5.

The disclosure will be further described by reference to the following, non-limiting, examples:

### EXAMPLES

### General: Methods and Materials

All experiments with animals and related assays were performed in accordance with the Canadian Council on Animal Care (CCAC) guidelines for animal experimentation. Eight week old female C57BL/6 mice were obtained from Charles-Rivers laboratories (St-Constant, Quebec). The APP-PS1 mouse model was obtained from Jackson laboratories, stock 5866 (Savonenko et al., 2005 Savonenko A; Xu G M; Melnikova T; Morton J L; Gonzales V; Wong M P; Price D L; Tang F; Markowska A L; Borchelt D R. 2005. Episodic-like memory deficits in the APPswe/PS1dE9 mouse model of Alzheimer's disease: relationships to beta-amyloid deposition and neurotransmitter abnormalities. Neurobiol Dis 18(3):602-17). Intramuscular injections in mice were performed on either the gastrocnemius anterior in 50 or 25 μL depending on the experiments. Intravenous injections (100 μL) were performed in the tail vein.

The adjuvant compositions used were as follows:

### Adjuvant Composition:

For AS01B, AS03 and AS15 the full mouse dose is equal to 1/10 of a human dose.

AS01B is an Adjuvant System containing 3DMPL, QS21 and liposome (50 μg 3 DMPL and 50 μg QS21 human dose). The mouse dose of AS01B contains 5 ug of 3DMPL and 5 ug of QS21. Those doses were injected using the intramuscular route (i.m.) 25 ul per mouse of AS01B+25 ul of PBS (phosphate buffer saline) or 25 ul of the appropriate peptide.

AS03 is an Adjuvant System containing α-Tocopherol and squalene in an o/w emulsion. AS03 used in this study contained 23.72 mg/mL α-tocopherol, 21.38 mg/mL squalene and 9.72 mg/mL polysorbate-80 in PBS. The mean particle sizes of the adjuvant emulsions were determined by dynamic light scattering (Zeta-Nanosizer, Malvern Instruments). The final injection volumes (with or without vaccine) were 50 μl. PBS was used as diluents. A human dose of an AS03_{A}-adjuvanted vaccine contains 11.86 mg α-tocopherol, 10.69 mg squalene and 4.86 mg polysorbate-80. Mice were therefore injected with an adjuvant dose no greater than 1/10th of an adult human dose.

CRX601/AS03: 2 μg of CRX601 was diluted in PBS for a final volume of 25 μl. The 25 μl of CRX601 was added slowly to the 25 μl AS03 (2×concentrated). The vaccine was mixed by magnetic stirring at medium speed. Formulation was performed extemporaneously, the injections occurred within 60 min following the end of the formulation.

AS15 is an Adjuvant System containing 3DMPL, QS21, CpG and liposome (50 μg 3DMPL, 50 μg QS21 and 420 μg CpG)

“3-O-desacyl-4′-monophosphoryl lipid A” 3D-MPL: is an immunostimulant derived from the lipopolysaccharide (LPS) of the Gram-negative bacterium Salmonella minnesota. MPL has been deacylated and is lacking a phosphate group on the lipid A moiety. This chemical treatment dramatically reduces toxicity while preserving the immunostimulant properties. In the Figures and examples, all references to MPL are references to 3D-MPL)

QS21: is a natural saponin molecule extracted from the bark of the South American tree Quillaja saponaria Molina. A purification technique developed to separate the individual saponins from the crude extracts of the bark, permitted the isolation of the particular saponin, QS21, which is a triterpene glycoside demonstrating stronger adjuvant activity and lower toxicity as compared with the parent component. QS21 has been shown to activate MHC class I restricted CTLs to several subunit antigens, as well as to stimulate antigen specific lymphocytic proliferation.

CpG ODN 7909 is a synthetic single-stranded phosphorothioate oligodeoxy-nucleotide (ODN) of 24 bases length. Its base sequence is 5′-T CG T CG TTTTG-T CG TTTTGT CG TT-3′ (SEQ ID NO:12).

Formulations were performed on the days of injections. The volume of injection for one mouse was 50 μl. A typical formulation contains as follows: 20 μg-25 μg antigen was diluted with H₂O and PBS pH 7.4 for isotonicity.

### Examples 1-2

### Quantification of Anti-Amyloid Beta 1-42 Antibodies in Mice Serum Using ELISA

Whole blood is collected from mice and centrifuged on a vacutainer blood collection tube containing gel for serum separation. Serum samples are stored at −80° C. Streptavidin-coated plates (Greiner Bio-One, Germany) are first coated with beta-amyloid (1-42)-Lys(Biotin)-NH2 peptide (Anaspec, Inc.) at 0.5 μg/mL, using 50 mM sodium carbonate buffer, overnight at 4° C. Plates are then washed using a 4 times using PBS/0.05% Tween 20. Super Block (ScyTek laboratories) is added to the plates and incubated at 37° C. for at least one hour. Serum samples and standard (anti Aβ42 antibody (6E10 antibody, Covance, Inc.) are serially diluted in the plates and incubated at 37° C. for 2 hours. After a wash step, diluted peroxidase AffiniPure goat anti-mouse IgG, Fcγ fragment specific (Jackson ImmunoResearch Laboratories Inc.) is added for 1 hour at 37° C. A last wash is performed before adding TMB substrate reagent (BD OptEIA™, BD Biosciences) for 30 min at RT. Immediately, plates are stopped using 2N sulfuric acid, and then read at 450 nm using SpectraMax microplate reader (Molecular Devices, Inc.).

### Examples 3, 9-13, 19

### Monocyte Analysis and Counting after Adjuvant Injection in Mice

24-Hours after injection of the TLR adjuvants, peripheral blood was drawn from C57BL/6 mice via cardiac puncture with lithium-heparin as anticoagulant. Red blood cell lysis was performed twice on pooled blood with Ammonium Chloride-based Buffer (Sigma, Steinheim, Germany) and cells were counted with the EasyCount™ System (Immunicon). After one washing step, 500,000 cells were incubated with Rat anti-Mouse CD16/CD32 (BD Fc Block™ by BD Biosciences) for 10 min. on ice and cells were further incubated for 30 min. with a combination of the following directly conjugated antibodies at their pre-determined optimal concentration as described by Mildner et al., 2007 (Mildner A et al. Nat Neurosci. 2007 December; 10(12): 1544-53): PerCP labeled-Streptavidin, PE-Hamster anti-Mouse CD3, Rat anti-Mouse CD45R/B220, Rat anti-Mouse Ly-6G, Mouse anti-Mouse NK1.1 APC-conjugated Rat anti-Mouse CD11 b, PE-Cy7-conjugated Hamster anti-Mouse CD11c, FITC-Rat Anti-Mouse Ly-6C (all from BD Biosciences) and Pacific Blue™ Rat anti-Mouse CD62L (BioLegend, San Diego, Calif.). Cells were finally washed three times and fixed for 15 min. with a 2% paraformaldehyde solution in PBS. Fluorescence minus one (FMO) controls were always included in the assays for fluorescent compensation setting. Samples were acquired on a flow cytometer (BD FACSCanto II) and data analyzed with the FACSDiva software (BD Biosciences). Monocytes were identified by their Side/Forward scatter properties and gated as CD3-/CD45R/B220-/Ly-6G-/NK1.1-(Lineage-)/CD11b+ cells. CD11b+ monocyte frequency was reported as a percentage of the total cells excluding debris.

### Examples 4-6, 8, 14, 20

### Ex Vivo Uptake Assay of Aβ1-42

Preparation of cells: Peripheral blood was drawn from C57BL/6 mice via cardiac puncture with lithium-heparin as anticoagulant, 24-hours after injection of the adjuvants used herein. Red blood cell lysis was performed twice on pooled blood with Ammonium Chloride-based Buffer (Sigma, Steinheim, Germany) and cells were counted with the EasyCount™ System (Immunicon).

Cell stimulation/Aβ phagocytosis: cells were seeded at 10⁶ cells/mL onto a 24-well tissue culture plate and stimulated for 2 h or 24 h in the presence or absence of 1 μg/ml of Aβ1-42 HiLyte Fluor™488 (Anaspec, Fremont, Calif.), which was pre-incubated or not for 1 h with 1 μg/ml of anti-amyloid β antibodies (e.g. anti-Aβ1-17 IgG1, clone 6E10, Signet Laboratories, Dedham, Mass.) or with purified IgG from mouse serum (Sigma), as control.

FACS analysis: cells were harvested after incubation with fluorescent Aβ peptide with Trypsin/EDTA and cold PBS and washed three times. 500,000 cells were incubated in 96-well plate for 10 min. on ice in the presence of Rat anti-Mouse CD16/CD32 (clone 2.4G2—BD Fc Block™) and further stained for 30 min. with a combination of the following directly conjugated antibodies at their pre-determined optimal concentration: PE-Hamster anti-Mouse CD3 (clone 145-2C11), Rat anti-Mouse CD45R/B220 (clone RA3-6B2), Rat anti-Mouse Ly-6G (clone 1A8), Mouse anti-Mouse NK1.1 (clone PK136), APC-conjugated Rat anti-Mouse CD11b (clone M1/70), PE-Cy7-conjugated Hamster anti-Mouse CD11c (clone HL3), (all from BD PharMingen). Cells were finally washed twice and fixed for 15 min. with a 2% paraformaldehyde solution in PBS. FMOs controls were always included in the assays.

Samples were acquired on a flow cytometer (BD FACSCanto II) and data analyzed with the FACSDiva software (BD Biosciences).

Monocytes were identified by their Side/Forward scatter properties, excluding debris and gated as CD3-/CD45R/B220-/Ly-6G-/NK1.1-(Lineage-)/CD11b+ cells. Aβ uptake was assessed by reporting the percentage and Mean Fluorescence Intensity (GeoMean) of positive HiLyte fluor488 Aβ1-42 cells among gated monocytes.

### Example 7

### In Vivo Uptake Assay of Aβ42 after Immunization or Passive Immunotherapy

Mice: C57BL/6 female mice were purchased from Charles River. Three immunization groups were: 1—PBS injected via the intra muscular route; 2—AS03/CRX601 (the full mouse dose of AS03= 1/10 human dose of AS03) co-formulated with 2 μg of CRX601 per mouse plus Aβ1-6 conjugated to CRM (3 ug peptide dose); 3: The passive transfer in the tail vein of 150 ug of the 2E7 anti Aβ antibody.

Mice were immunized three times (at Day 0, 14 and 21) intramuscularly with (AS03-CRX601+Aβ1-6CRM; 1 μg per mouse based on peptide content) or by the passive immunotherapy using anti Aβ42 monoclonal antibody (150 ug of 2E7 antibody per mouse). At day 22, 5 μg per mouse of fluorescent HiLyte Fluor™ 488-labeled Amyloid β1-42 (Anaspec, Fremont, Calif.) was injected in the tail vein. Preparation of cells: Peripheral blood was drawn from immunized C57BL/6 mice via cardiac puncture with lithium-heparin as anticoagulant, 2 hours after i.v. injection of the Aβ1-42 HiLyte Fluor™ 488 (Day 22). Plasma of pooled blood was decanted and saved for measurement of antibody titres by ELISA. Red blood cell lysis was performed twice with Ammonium Chloride-based Buffer (Sigma, Steinheim, Germany) and cells were counted with the EasyCount™ System (Immunicon).

FACS analysis: 500,000 cells were aliquoted in a 96-well plate, washed once, incubated for 10 min. on ice in the presence of Rat anti-Mouse CD16/CD32 (clone 2.4G2—BD Fc Block™) and further stained for 30 min. with a combination of the following directly conjugated antibodies at their pre-determined optimal concentration: PE-Hamster anti-Mouse CD3 (clone 145-2C11), Rat anti-Mouse CD45R/B220 (clone RA3-6B2), Rat anti-Mouse Ly-6G (clone 1A8), Mouse anti-Mouse NK1.1 (clone PK136), APC-conjugated Rat anti-Mouse CD11b (clone M1/70), PE-Cy7-conjugated Hamster anti-Mouse CD11c (clone HL3), (all from BD PharMingen). Cells were finally washed twice and fixed for 15 min. with a 2% paraformaldehyde solution in PBS. FMOs controls were always included in the assays.

Samples were acquired on a flow cytometer (BD FACSCanto II) and data analyzed with the FACSDiva software (BD Biosciences).

Monocytes were identified by their Side/Forward scatter properties, excluding debris and gated as CD3-/CD45R/B220-/Ly-6G-/NK1.1-(Lineage-)/CD11b+ cells. Aβ uptake was assessed by reporting the percentage and Mean Fluorescence Intensity (GeoMean) of positive HiLyte Fluor™ 488 Aβ1-42 cells among gated monocytes.

### Examples 16-17

### Western Blot

For total Aβ detection, 10-30 μg of cell extracts were separated on a precast 10-20% SDS polyacrylamide Tris-Tricine gel (Bio-Rad, Canada). Separated proteins were then transferred onto polyvinylidene fluoride (PVDF) membranes (PerkinElmer, Canada). Membrane were incubated in DPBS (Gibco, USA) at 90° C. for 10 min. Membrane were then incubated for 1 hour in blocking solution, which consist in TBS-T (50 mM Tris-HCl pH 8.0; 150 mM NaCl; 0.05% Tween 20) supplemented with 1% BSA (Sigma, Canada) and 5% non-fat dry milk, and probed for 16 hours at 4° C. with a mouse anti-amyloid beta protein monoclonal antibody clone 6E10 (Covance, USA) diluted to 1:1000 in TBS-T supplemented with 5% BSA and 0.02% sodium azide. Membranes were washed once for 15 min and three times for 5 min in TBS-T at room temperature. Between each washing step, membranes were rinsed three times with TBS-T. Membranes were then incubated for 1 hour at room temperature in blocking solution supplemented with goat anti-mouse IgG HRP (Jackson, USA) diluted at 1:10000. Membranes were washed and rinsed as described above before being rinsed with four times with Milli-Q water. Localization of the HRP signal on the membrane was revealed using Super Signal West Dura Extended Duration Substrate (Thermo Scientific, USA) accordingly to the manufacturer instructions and revealed on Amersham Hyperfilm (GE, USA). To detect β-actin, membranes were stripped in Reblot Plus Strong solution (Millipore, USA) accordingly to the manufacturer recommendation and incubated in blocking solution for 1 hour. Membrane were then incubated for 16 hours at 4° C. in anti-8-actin rabbit monoclonal antibody (13E5, Cell Signaling Technology, USA) diluted at 1:2000 in TBS-T supplemented with 5% BSA and 0.02% sodium azide. Membranes were washed, incubated with goat anti-rabbit IgG HRP (H+L) (Jackson, USA) diluted at 1:5000 in blocking buffer, washed and revealed as described above. Films were digitalized using a HP Scanjet 4370 (HP) and signal intensity was measured using ImageJ (version 1.44p, NIH).

### Example 1

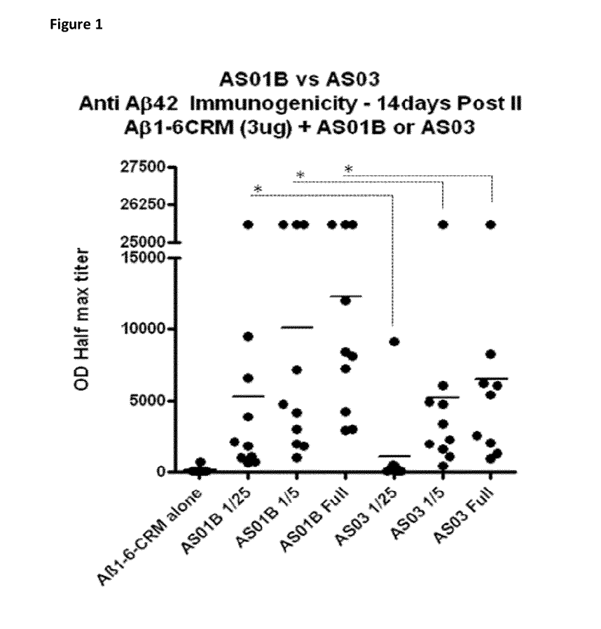

### FIG. 1

Anti-amyloid beta (1-42) antibodies were quantified in mice serum via ELISA, as described in the Materials and Methods. Seven groups of mice were used. All mice received 3 μg peptide content of AB1-6-CRM. Adjuvants used were: none, AS01B 1/25; AS01B ⅕; AS01B Full; AS03 1/25; AS03 ⅕; and AS03 Full. Anti-AB(1-42) antibody was measured using the method as described above.

Results are shown in FIG. 1: Higher anti Aβ42 specific immunogenicity is observed with AS01B compared to AS03 when used as an adjuvant in the presence of a stable dose of Aβ1-6 CRM conjugate (3 ug peptide content). Statistical comparison meaning is denoted (asterisk (*) indicates P less than 0.05) following ANOVA analysis of variance test combining with post-hoc Tukey-Kramer test.

### Example 2

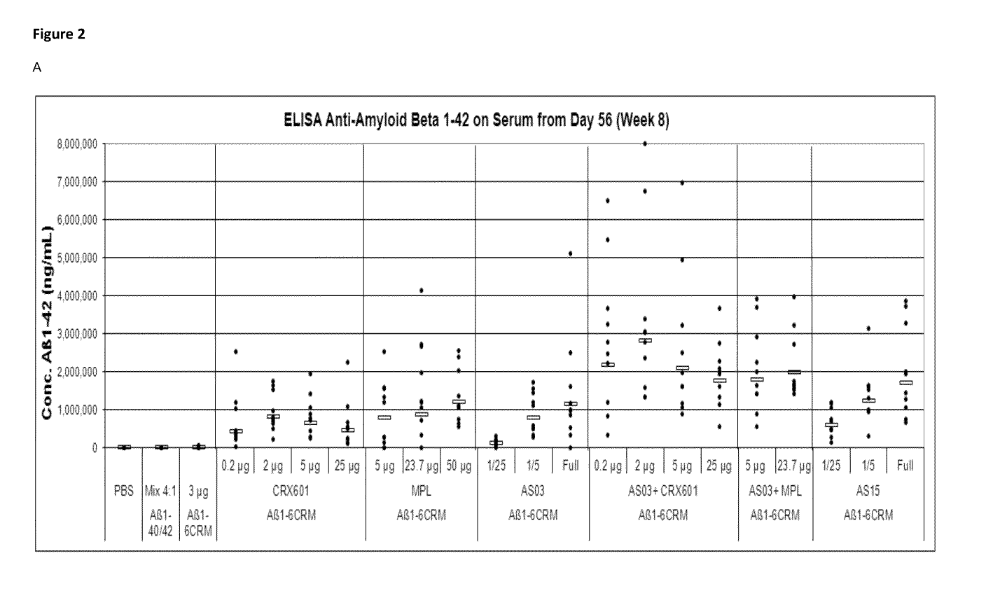

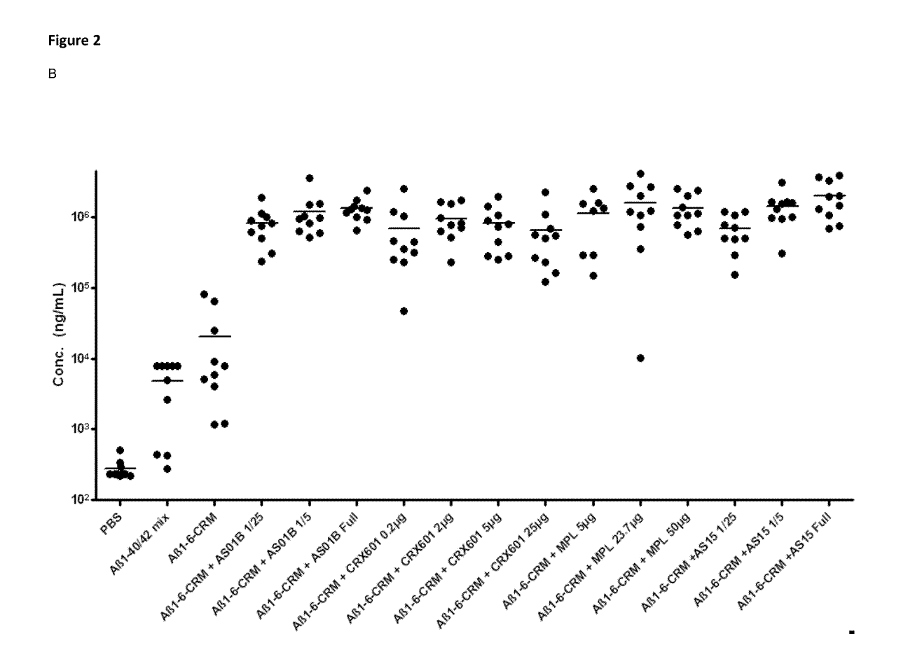

### FIG. 2

Antibodies generated to amyloid beta 1-42 were measured that were promoted by different doses of TLR containing adjuvants such as MPL or CRX601 (TLR4 agonistic ligands), AS15 (a TLR4 and TLR9 agonistic ligand) compared to appropriate controls (non adjuvanted Aβ40/42 alone at ratio 4:1), non adjuvanted Aβ1-6CRM alone or with water-oil emulsion (AS03) or in combination with AS03-CRX601.

Immunogenicity study in C57BL6 mice that have been immunized with different adjuvants combined with Aβ antigens (Aβ40+42 or Aβ1-6). Data showed the value of AS01B combined with Aβ1-6CRM compared to Aβ40+42 non-adjuvanted formulation or Aβ1-6CRM alone. The asterisk (*) denotes statistical difference p<0.05 using post-hoc analysis using comparison Dunnet test.

### Method:

The immunogenicity was performed using anti Aβ42 ELISA previously described. The mice were injected at day 1, 14 and 28. At day 56, terminal sera were used to measure the anti Aβ42 antibody level. The Aβ1-40/Aβ42 mix was prepared as previously described (Maier M, Seabrook T J, Lemere C A. Modulation of the humoral and cellular immune response in Abeta immunotherapy by the adjuvants monophosphoryl lipid A (MPL), cholera toxin B subunit (CTB) and E. coli enterotoxin LT(R192G). Vaccine. 2005. Oct. 25; 23(44):5149-59. PubMed PMID: 16054274.

Twenty-two groups of mice were used. As shown on FIG. 2 (from left to right) the treatments given were: PBS; Mix 4:1 AB1-40/42; 3 μg AB(1-6)-CRM; 3 μg AB(1-6)-CRM with 0.2 μg CRX601; 3 μg AB(1-6)-CRM with 2.0 μg CRX601; 3 μg AB(1-6)-CRM with 5.0 μg CRX601; 3 μg AB(1-6)-CRM with 25 μg CRX601; 3 μg AB(1-6)-CRM and 5.0 μg 3DMPL; 3 μg AB(1-6)-CRM and 23.7 μg MPL; 3 μg AB(1-6)-CRM and 50 μg MPL; 3 μg AB(1-6)-CRM and AS03 1/25; 3 μg AB(1-6)-CRM and AS03 ⅕; 3 μg AB(1-6)-CRM and AS03 full; 3 μg AB(1-6)-CRM and AS03 and 0.2 μg CRX601; 3 μg AB(1-6)-CRM and AS03 and 2.0 μg CRX601; 3 μg AB(1-6)-CRM and AS03 and 5.0 μg CRX601; 3 μg AB(1-6)-CRM and AS03 and 25 μg CRX601; 3 μg AB(1-6)-CRM and AS03 and 5.0 μg 3DMPL; 3 μg AB(1-6)-CRM and AS03 and 23.7 μg 3DMPL; 3 μg AB(1-6)-CRM and 1/25 AS15; 3 μg AB(1-6)-CRM and ⅕ AS15; 3 μg AB(1-6)-CRM and Full dose of AS15.

For the AS01B, AS03 and AS15. The mouse dose (full dose) is equal of the 1/10 human dose.

The mouse dose of AS01B contains 5 ug of MPL 3D co-formulated in neutral liposome, 5 ug of QS21. Those doses are per mouse and were injected using the intramuscular route (i.m.) 25 ul per mouse of AS01B+25 ul of PBS (phosphate buffer saline) or 25 ul of the appropriate peptide.

AS03: The mouse dose of AS03 contains 25 ug of SB62 tocopherol-base water-oil emulsion. The preparation of the SB62 emulsion is made by mixing under strong agitation of an oil phase composed of hydrophobic components (α-tocopherol and squalene) and an aqueous phase containing the water soluble components (Tween 80 and PBS mod (modified), pH 6.8). While stirring, the oil phase ( 1/10 total volume) is transferred to the aqueous phase ( 9/10 total volume), and the mixture is stirred for 15 minutes at room temperature. The resulting mixture then subjected to shear, impact and cavitation forces in the interaction chamber of a microfluidizer (15000 PSI-8 cycles) to produce submicron droplets (distribution between 100 and 200 nm). The resulting pH is between 6.8±0.1. The SB62 emulsion is then sterilised by filtration through a 0.22 μm membrane and the sterile bulk emulsion is stored refrigerated in Cupac containers at 2 to 8° C. Sterile inert gas (nitrogen or argon) is flushed into the dead volume of the SB62 emulsion final bulk container for at least 15 seconds. The final composition of the SB62 emulsion is as follows: Tween 80:1.8% (v/v) 19.4 mg/ml; Squalene: 5% (v/v) 42.8 mg/ml; α-tocopherol: 5% (v/v) 47.5 mg/ml; PBS-mod: NaCl 121 mM, KCl 2.38 mM, Na2HPO4 7.14 mM, KH2PO4 1.3 mM; pH 6.8±0.1.25 ul per mouse of the AS03 was used per mouse.

CRX601/AS03: 2 μg of CRX601 was diluted in PBS for a final volume of 25 μl. The 25 μl of CRX601 was added slowly to the 25 μl AS03 (2×concentrated). The vaccine was mixed by magnetic stirring at medium speed. Formulation was performed extemporaneously, the injections occurred within 60 min following the end of the formulation.

AS15: As described WO 00/62800. AS15 is a combination of the two adjuvant systems, AS01B and AS07A. AS07A is composed of CpG 7909 (also known as CpG 2006) in phosphate buffer saline.

### Formulations:

Formulations were performed the days of injections. The volume of injection for one mouse was 50 μl. A typical formulation contains as follows: 20 μg-25 μg antigen was diluted with H₂O and PBS pH 7.4 for isotonicity.

Results are shown in FIG. 2, where the Y axis is concentration of anti-Aβ(1-42) antibodies in ng/ml: Higher anti Aβ42 specific immunogenicity was observed with CRX601 co-formulated with AS03 adjuvant compared non-adjuvant control or AS03 adjuvanted peptide. Statistical comparison meaning is denoted (asterisk (*) indicates P less than 0.05) following ANOVA analysis of variance test combining with post-hoc Tukey-Kramer test.

### Example 3

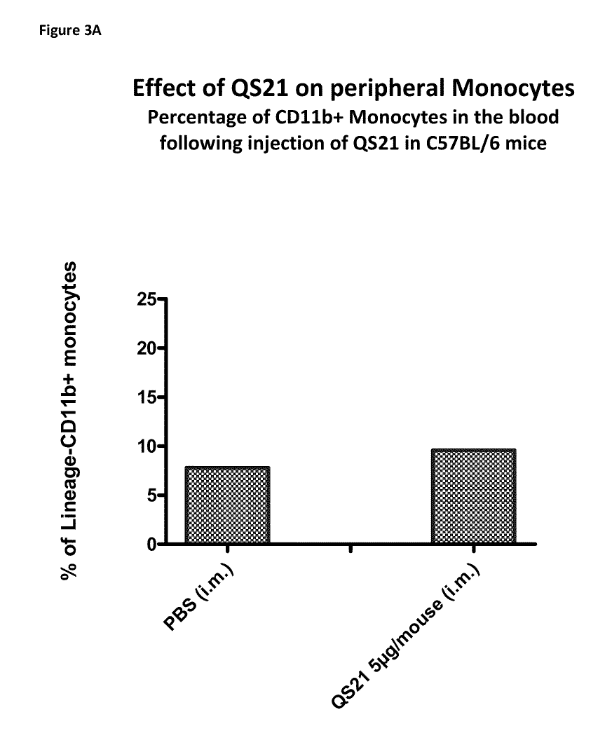

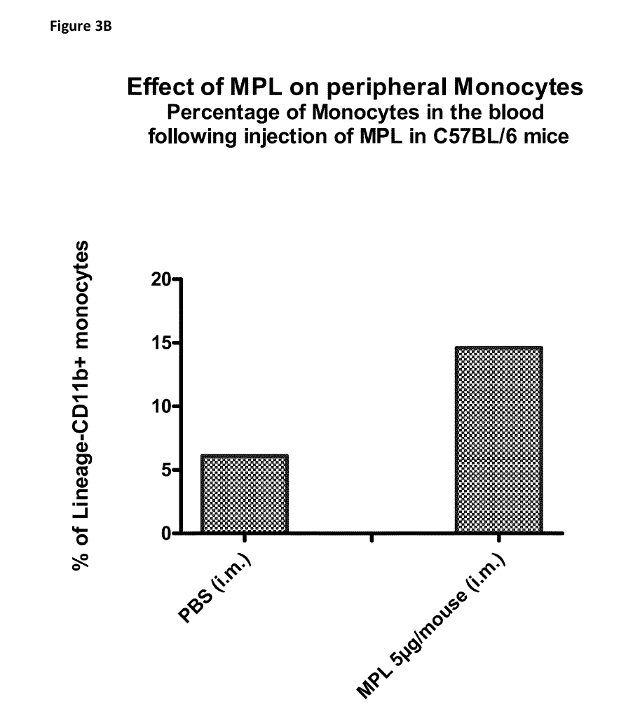

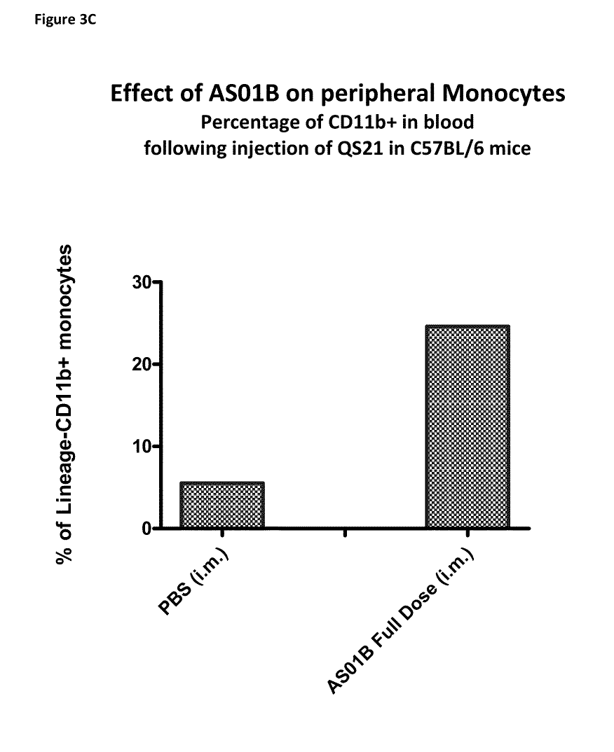

### FIG. 3

Monocyte analysis and counting was performed as described above and provided the percentage of Lineage-CD11b+ monocytes (Y axis of FIG. 3). Six groups of C57BL/6 mice were used. The treatments were: PBS (i.m.); QS21 5 ug (i.m); 3DMPL 5 ug (i.m.); and AS01B Full Dose (i.m).

Results are shown in FIGS. 3A, 3B and 3C, where the left-hand bar in each graph is a PBS treatment group.

Different compositions were used to test for stimulation of peripheral monocytes.

The number of monocytes is up-regulated 4.5 fold by AS01B. AS01B resulted in a greater increase in monocytes than 3D MPL.

### Example 4

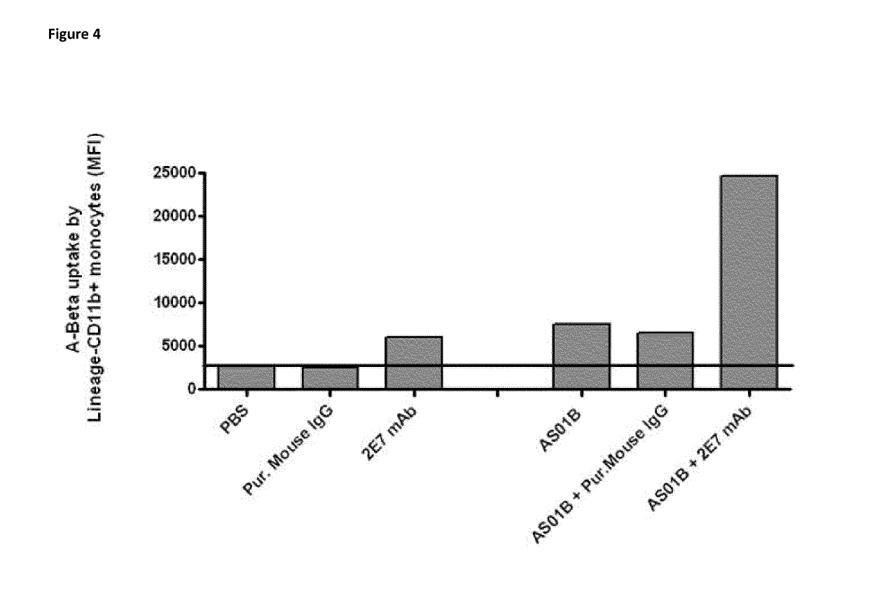

### FIG. 4

The combination of TLR4 agonist and Aβ42 specific antibodies synergistically up-regulates the Abeta phagocytosis.

The experiment looked at ex vivo uptake of Aβ 1-42 HiLyte Fluor 488 within 2 hours by CD11 b+ peripheral blood monocytes; The PBMCs were prepared from C57Bl/6 mice following injection of AS01B or PBS as control, then, pre incubated in vitro, with a mouse monoclonal anti Aβ 2E7 or irrelevant mouse IgG as control.

A flow cytometry analysis shows a higher uptake of the Aβ by CD11b+ cells from mice treated with AS01B than those who received PBS; In addition the presence of the ABeta-specific mAb increase the uptake of ABeta compared to IgG control, The uptake was synergistically enhanced when the mAb 2E7 was combined with monocytes from AS01B-treated mice compared to PBS control groups. Monocytes coming from AS01B injected animals had an increased activity of Aβ phagocytosis. The phagocytosis was promoted when a specific Aβ antibody is used. The Aβ phagocytic activity was up-regulated following an incubation with Aβ specific antibody such as 2E7 GSK antibody (an IgG2a) mouse version.

The combination of TLR4 agonist and Aβ specific antibodies up-regulated the Aβ phagocytosis more than each agent separately (synergy).

### Example 5

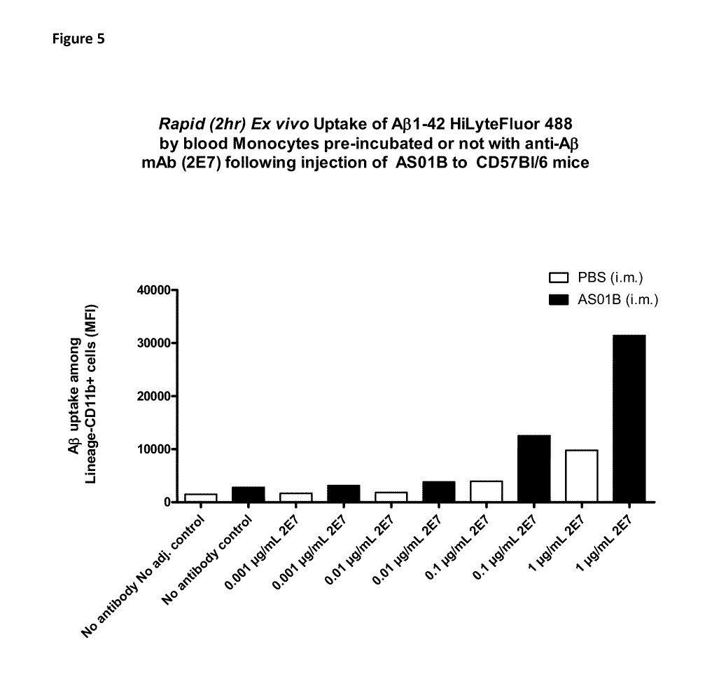

### FIG. 5

Ex vivo uptake of Aβ 1-42 HiLyte Fluor 488 pre incubated, or not, with anti Aβ 2E7 or polyclonal anti Aβ 1-6 (mouse sera) by blood live monocytes obtained from WT CD57Bl/6 mice following a single i.m. injection of AS01B (2 hr incubation with Aβ+/−antibody)

On FIG. 5, white bars represent mice receiving PBS (adjuvant control); black bars represent AS01B treatment. Treatment groups, as shown left to right on FIG. 5, were: Control (no AS01B, no antibody preincubation); AS01B injection only (no antibody preincubation); PBS plus preincubation with 0.001 ug/ml 2E7; AS01B plus preincubation with 0.001 ug/ml 2E7; PBS plus preincubation with 0.01 ug/ml 2E7; AS01B plus preincubation with 0.01 ug/ml 2E7; PBS plus preincubation with 0.1 ug/ml 2E7; AS01B plus preincubation with 0.1 ug/ml 2E7; PBS plus preincubation with 1.0 ug/ml 2E7; AS01B plus preincubation with 1.0 ug/ml 2E7.

As shown on FIG. 5, Preincubation with 1.0 ug/mL 2E7 resulted in a 3.2 fold increase in uptake in monocytes from mice receiving AS01B, compared to mice receiving PBS. In monocytes from mice who received AS01B, preincubation with 1 ug/ml of 2E7 resulted in in a ten-fold increase in uptake (compared to no preincubation). There was an 18.5-fold increase in uptake in monocytes from mice who received PBS (no preincubation) to mice who received AS01B (with preincubation).

Herein, we have developed a flow cytometry readout to measure the efficacy of the Aβ-specific antibodies from adjuvanted peptide immunization. Monocytes coming from AS01B-injected animals had an increased activity of Aβ phagocytosis. The phagocytosis is promoted when a specific Aβ antibody is used. This phagocytic activity is up-regulated following an incubation with Aβ specific antibody such as 2E7 GSK antibody (IgG2a) mouse version. This Aβ uptake phenomenon promoted by antibody and adjuvant could be observed starting at the 0.1 ug/ml dose of anti Aβ-specific antibody.

### Example 6

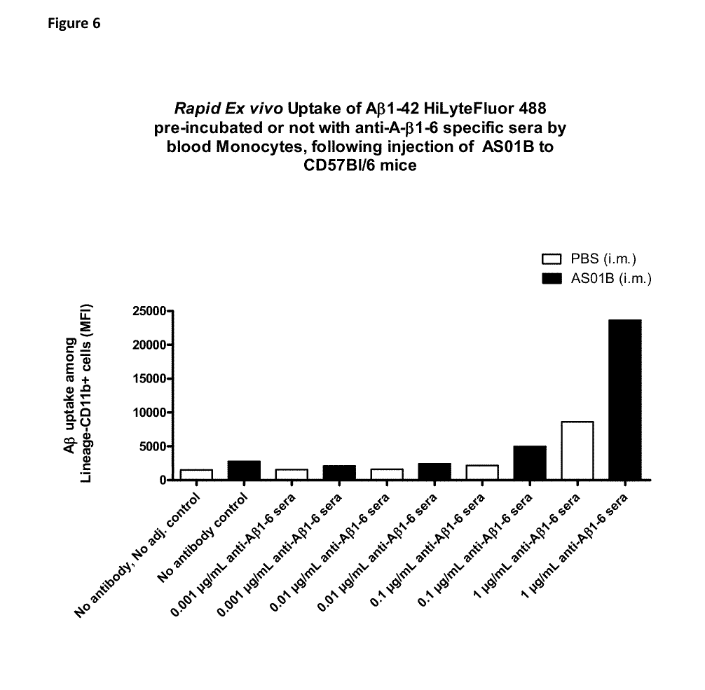

### FIG. 6

Ex vivo uptake of Aβ 1-42 HiLyte Fluor 488 pre incubated or not with polyclonal anti Aβ 1-6 antibodies (mouse sera) by blood live monocytes, obtained following a single injection of AS01B to WT CD57Bl/6 mice (2 hr incubation with fluorescent Aβ1-42+/−anti Aβ1-6 sera).

On FIG. 6, white bars represent mice receiving PBS (adjuvant control); black bars represent AS01B treatment. As shown on FIG. 6, Preincubation with 1.0 ug/mL anti-AB1-6 resulted in a 2.7 fold increase in uptake in monocytes from mice receiving AS01B, compared to mice receiving PBS. In monocytes from mice who received AS01B, preincubation with 1 ug/ml of antibody resulted in in an 8.5-fold increase in uptake (compared to no preincubation). There was a 15.6 fold increase in uptake in monocytes from mice who received PBS (no preincubation) compared to mice who received AS01B (with preincubation).

We have developed a flow cytometry readout to measure the efficacy of the Aβ-specific antibodies from adjuvanted immunization. Monocytes coming from AS01B-injected animals had an increased activity of AB phagocytosis. The phagocytosis was promoted when Aβ1-6 specific sera were used. This Aβ uptake phenomenon promoted by antibody and adjuvant could be observed starting at low the 0.1 ug/ml dose of anti Aβ-specific antibody.

### Example 7

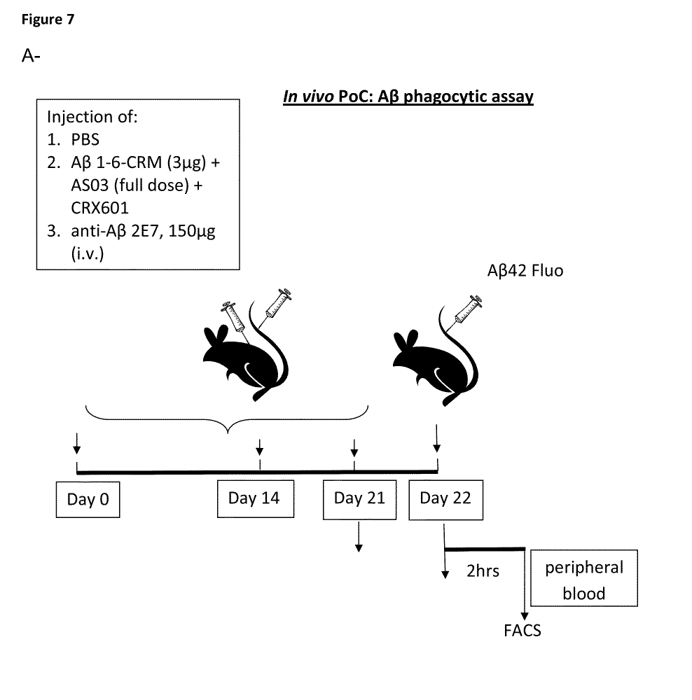

### FIG. 7

In vivo Phagocytosis assay of Aβ 1-42 by CD11 b+ monocytes in mice.

We demonstrate that the Aβ uptake in peripheral blood is promoted by antibody from both active and passive immunization. In addition, we demonstrate that the active immunization by the combination of ABeta antigen+AS01B can be more efficient than passive immunization

The immunization schedule as shown in FIG. 7A and the method as described above was used. FIG. 7B shows that the % of HiLyte Fluor 488 positive monocytes (indicating phagocytosis of Aβ 1-42) was higher in the active immunization group, (Aβ1-6 CRM+AS03/CRX601, a synthetic TLR4 co-formulated with AS03 emulsion) compared to the PBS control or the passive immunization (intravenous injection of anti Aβ monoclonal antibody (2E7)).

### Example 8

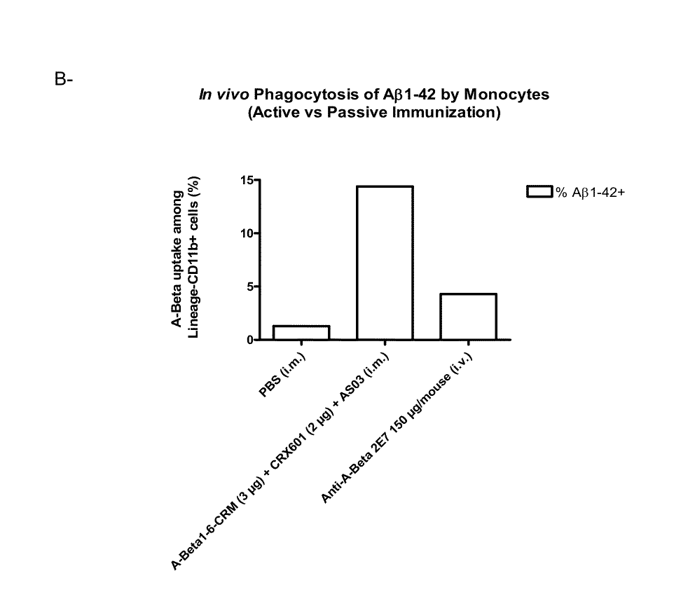

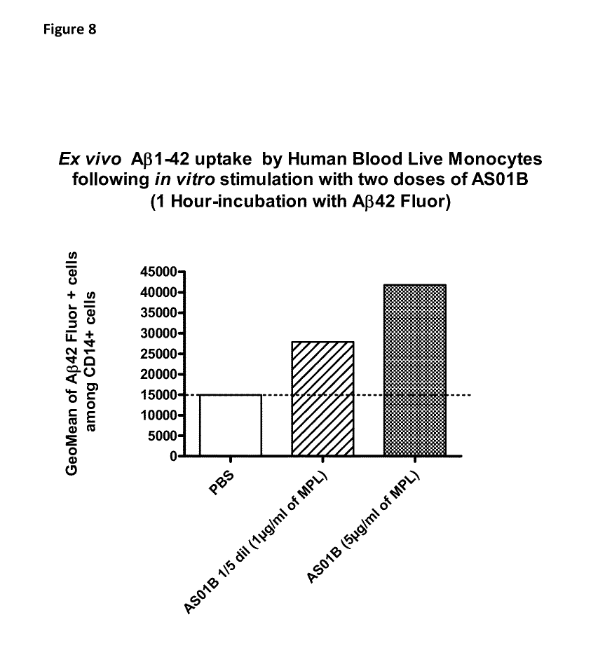

### FIG. 8

Live human peripheral blood monocytes (CD14+ cells) were isolated from whole blood and treated in vitro with: PBS (control); AS01B ⅕ (1 μg/ml of 3D MPL); or AS01B (5 μg/ml of 3D MPL). Cells were incubated for one hour with HiLyte Fluor 488 AB1-42. FIG. 8 shows the Mean Fluorescence Intensity (GeoMean) of positive HiLyte fluor488 Aβ1-42 cells among the CD14+ cells, which provides a measure of Ab uptake. Uptake was increased by the presence of 3D MPL.

In the present invention we have shown an increasing Aβ42 uptake in peripheral human monocytes (CD14+ cells) after whole blood in vitro stimulation with AS01B at dilution ⅕ (1 ug of MPL per ml) or AS01B (5 ug per ml of MPL) compared to non-stimulated whole blood (PBS).

### Example 9

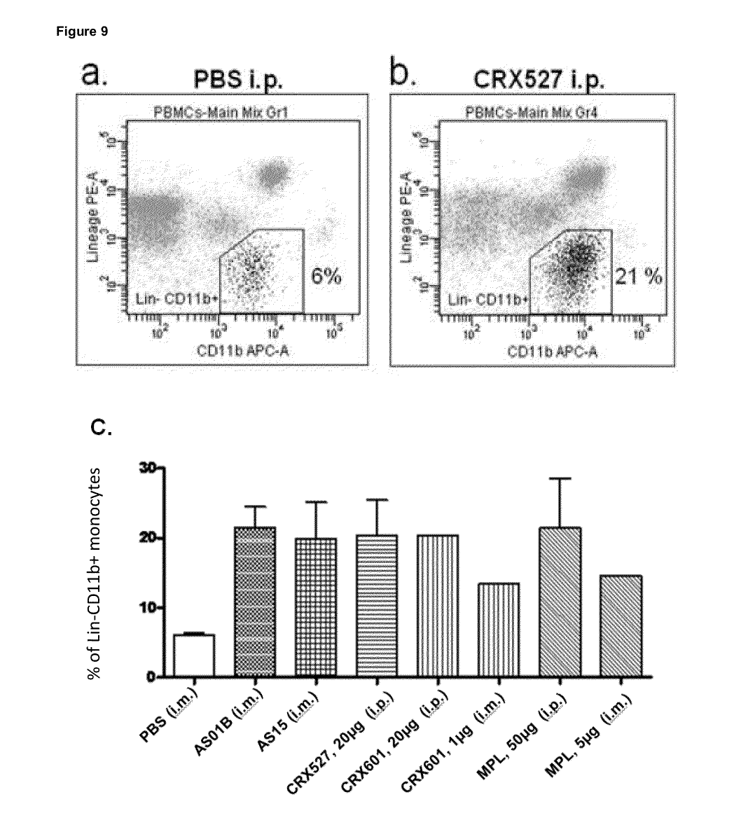

### FIG. 9

Upregulation of circulating monocytes numbers following the injection of compositions comprising TLR4 agonists such as 3D MPL, AS01B, AS15, CRX527 or CRX601 via the i.m. or i.p. routes.

Eight groups of C57BL/6 mice were treated with the following: PBS i.m. (control); AS01B Full Dose (i.m.); AS15 Full Dose i.m.; CRX527 (20 ug/mouse i.p.); CRX601 (20 ug/mouse i.p.); CRX601 (1 ug/mouse i.m.); 3D MPL (50 ug/mouse, i.p.); 3D MPL (5 ug/mouse, i.m.). Monocyte analysis was conducted as described in the Methods and Materials, above, to measure the percentage of lineage CD11b+ monocytes in peripheral blood. As shown in FIG. 9, the 5 ug dose of 3D MPL resulted in about double the number of CD11b+ monocytes measured compared to the PBS control.

### Example 10

### FIG. 10

A single intramuscular injection of different doses of 3D MPL (5 μg, 25 μg and 50 μg) was carried out. 3DMPL was able to stimulate monocyte numbers at 5 and 25 ug/mouse dose.

Four groups of C57BL/6 mice were treated with the following (single i.m. injection): PBS i.m. (control); 3D MPL (5 ug/mouse, i.m.); 3D MPL (25 ug/mouse, i.m.); and 3D MPL (50 ug/mouse, i.m.). Monocyte analysis was conducted as described in the Methods and Materials, above, to measure the percentage of lineage CD11b+ monocytes in peripheral blood. Results are shown in FIG. 10.

### Example 11

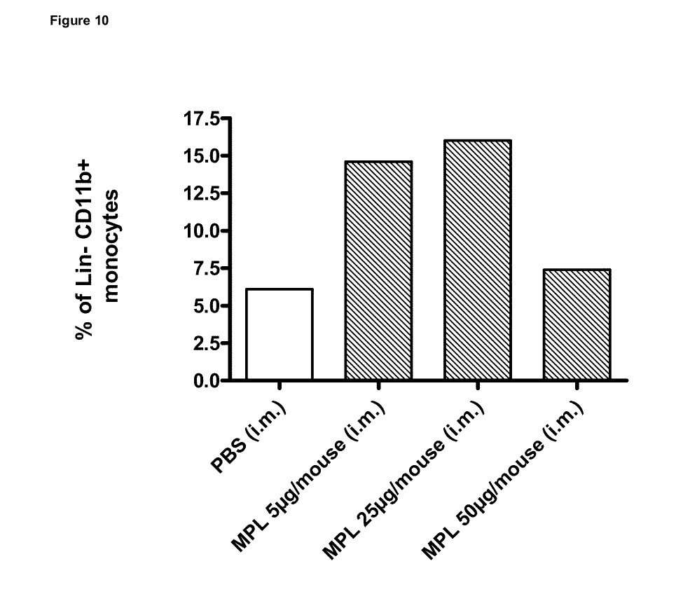

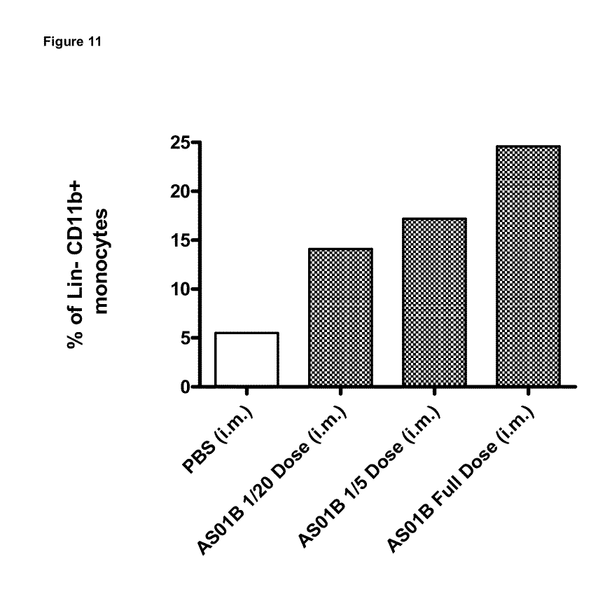

### FIG. 11

A single intramuscular injection of different doses of AS01B ( 1/20 vs ⅕ vs mouse full dose) was carried out. Four groups of C57BL/6 mice were treated with the following (single i.m. injection): PBS i.m. (control); AS01B 1/20 dose; AS01B ⅕ dose; and AS01B full mouse dose. Monocyte analysis was conducted as described in the Methods and Materials, above, to measure the percentage of lineage CD11b+ monocytes in peripheral blood. Results are shown in FIG. 11.

A dilution of 1/20 of AS01B was enough to trigger an increase of the monocyte count within the peripheral blood compared to PBS. A constant increase is noted until the mouse full dose, i.e. the AS01B mouse full dose is containing 5 μg of 3D MPL and 5 μg of QS21.

### Example 12

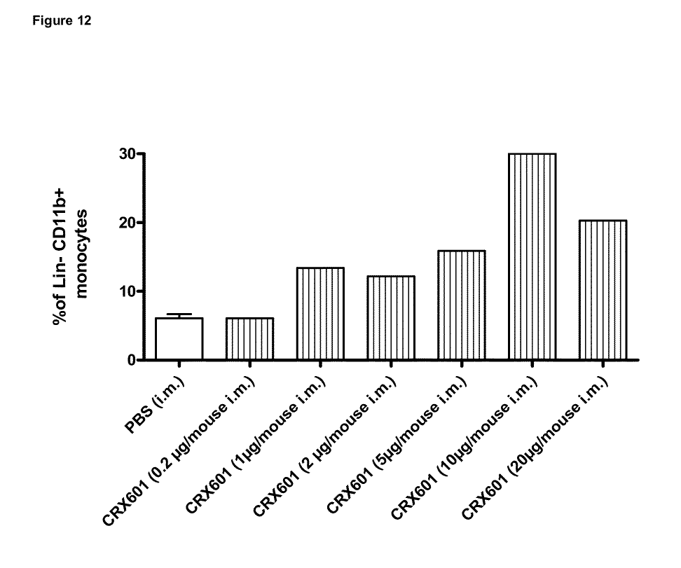

### FIG. 12

TLR4 agonists free of endotoxin trigger a higher number of CD11 b+ monocytes within the periphery.

Seven groups of C57BL/6 mice were treated with the following (single i.m. injection): PBS (control); CRX601 (0.2 ug/mouse); CRX601 (1 ug/mouse); CRX601 (2 ug/mouse); CRX601 (5 ug/mouse); CRX601 (10 ug/mouse); CRX601 (20 ug/mouse). Monocyte analysis was conducted as described in the Methods and Materials, above, to measure the percentage of lineage CD11b+ monocytes in peripheral blood. Results are shown in FIG. 12.

We have performed a dilution analysis of CRX601 to identify the dose of CRX601 triggering the CD11b+ monocyte number. As shown in FIG. 12, 1 μg of CRX601 is enough to trigger an increase of the monocyte count within the peripheral blood, compared to PBS. The maximum response was observed at 10 μg dose and the effect is down modulated at 20 μg dose.

### Example 13

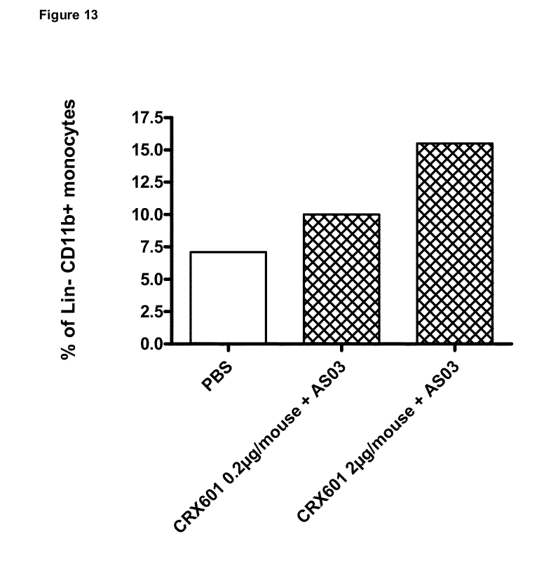

### FIG. 13

We performed a dilution analysis of CRX601 combined with a constant dose of AS03. The CRX601/AS03 combination induced a strong antibody response as shown as in FIG. 2. Peripheral blood monocyte numbers were measured following a single intramuscular injection of different doses of CRX601 (0.2 μg and 2 μg). Both doses demonstrated an increase in monocyte numbers when compared with PBS.

Three groups of C57BL/6 mice were treated with the following (single i.m. injection): PBS (control); CRX601 (0.2 ug/mouse)+AS03; CRX601 (2 ug/mouse)+AS03. Monocyte analysis was conducted as described in the Methods and Materials, above, to measure the percentage of lineage CD11b+ monocytes in peripheral blood. Results are shown in FIG. 13.

### Example 14

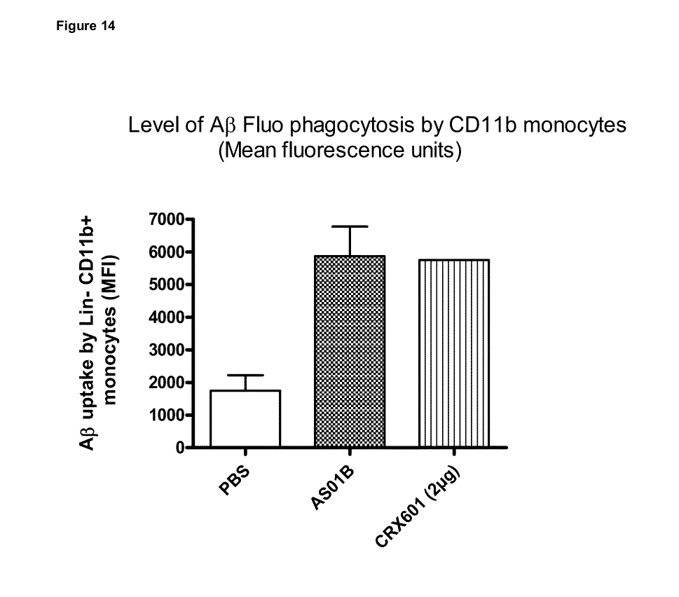

### FIG. 14

To examine the function of the increase of monocytes in the peripheral blood, we examined the capacity of those monocytes to uptake Aβ42 in a test tube. In order to measure that phagocytic activity, we used fluorescent HiLyteFluo Aβ42 (Anaspec Inc). Three groups of C57BL/6 mice were treated with the following: PBS i.m.; AS01B i.m. (5 ug/mouse); CRX601 i.m. (2 ug/mouse). Isolation of monocytes and ex vivo uptake of Ab1-42 peptide by peripheral blood lineage CD11 b+ monocytes was assessed as set forth in Methods and Materials, above. Results are shown in FIG. 14. Flow cytometry analysis demonstrated that the intramuscular injection of AS01B (mouse full dose, 5 ug per mouse) or CRX601 (2 μg dose) trigger the monocytes to be able to uptake an higher amount of Aβ42 compared to a non adjuvanted mouse monocytes (PBS group).

### Example 15

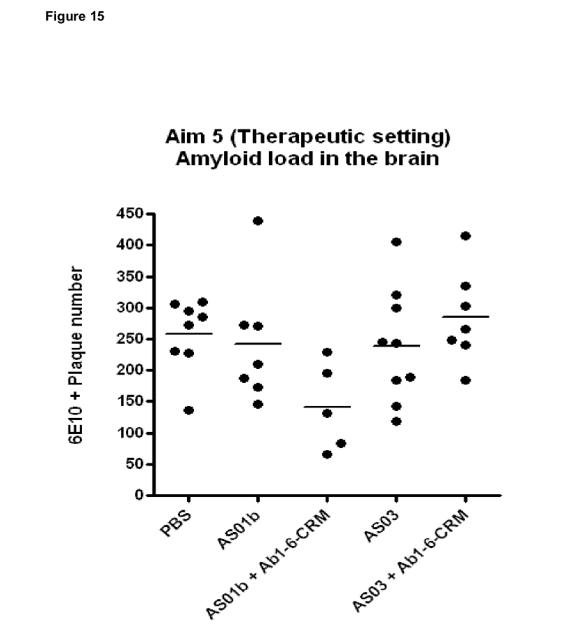

### FIG. 15

Injection of AS01B or AS03+/−Aβ(1-6)CRM Peptide and Effect on Aβload in the Brain

All immunogens were injected using the intramuscular route. Six bi-weekly injections were performed in APP-PS1 mouse model (Savonenko A 2005. Neurobiol Dis 18(3):602-17), mice being 6 months old at the beginning of the treatment. Treatment groups were the following:

| | | Dose/ | | | |
|---|---|---|---|---|---|
| | | peptide/ | | Volume | |
| | | Injection | Dose/adjuvant/ | injection | |
| Group | Immunogen | (μg) | injection | (μL) | route |
| 1 | PBS | — | — | 50 | i.m. |
| 2 | AS01B | — | 1/10 of human | 50 | i.m. |
| | | | dose | | |
| 3 | AS01B + Aβ1- | 3 | 1/10 of human | 200 | i.m. |
| | 6CRM1 | | dose | | |
| 4 | AS03 | — | 1/10 of human | 50 | i.m. |
| | | | dose | | |
| 5 | AS03 + Aβ1- | 3 | 1/10 of human | 50 | i.m. |
| | 6CRM1 | | dose | | |

To count Aβ plaques, sections of APP_{Swe}/PS1 mice were immunostained for Aβ (polyclonal mouse anti-Aβ 6E10, 1:3000; Covance) as previously reported (Richard et al., 2008). Two sections were chosen for caudal cortex area and two sections for rostral cortex area. Unbiased stereological analysis was performed as described previously (Richard et al., 2008). Briefly, the contours of the cortex areas were traced as virtual overlay on the steamed images and areas were calculated. The area occupied by all A β̅labeled plaques was determined in each structure. The stereo Investigator software (MicroBrightField) sequentially chose counting frames (350×350 μm) every 700 μm in the x axis and every 700 μm in the y axis while moving automatically the motorized stage into the previously delimited zones in the cortex. Analyzed areas of structures were calculated with the NeuroExplorer software (MicroBrightField). All parameters analyzed were reported to the whole section.

Aβ total plaque loading analyses reveal a tendency of lower amyloid plaque burden between the PBS control group and AS01B+Aβ1-6 CRM group (student t-test). Mouse brain sections were stained using a primary antibody directed against Aβ(6E10) tagged with an Cy3-conjugated secondary antibody. Number of Aβ plaques per 6 cross-sections of half brain were determined by unbiased stereology in the cortex of immunized transgenic APPswe/PS1 mice.

### Example 16

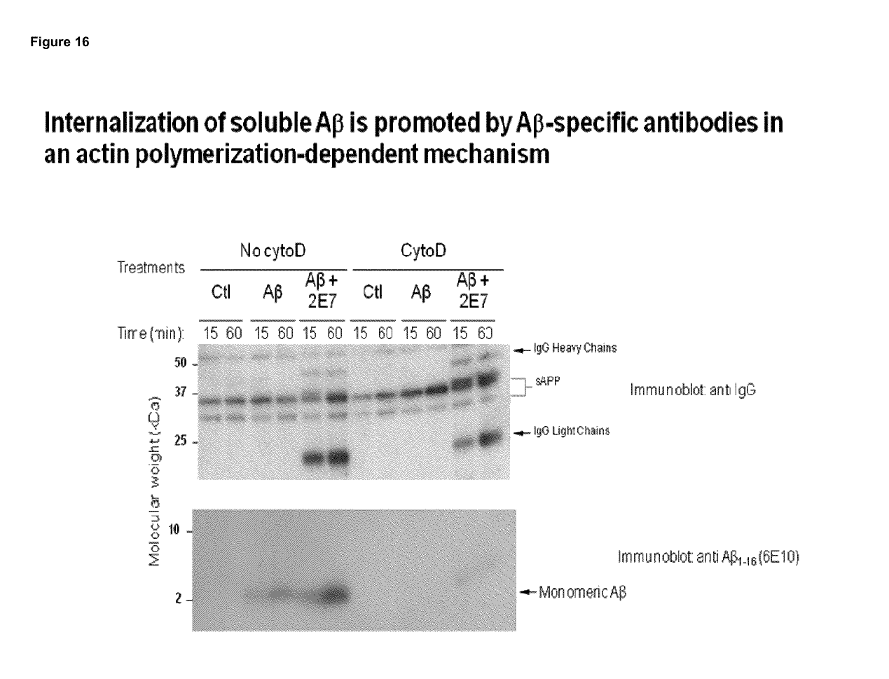

### FIG. 16

Aβ specific antibodies stimulate the uptake of soluble Aβ₁₋₄₂ toxic peptides in an actin polymerization-dependent mechanism. The mouse microglia cell line BV2 was incubated for 30 min under control condition or with cytochalasin D, an inhibitor of actin polymerization, before the addition of vehicle media or media supplemented with Aβ₁₋₄₂, or Aβ₁₋₄₂ in presence of 2E7 anti-Aβ antibodies. Cells were incubated for the indicated period of time, resuspended by trypsinization, collected, solubilised in lysis buffer and further analyzed by immunoblotting. Immunoblot using anti-IgG (upper panel) demonstrate that cells cultured in presence of 2E7 antibody exclusively contained IgG light and heavy chains. The presence of 2E7 anti-Aβ antibodies drastically increased the cellular uptake of Aβ₁₋₄₂, a phenomenon further inhibited by the presence of cytochalasin D (immunoblot using anti Aβ₁₋₁₆ (6E10) antibodies, bottom panel).

### In Vitro Phagocytosis in BV2 Cell:

BV2 microglia cells were seeded at 900 000 cells/60 mm cell culture Petri dish 24 hours prior the experiment, and cultured in complete media (Dulbecco's Modified Eagle Medium (DMEM, Gibco, USA) supplemented with 10% fetal bovine serum (FBS) (Gibco, USA), 1 mM sodium pyruvate (Gibco, USA) and 100 U/mL of penicillin and 100 μg/mL of streptomycin (Gibco, USA)). Cells were washed with 3 mL DPBS (Gibco, USA) and incubated for 30 min in complete media supplemented or not with 5 μg/mL of cytochalasin D (CytoD) (Calbiochem, USA). Cells were then incubated for an additional 15 or 60 minutes with complete media supplemented or not with one of the following combination: 1 μg/mL Aβ₁₋₄₂, or 1 μg/mL Aβ₁₋₄₂ in presence of 1 μg/mL 2E7 anti-Aβ antibodies, 5 μg/mL CytoD, 5 μg/mL CytoD with 1 μg/mL Aβ₁₋₄₂, or 5 μg/mL CytoD with 1 μg/mL Aβ₁₋₄₂ and 1 μg/mL 2E7 anti-Aβ antibodies. Following incubation, remove media, rinse with 2.5 mL DPBS, add 0.4 mL 0.05% trypsin-EDTA (Gibco, USA), incubate at 37° C. for 2 min, shake the Petri dish to make sure that all cells detached, harvest floating cells with 3 mL complete media and transfer to a falcon tube. Rinse the Petri dish a second time with 3 mL complete media to collect all remaining cells. Centrifuge tubes for 3 min at 2000 RPM, discard the supernatant and wash the cell pellet with 3 mL ice cold DPBS. Centrifuge tubes for 3 min at 2000 RPM, remove supernatant, resuspend cell pellet in lysis buffer (100 mM Tris pH 7.4, 5 mM EDTA, 150 mM NaCl, 1% Triton X-100, 0.1% SDS, 50 mM NaF, 1 mM Na₃VO₄ and Complete protease inhibitor), incubate on ice for 10 min, transfer to a 1.5 mL eppendorf tube, centrifuge for 10 min at 4° C. at 14000 RPM and keep supernatant. Protein concentration in supernatant or cell extracts was then measured using the Bradford method (Coomassie Plus, The better Bradford assay reagent, Thermoscientific, USA) accordingly to the manufacturer recommendation and equal amount of protein were then analyzed by Western blot.

### Example 17

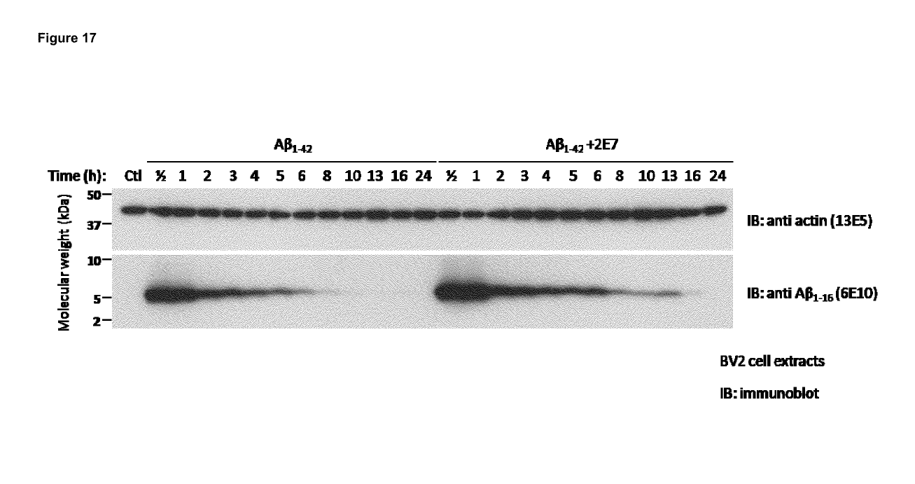

### FIG. 17

Aβ-specific antibody stimulates the uptake of Aβ1-42 by BV2 microglia, a process leading to the degradation of this toxic peptide.

BV2 microglia cells were incubated for 30 min under control condition, or with Aβ1-42, or with Aβ1-42 in presence of anti-Aβ antibodies (2E7). Cells were then washed with DPBS, and further incubated in fresh media (not containing Aβ1-42 peptides, nor 2E7 antibodies) for the indicated time period. Following incubation, cells were washed with ice-cold DPBS, solubilised in lysis buffer and further analyzed by immunoblotting. Immunoblot using anti-actin (13E5, Sigma Aldrich, Canada) (upper panel) demonstrates that cellular content in actin remains stable over the course of the experiment. Following 30 min incubation, BV2 cells had already absorbed detectable levels of Aβ1-42 peptides. In presence of 2E7 antibodies, BV2 cells absorb higher levels of Aβ1-42 peptides. The amount of intact Aβ peptides diminishes over time, which would correlate with its degradation. These data suggest that microglia are able to engulf and degrade toxic Aβ1-42 and that the amount of processed peptide is further amplified by the presence of an antibody directed against it.

### Phagocytosis In Vitro and Cell Lysate

BV2 microglia cells were seeded at 900 000 cells/60 mm cell culture Petri dish 24 hours prior the experiment, and cultured in complete media. Cells were washed with 3 mL DPBS (Gibco, USA) and incubated for 30 min in complete media supplemented or not with 1 μg/mL Aβ₁₋₄₂ or with 1 μg/mL Aβ₁₋₄₂ in presence of 1 μg/mL 2E7 anti-Aβ antibodies. Following the incubation, remove the media, gently add 3 mL PBS to rinse the cells, replace it by 3 mL of complete media free of Aβ₁₋₄₂ or 2E7, and incubate for additional periods of time (30 min, 1 h, 2 h, 3 h, 4 h, 5 h, 6 h, 8 h, 10 h, 13 h, 16 h and 24 h). At the end of each time point, remove media, rinse cells with ice-cold DPBS, add lysis buffer (100 mM Tris pH 7.4, 5 mM EDTA, 150 mM NaCl, 1% Triton X-100, 0.1% SDS, 50 mM NaF, 1 mM Na₃VO₄ and Complete protease inhibitor), collect cell extract using a cell scraper, transfer to a 1.5 mL eppendorf tube, incubate on ice for 10 min, centrifuge for 10 min at 4° C. at 14000 RPM and keep supernatant or cell extracts for following analysis. Protein concentration in cell extracts was measured using the Bradford method (Coomassie Plus, The better Bradford assay reagent, Thermoscientific, USA) accordingly to the manufacturer instructions and equal amount of protein were then analyzed by Western blot.

### Example 18

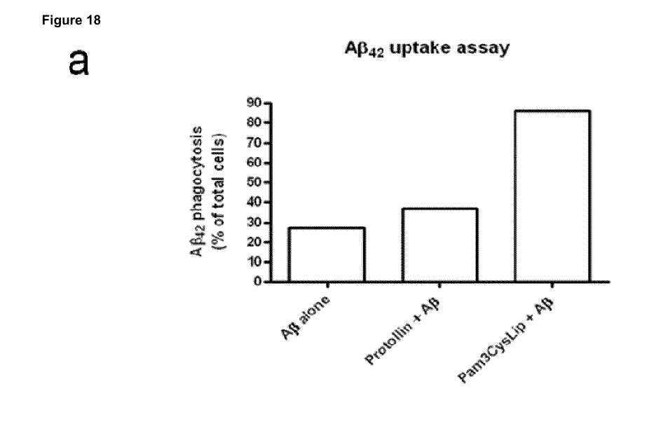

### FIG. 18

Phagocytosis of beta-amyloid 1-42 peptide by the human microglial cell line CHME was observed after the addition of TLR2 agonist containing adjuvants Protollin and Pam3CysLip peptide. In this experiment, the highest increase of phagocytosis (86% of cells) was observed with the purified Pam3CysLip peptide from Neisseria meningitidis strain 8047. The synthetic peptide sequence of the Pam3Cys Lip peptide is described herein: Pam3Cys—SQ EPAAPAAEAT PAAEAP. Protollin was used at concentration 1 ug per ml based on the LPS content from Shigella flexneri. Pam3Cys lip peptide was used at concentration of 5 ug per ml of the pure tri-palmitoylated peptide.

As shown in FIG. 18, phagocytosis of Aβ1-42 by human microglia cell line (CHME) is increased by the pre-incubation (18 hrs) of the cells with these TLR2 adjuvants in the presence of Abeta. Additionally, cells were imaged using 42 HiLyte 488 Fluo (Anaspec, Inc.) at 2 ug per ml in DMEM complete media (Invitrogen). Lysotracker red staining was performed and slides were mounted and co-stained with DAPI to show the nucleus (blue). Fluorescence microscopy of human microglia cell line showed the localization of Abeta1-42 within the lysosome after each treatment (results not shown). Lysotracker red reagent was purchased from Invitrogen and used as manufacturer recommendations.

Human microglia cell line CHME had a higher amyloid phagocytic activity following stimulation with TLR2 pure agonist Pam3CysLip peptide compared to PBS and to medium extend Protollin, which was previously shown to increase the phagocytic activity of Abeta1-42 (Hjorth et al Int J Alzheimers Dis. 2010 May 20; 2010. pii: 798424).

### Example 19

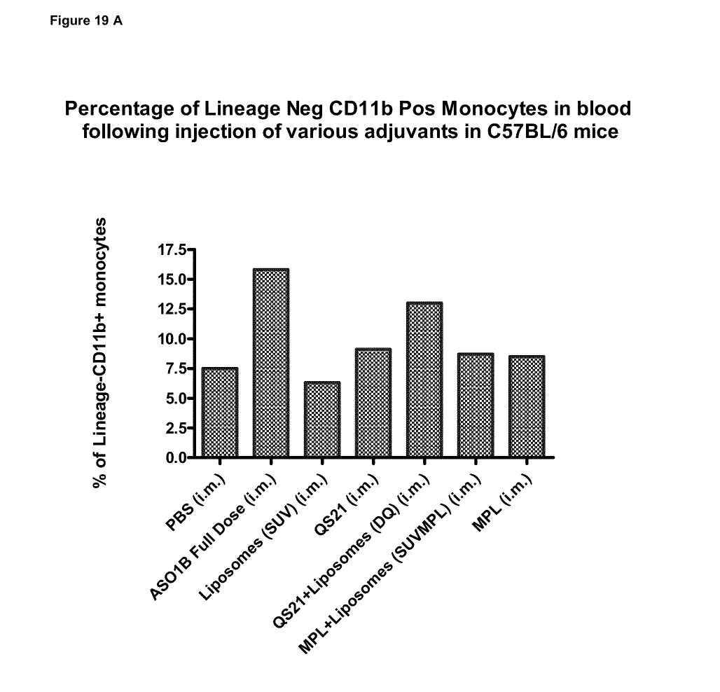

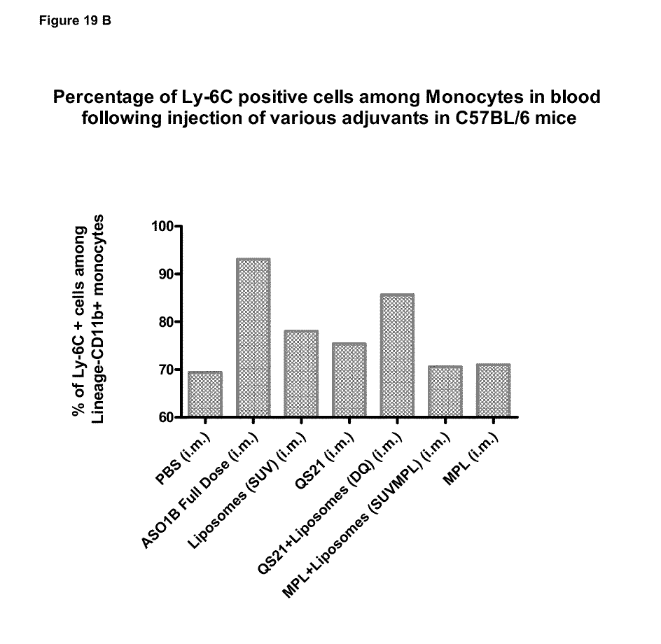

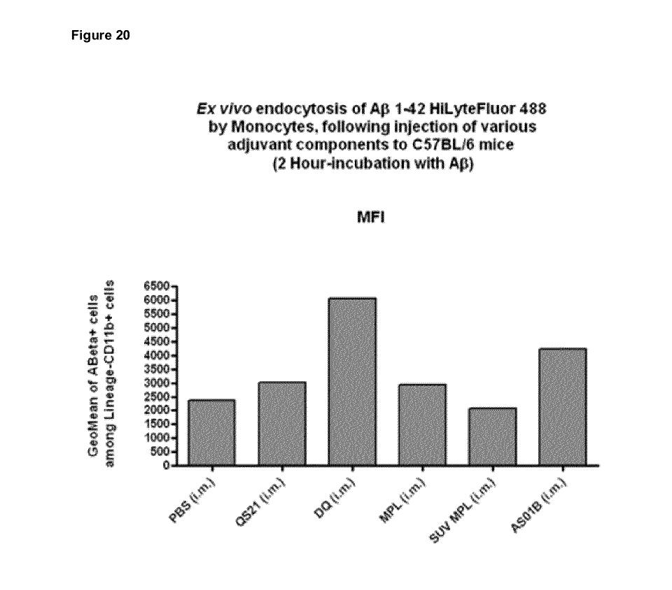

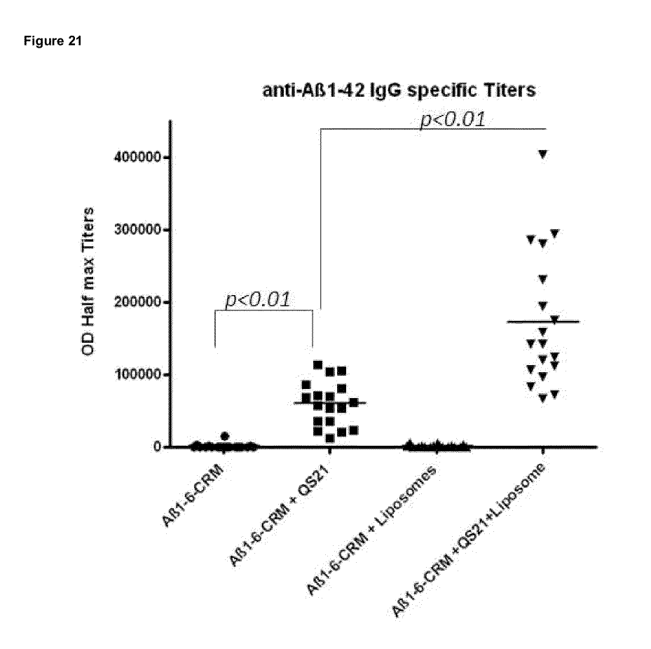

FIGS. 19-21, demonstrate that QS21 combined with liposomes in AS01B stimulates monocytes production (FIG. 19) and amyloid β uptake activity (FIG. 20).

### Materials and Methods

Aβ1-6 peptide was purchased from 21^{st} Century Bio as a TFA salt. CRM-197 was produced by Eurogentec for bioconjugation purposes. The heterobifunctional crosslinking reagent GMBS was used to conjugate Aβ1-6 peptide to CRM197. Reaction of CRM197 with an excess of GMBS reagent at room temperature followed by removal of GMBS and associated by-products by ultrafiltration/diafiltration. Excess peptide was then added to the GMBS activated CRM197 and allowed to react for 2 hours. Final product was purified by ultrafiltration and characterized. An equivalent amount of 1 μg of Aβ1-6 peptide was injected per mouse when the Aβ1-6 CRM vaccine was used. The amount of CRM conjugate used per mouse is 4.8 μg.

An equivalent amount of 1 μg of Aβ1-6 peptide was injected per mouse when the Aβ1-6 CRM197 peptide was used. The amount of CRM197 conjugate used per mouse was 4.8 μg. An equivalent of 5 μg of QS21 molecule per mouse was used when QS21 was injected. For the DOPC-cholesterol liposome formulation, 100 μg of DOPC and 25 μg cholesterol were injected together per mouse. Intramuscular injection of liposomal 3DMPL (5 ug of SUV MPL) or intramuscular injection 3DMPL itself at the 5 ug dose was used herein.

### Adjuvant Composition:

For AS01B, AS03 and AS15, the mouse dose is equal to 1/10^{th} of a human dose.
AS01B is composed of liposomes containing 3D-MPL and QS21 The mouse dose of AS01B contains 5 ug of 3DMPL co-formulated in neutral liposome, 5 ug of QS21. Those doses are per mouse and were injected using the intramuscular route (i.m.) 25 ul per mouse of AS01B+25 μl of PBS (phosphate buffer saline), or 25 μl of the appropriate peptide.
   Monocyte Analysis and Counting after Adjuvant Injection in Mice

24 hours after injection of each immunomodulator, peripheral blood was drawn from C57BL/6 mice via cardiac puncture with lithium-heparin as anticoagulant. Red blood cell lysis was performed twice on pooled blood with Ammonium Chloride-based Buffer (Sigma, Steinheim, Germany) and cells were counted with the EasyCount™ System (Immunicon). After one washing step, 500,000 cells were incubated with Rat anti-Mouse CD16/CD32 (BD Fc Block™ by BD Biosciences) for 10 min on ice and cells were further incubated for 30 min with a combination of the following directly conjugated antibodies at their pre-determined optimal concentration as described by Mildner et al., 2007: PerCP labeled-Streptavidin, PE-Hamster anti-Mouse CD3, Rat anti-Mouse CD45R/B220, Rat anti-Mouse Ly-6G, Mouse anti-Mouse NK1.1 APC-conjugated Rat anti-Mouse CD11 b, PE-Cy7-conjugated Hamster anti-Mouse CD11c, FITC-Rat Anti-Mouse Ly-6C (all from BD Biosciences) and Pacific Blue™ Rat anti-Mouse CD62L (BioLegend, San Diego, Calif.). Cells were finally washed three times and fixed for 15 min with a 2% paraformaldehyde solution in PBS. Fluorescence minus one (FMO) controls were always included in the assays for fluorescent compensation setting. Samples were acquired on a flow cytometer (BD FACSCanto II) and data analyzed with the FACSDiva software (BD Biosciences). Monocytes were identified by their Side/Forward scatter properties and gated as CD3-/CD45R/B220-/Ly-6G-/NK1.1-(Lineage-)/CD11b+ cells. CD11b+ monocytes frequency was reported as a percentage of the total cells excluding debris.

### Ex Vivo Uptake Assay of Aβ42

Preparation of cells: Peripheral blood was drawn from C57BL/6 mice via cardiac puncture with lithium-heparin as anticoagulant, 24-Hour after injection of the adjuvants used herein. Red blood cell lysis was performed twice on pooled blood with Ammonium Chloride-based Buffer (Sigma, Steinheim, Germany) and cells were counted with the EasyCount™ System (Immunicon).

Cell stimulation/Aβ phagocytosis: cells were seeded at 10⁶ cells/mL onto a 24-well tissue culture plate and stimulated for 2 or 24 h in the presence or absence of 1 μg/ml of Aβ1-42 HiLyte Fluor™488 (Anaspec, Fremont, Calif.), which was pre-incubated or not for 1 h with 1 μg/ml of anti-amyloid β antibodies (e.g. polyclonal mouse antibody raised against Aβ1-6CRM197 or a mouse monoclonal antibody specific to Ab1-7: both antibodies developed by GSK).

FACS analysis: cells were harvested after incubation with fluorescent Aβ peptide with Trypsin/EDTA and cold PBS and washed three times. 500,000 cells were incubated in 96-well plate for 10 min. on ice in the presence of Rat anti-Mouse CD16/CD32 (clone 2.4G2—BD Fc Block™) and further stained for 30 min. with a combination of the following directly conjugated antibodies at their pre-determined optimal concentration: PE-Hamster anti-Mouse CD3 (clone 145-2C11), Rat anti-Mouse CD45R/B220 (clone RA3-6B2), Rat anti-Mouse Ly-6G (clone 1A8), Mouse anti-Mouse NK1.1 (clone PK136), APC-conjugated Rat anti-Mouse CD11 b (clone M1/70), PE-Cy7-conjugated Hamster anti-Mouse CD11c (clone HL3), (all from BD PharMingen). Cells were finally washed twice and fixed for 15 min. with a 2% paraformaldehyde solution in PBS. FMOs controls were always included in the assays.

Samples were acquired on a flow cytometer (BD FACSCanto II) and data analyzed with the FACSDiva software (BD Biosciences).

Monocytes were identified by their Side/Forward scatter properties, excluding debris and gated as CD3-/CD45R/B220-/Ly-6G-/NK1.1-(Lineage-)/CD11b+ cells. Aβ uptake was assessed by reporting the percentage and Mean Fluorescence Intensity (GeoMean) of positive HiLyte fluor488 Aβ1-42 cells among gated monocytes.

### Quantification of Anti-Amyloid Beta 1-42 Antibodies in Mice Serum Using ELISAs

Whole blood was collected from mice and centrifuged on a vacutainer blood collection tube containing gel for serum separation. Serum samples were stored at −80° C. Streptavidin-coated plates (Greiner Bio-One, Germany) were first coated with either beta-amyloid (1-42)-Lys(Biotin)-NH2 peptide (Anaspec, Inc.) at 0.5 μg/mL, using 50 mM sodium carbonate buffer, overnight at 4° C. Plates were then washed using a 4 times using PBS/0.05% Tween 20. Super Block (ScyTek laboratories) was added to the plates and incubated at 37° C. for at least one hour. Serum samples and standard (anti Aβ42 antibody (6E10 antibody, Covance, Inc.) were serially diluted in the plates and incubated at 37° C. for 2 hours. After a wash step, diluted peroxidase Affini Pure goat anti-mouse IgG, Fcγ fragment specific (Jackson ImmunoResearch Laboratories Inc.) was added for 1 hour at 37° C. A last wash was performed before adding TMB substrate reagent (BD OptEIA™, BD Biosciences) for 30 min at room temperature (RT). Immediately, plates were stopped using 2N sulfuric acid, and then read at 450 nm using SpectraMax microplate reader (Molecular Devices, Inc.). Data analysis using the OD half max method was performed using the Soft Max Pro (Molecular Devices Inc) and GraphPad prism (GraphPad Inc) softwares. Statistical analysis was done using SAS and Unistat platform. Results of the multiple comparison analysis are shown in the following table.

| | | | Significant? |
|---|---|---|---|
| Tukey's Multiple Comparison Test | Mean Diff. | q | P < 0.01? |
| Aβ1-6-CRM197 + PBS vs Aβ1-6-CRM197 + QS21 | −58490 | 5.096 | Yes |
| Aβ1-6-CRM197 + PBS vs Aβ1-6-CRM197 + | −170500 | 14.86 | Yes |
| QS21 + Liposome | | | |
| Aβ1-6-CRM197 + PBS vs Aβ1-6-CRM197 + Liposomes | 82.44 | 0.007183 | No |
| Aβ1-6-CRM197 + QS21 vs Aβ1-6-CRM197 + | −112000 | 9.762 | Yes |
| QS21 + Liposome | | | |
| Aβ1-6-CRM197 + QS21 vs Aβ1-6-CRM197 + Liposomes | 58570 | 5.103 | Yes |
| Aβ1-6-CRM197 + QS21 + Liposome vs Aβ1-6-CRM197 + | 170600 | 14.87 | Yes |
| Liposomes | | | |

### Results

As shown in FIG. 20, flow cytometry analysis demonstrated that the intramuscular injection of DQ (QS21+liposome) trigger the monocytes to be able to uptake an higher amount of 442 compared to PBS or QS21 (5 ug) injected mice or intramuscular injection of liposomal MPL (5 ug of SUV MPL) or intramuscular injection MPL itself at the 5 ug dose used herein.

To better compare the liposomal formulation of QS21 compared to the single component separately in animals, we performed innate immunity analysis by counting the monocytes in the peripheral blood from injected mice. A single injection of QS21+liposome promotes after 24 hrs an increase in the number of monocytes that could reach up to 13% of total peripheral white blood cells (FIG. 19A) compared to QS21 alone group (9.1%). Moreover, we observed an increased number of activated Ly6C+ monocytes (86%) after QS21+liposome treatment compared to PBS (69%) or QS21 alone (75%) that were stained by flow cytometry using the monocyte staining cocktails previously described to be an efficient method to identify monocytes that give rise to CNS macrophages or microglia and to measure their activation state by using the Ly6C marker, which has been shown to be the main marker to follow monocyte activation (Mildner A et al. Nat Neurosci. 2007 December; 10(12):1544-53). To measure whether those monocytes were able to clear Aβ amyloid, we performed an 442 uptake assay and observed that QS21+liposome injection was promoting the 442 uptake by peripheral blood monocytes (FIG. 20). In order to measure the phagocytic activity, we have used fluorescent HiLyteFluo 442 peptides (Anaspec Inc). Flow cytometry analysis demonstrated that intramuscular injection of DQ (QS21+liposome) triggers the monocytes to uptake an higher amount (2 fold) of 442 compared to PBS or QS21 (5 μg) injected mice (FIG. 20). This suggests that QS21 alone does not promote Aβ uptake and the QS21 combined with liposome is necessary to promote this effect.

Furthermore, the liposomal QS21 formulation was assessed for its ability to enhance the humoral immune response. Four groups of mice received the following treatments: Aβ1-6 CRM; Aβ1-6 CRM+QS21; Aβ1-6 CRM+liposomes; and Aβ1-6 CRM+QS21+liposomes (FIG. 21). The liposomal QS21 formulation and its separate components were combined with an Aβ antigen (native Aβ1-6 conjugated to CRM197) to analyse their effect on antibody response. Immunization at day 0-14 and 21 was performed and sera were collected at day 28, and the anti-Aβ1-42 IgG specific titers measured. Statistical analysis using ANOVA and Tukey's multiple comparison tests show that the liposome+QS21+Aβ1-6 CRM197 provided a significantly higher anti-Aβ1-42 antibody titre than QS21+Aβ1-6 CRM197. QS21+Aβ1-6 CRM197 group is not different to the liposome+Aβ1-6 CRM197 group, suggesting that the combination of liposome and QS21 provides an improved immune response toward Aβ42 compared to QS21 alone.

### Example 20

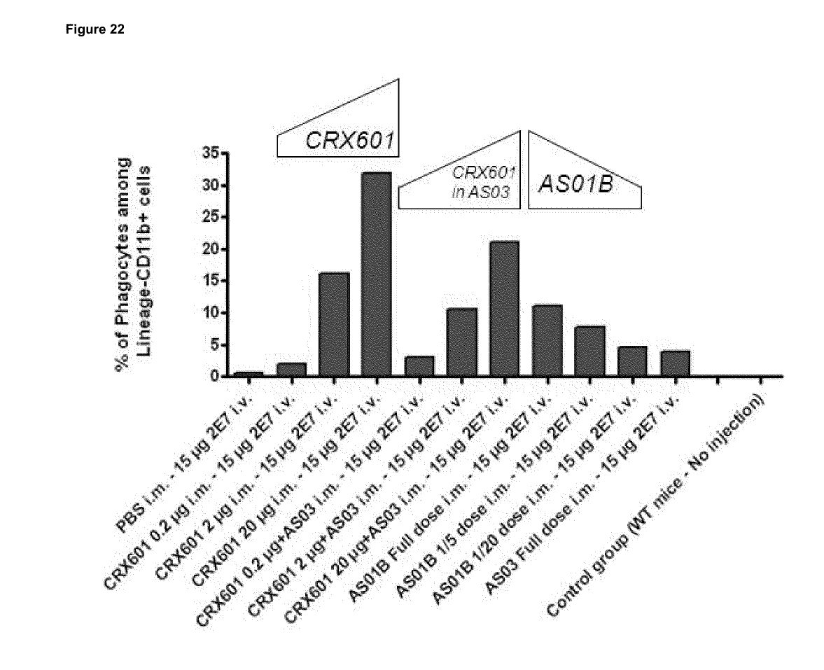

### FIG. 22

Dose-range of AS01B or CRX601 for antibody-mediated Aβ peripheral uptake after a single injection (24 hrs time point)

Rapid Aβ uptake in mouse peripheral blood monocytes in single intra muscular injected animals with different doses of AS01B or CRX601 with or without AS03 emulsion after intra vascular injection of constant dose of anti Aβ1-7 monoclonal antibody and ectopic Aβ Fluor. Results show that TLR4 containing adjuvant triggers a higher antibody-mediated Aβ uptake. Dose-response observed from AS01B and CRX601+ or −AS03 adjuvants. CRX601 at 20 ug triggers highest Aβ uptake and the effect is partially quenched when combined with AS03 emulsion.

In Vivo Uptake Assay of Aβ42 after Immunization or Passive Immunotherapy:

Mice: C57BL/6 female mice were purchased from Charles River.

Mice were passively immunized with a constant dose of anti Aβ monoclonal antibody (15 ug of 2E7)+different dose of immunomodulators (CRX601 or CRX601/AS03 or AS01B). 24 hr later, 5 μg per mouse of fluorescent HiLyte Fluor™ 488-labeled Amyloid γ1-42 (Anaspec, Fremont, Calif.) was injected in the tail vein. 2 hrs later, peripheral blood was drawn from immunized C57BL/6 mice via cardiac puncture with lithium-heparin as anticoagulant, Plasma of pooled blood was decanted and saved for measurement of antibody titres by ELISA. Red blood cell lysis was performed twice with Ammonium Chloride-based Buffer (Sigma, Steinheim, Germany) and cells were counted with the EasyCount™ System (Immunicon).

FACS Analysis: 500,000 cells were aliquoted in a 96-well plate, washed once, incubated for 10 min. on ice in the presence of Rat anti-Mouse CD16/CD32 (clone 2.4G2-BD Fc Block™) and further stained for 30 min. with a combination of the following directly conjugated antibodies at their pre-determined optimal concentration: PE-Hamster anti-Mouse CD3 (clone 145-2C11), Rat anti-Mouse CD45R/B220 (clone RA3-6B2), Rat anti-Mouse Ly-6G (clone 1A8), Mouse anti-Mouse NK1.1 (clone PK136), APC-conjugated Rat anti-Mouse CD11b (clone M1/70), PE-Cy7-conjugated Hamster anti-Mouse CD11c (clone HL3), (all from BD PharMingen). Cells were finally washed twice and fixed for 15 min. with a 2% paraformaldehyde solution in PBS. FMOs controls were always included in the assays.

Samples were acquired on a flow cytometer (BD FACSCanto II) and data analyzed with the FACSDiva software (BD Biosciences).

Monocytes were identified by their Side/Forward scatter properties, excluding debris and gated as CD3-/CD45R/B220-/Ly-6G-/NK1.1-(Lineage-)/CD11b+ cells. Aβ uptake was assessed by reporting the percentage and Mean Fluorescence Intensity (GeoMean) of positive HiLyte Fluor™ 488 Aβ1-42 cells among gated monocytes.

### Example 21

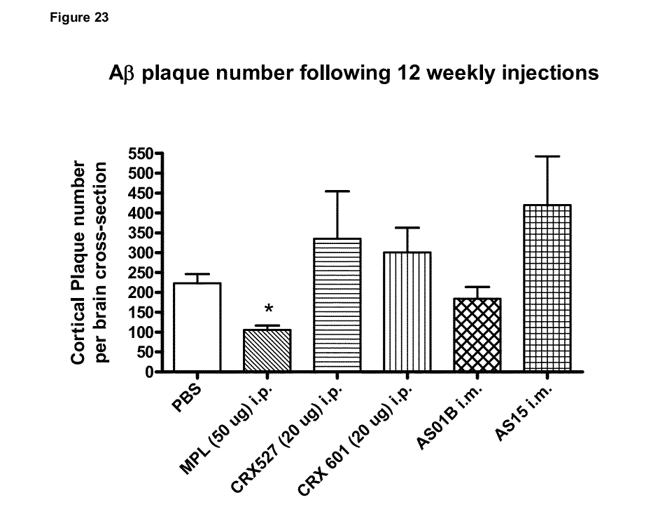

### FIGS. 23 to 26

To determine whether TLR4 agonist will improve the cognitive impairment and clearance of Aβ in APP/PS1 mice.

### Injections

Five groups of APP_{Swe}/PS1 mice received one a week for a period of 12 weeks the following treatment:
Gr1: APP_{Swe}/PS1+NaCl 0.9% n(=20), i.p. injections
Gr4: APP_{Swe}/PS1+CRX527 (20 μg/mouse, 130 μl, n=10), i.p. injections
Gr5: APP_{Swe}/PS1+CRX601 (20 μg/mouse, 130 μl, n=10), i.p. injections
Gr6: APP_{Swe}/PS1+3D MPL (50 μg/mouse, 130 μl, n=10), i.p. injections
Gr8 APP_{Swe}/PS1+AS15 ( 1/10th of human dose, 50 μl, n=10), i.m. injections
Gr9: APP_{Swe}/PS1+AS01B ( 1/10th of human dose, 50 μl, n=10), i.m. injections

| Group | Agonist | n = t₀ | n = t₁₂ |
|---|---|---|---|
| 1 | Saline | 20 | 19 |
| 4 | CRX527 | 10 | 9 |
| 5 | CRX601 | 10 | 10 |
| 6 | 3D-MPL | 10 | 9 |
| 8 | AS15 | 10 | 10 |
| 9 | AS01B | 10 | 10 |

### Behavioral Analyses

### T-Water Maze

Mice were tested during the “light on” phase of the day. Behavioral experimenter was blinded to the genetic and treatment status of animals. To assess hippocampal-dependent spatial learning and memory, mice were trained in the T-water maze task. In this paradigm, we evaluate the mouse's ability to remember the spatial location of submerged platform. The T-maze apparatus (length of stem, 64 cm; length of arms, 30 cm; width, 12 cm; height of walls, 16 cm) was made of clear fiberglass and filled with water (23±1° C.) at a height of 12 cm. A platform (11×11 cm) was placed at the end of the target arm and was submerged 1 cm below the surface. The acquisition phase allows to evaluate animals for left-right spatial learning. During the first two trials, platforms were placed on each arms of the maze to test the spontaneous turning preference of the mouse. After these two trials, the least chosen arm was reinforced by the escape platform. The mice were placed in the stem of the T-maze and choose to swim either left or right until they found the submerged platform and escape to it, to a maximum of 60 s. After reaching the platform, the mice remained on it for 20 s and then were immediately placed back in the maze. If the animals did not find the platform within this limit, they were gently guided onto it. Repeated trials were presented on the same day up to a maximum of 48 trials. A rest period of at least 10-15 min intervened between each block of 10 trials. A mouse was considered to have learned the task when it made no errors in a block of five consecutive trials. The reversal learning phase was then conducted 48 h later. During this phase, the same protocol was repeated, except that the mice were trained to find the escape platform on the opposite side to that on which they had learned on acquisition phase. The number of trials to reach the criterion (five of five correct choices made on consecutive trials) was measured as well as the latency to find the escape platform.

### Passive Avoidance Test

Based on the animal's natural tendency to prefer the dark environment, the animals were also evaluated in retention of non-spatial memory for one-trial passive avoidance task. The passive avoidance apparatus (Ugo Basile) was divided into two sections, one illuminated (the start compartment) and one dark (escape compartment). The floor of each compartment contained a grid, with only the dark compartment being electrified by a generator. On the training day, mice were placed into the lighted compartment for 60 s acclimation period. The guillotine door was then opened, and the latency to enter the dark side was recorded. Immediately after entering the dark compartment, the door was closed and an electric shock (0.5 mA for 2 s) was delivered. The mouse was kept in the dark compartment for 10 s before being returned to its home cage. On the next day, the mice were again placed in the light compartment, and the time, step through latency to enter the dark side, was measured for up to 300 s.

### Nesting Behaviour

Thereafter, the nesting behaviour was used to test for changes in emotional status (e.g. apathy). Reduced nesting has been observed in hippocampal lesioned mice and mouse models of Alzheimer's disease (Deacon R M. Assessing nest building in mice. Nat Protoc. 2006; 1(3):1117-9. PubMed PMID: 17406392). Animals were individually housed in a cage containing sawdust and in which a 5×5 cm piece of cotton was introduced to allow nesting behaviour. One day later, the quality of the nest was determined according to a five-point scale as described by Deacon (2006): 1—Nestlet apparently untouched, 2—Nestlet partially torn up, 3—Nestlet mainly shredded but no apparent presence of nesting site, 4—Observable flat nest, 5—Observable (near) perfect nest.

### Tissue Analyses

Mice were anesthetized under isoflorane and blood was drawn via cardiac puncture before head decapitation. Brains were rapidly removed from the skulls and placed in cold phosphate buffered saline (PBS) solution. Then hemibrains were separated and olfactory bulbs and cerebellum were removed. One hemibrain was rapidly frozen in liquid nitrogen and stored at −80° C. for protein analysis. The other one was postfixed for 2-4 days in 4% paraformaldehyde (PFA), pH 9.5 at 4° C., and then placed in a PFA solution containing 10% sucrose overnight at 4° C. The frozen brains were mounted on a microtome (Reichert-Jung) and cut into 25-μm coronal sections. The slices were collected in cold cryoprotectant solution (0.05 M sodium phosphate buffer, pH 7.3, 30% ethylene glycol, and 20% glycerol) and stored at −20° C. until immunocytochemistry or in situ hybridization histochemistry.

### Stereological Analysis.

An observer who was blind to the treatment status of the material did all quantitative histological analyses. To count Aβplaques, sections of APP_{Swe}/PS1 mice were immunostained for Aβ (polyclonal mouse anti-Aβ 6E10, 1:3000; Covariance) as previously reported (Richard et al., 2008; Simard et al., 2006). Two sections were chosen for prefrontal cortex at +2.34 and +2.10 mm from the bregma according to a stereotaxic atlas (Paxinos and Franklin, second edition) and four sections for hippocampus/cerebral cortex at −1.70, −1.94, −2.46 and −2.92 mm. Unbiased stereological analysis was performed as described previously (Boissonneault et al., 2009; Richard et al., 2008; Simard et al., 2006). Briefly, the contours of the prefrontal cortex, the hippocampus and the cortex areas were traced as virtual overlay on the steamed images and areas were calculated. The area occupied by all AB-labeled plaques was determined. Real-time images (1600_{—}1200 pixels) were obtained using a Nikon C80i microscope equipped with both a motorized stage (Ludl) and a MicrofireCCD color camera (Optronics). Such an apparatus was operated using the Stereo Investigator software designed by Microbrightfield. Both cortex and hippocampus areas were traced using a Cintiq 18S interactive pen display (Wacom).

### Protein Extraction and Detection of Total Aβ Levels by Western Blot.

Proteins from hemi-forebrains were extracted using a modified method of the procedure published by Lesné et al (Lesné et al., 2006). All manipulations were done on ice to minimize protein degradation. One hemi-forebrain was placed in a 1 ml syringe with a 20 G needle. 500 μl of buffer A (50 mM Tris-HCl pH 7.6, 0.01% NP-40, 150 mM NaCl, 2 mM EDTA, 0.1% SDS, 1 mM phenylmethylsulfonyl fluoride (PMSF), protease inhibitor cocktail) were added and 10 up and down were made to homogenize the tissue, followed by a 5 minutes centrifugation at 3 000 RPM at 4° C. The supernatant (extracellular proteins enriched fraction) was then collected and frozen at −80° C. The insoluble pellet was suspended in 500 μl TNT-buffer (Buffer B; 50 mM Tris-HCl pH 7.6, 150 mM NaCl, 0.1% Triton X-100, 1 mM PMSF, protease inhibitor cocktail), followed by a 90 minutes centrifugation at 13 000 RPM at 4° C. The supernatant (cytoplasmic proteins enriched fraction) was then collected and frozen at −80° C. The pellet was suspended in 500 μl buffer C (50 mM Tris-HCl pH 7.4, 150 mM NaCl, 0.5% Triton X-100, 1 mM EGTA, 3% SDS, 1% deoxycholate, 1 mM PMSF, protease inhibitor cocktail) and incubated at 4° C., 50 RPM, for 1 hour. The samples were centrifuged for 90 minutes at 13 000 RPM and 4° C. and the supernatant (membrane proteins enriched fraction) was collected and frozen at −80° C. Protein concentration of each fraction was determined using the Quantipro BCA assay kit (Sigma) according to the manufacturer protocol.

For total Aβ detection, 10-30 μg of extracellular, cytoplasmic and membrane protein fractions were separated on a precast 10-20% SDS polyacrylamide Tris-Tricine gel (Bio-Rad). Separated proteins were then transferred onto polyvinylidene fluoride (PVDF) membranes (PerkinElmer Life and Analytical Sciences) and detected by Western blotting. Blots were probed with a mouse anti-amyloid beta protein monoclonal antibody clone 6E10 (1:1000, Covariance) in 1 M Tris-HCl, pH 8.0, 5 M NaCl, 5% skim milk, and 0.05% Tween 20. Blots were visualized using anti-mouse secondary antibody tagged with horseradish peroxidase (1:1000; Jackson Immuno-Research) and enhanced chemiluminescence (PerkinElmer Life and Analytical Sciences). Membranes were stripped in 25 mM glycine-HCl, pH 2.0, containing 1% SDS to allow actin revelation using first a mouse actin antibody (MAB1501, 1:5000; Millipore Bioscience Research Reagents) and then a goat anti-mouse peroxidase conjugated secondary antibody (1:1000; Jackson ImmunoResearch).

Quantification was done by determining integrative density of the bands using a gel imaging system (scanner Agfa Arcus II; NIHImage J software version 1.32j) and background values were removed. Optical values were normalized according to the actin loading control. Results are expressed as mean±SEM.

### Results

Results for Aβ total plaque loading are shown in FIG. 23. Aβ total plaque loading analyses reveal a significant difference between the PBS control group versus the 3D MPL group in term of Abeta plaque loading measurement by immunofluorescence. An asterisk (*) indicates ANOVA, P=0.05 vs PBS group.

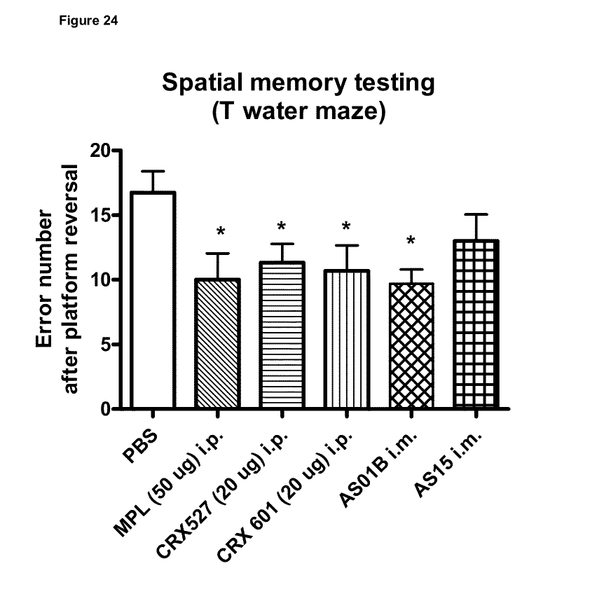

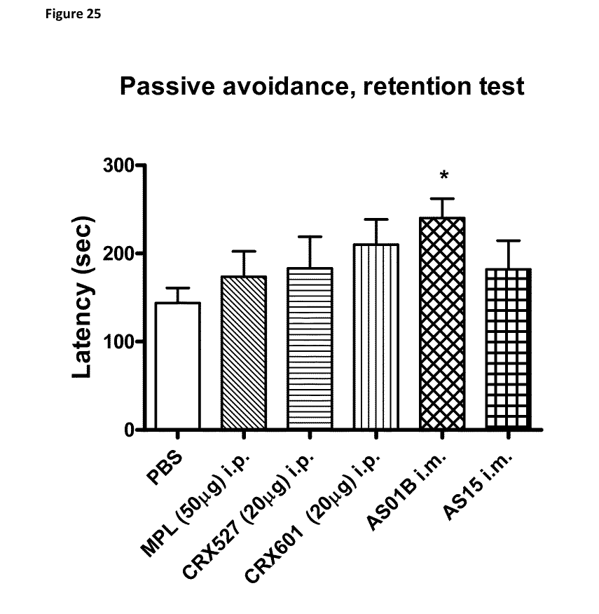

Results for behavioural analysis are provided in FIGS. 24 and 25.

In FIG. 24 Twelve weekly injections of 3D MPL or CRX527 or CRX601 or AS01B in APP/PS1 mouse model shows a spatial memory improvement compared to non treated mice

In FIG. 25 AS01B treated animals exhibit a significant retention score compared to non-treated animals. Step through latencies were measured during the passive avoidance after the 12^{th} weekly injection. Data are expressed as mean (+/−SEM (One-way ANOVA)).

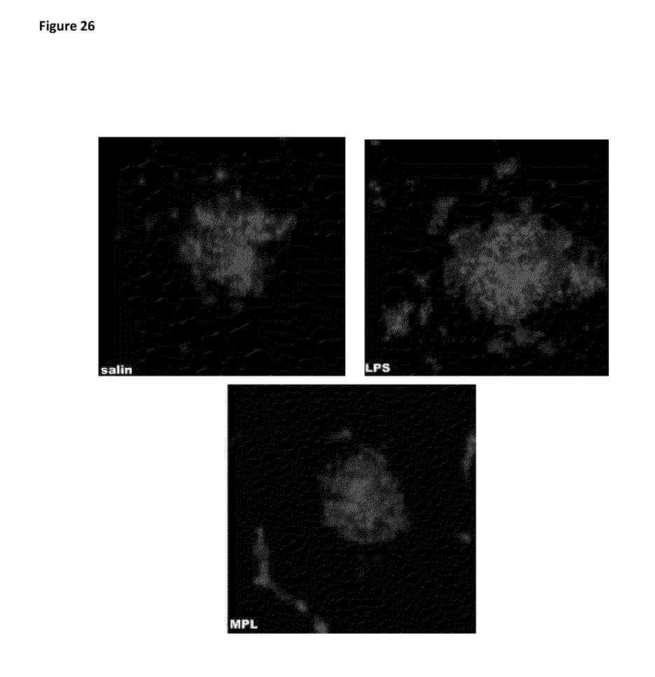

Representative results for brain histology are provided in FIG. 26. Representative immunofluorescence picture of amyloid plaques stained using the anti Aβ antibody (clone 6E10, protocol as described by Covance Inc, USA) counted are shown in FIG. 23. Brain from mouse treated with MPL showed more compacted plaques with less total area. In contrast, the plaques from LPS treated group showed large diffuse amyloid plaques (FIG. 26).

3D-MPL provides a statistically significant reduction in Amyloid beta plaque number.

3D-MPL, CRX 527, CRX 601 and AS01B all provide significant improvements in behaviour as assessed by T water maze testing.

### Example 22

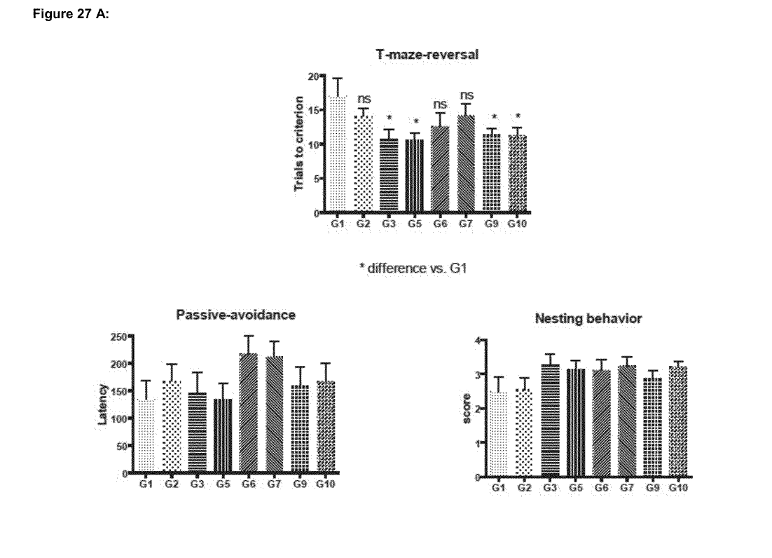

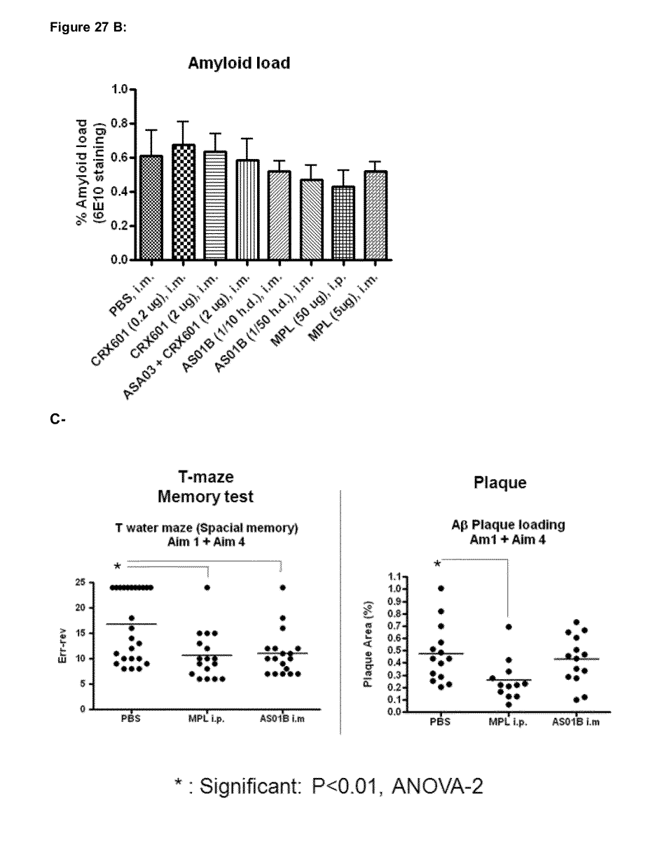

### FIG. 27

A second prophylactic experiment in APP/PS1 mouse model (Jackson's laboratory Inc) was performed to further investigate four objectives:
1. Evaluate MPL in two type of route injection (intra muscular and intra peritoneal)
2. Evaluate AS01B in two physiological doses for the intra muscular route
3. Evaluate others synthetic TLR4 agonist such as CRX-601 in 2 doses
4. Investigate for the first time CRX-601 co-formulated in the AS03 emulsion

Therefore, the following adjuvants or immunomodulators were used in the following groups:

Gr 1: PBS i.m.—12×weekly, Negative control [n=10 (2 females]
Gr 2: CRX-601 i.m. (0.2 ug per mouse) [n=10 (2 females)]
Gr 3: CRX-601 i.m. (2 ug per mouse), 12×weekly [n=10 (2 females)]
Gr 5: AS03-CRX601 (2 ug dose for CRX601, 1/10 human dose for AS03, i.m. 12×weekly [n=10 (2 females)]
Gr 6: AS01B i.m ( 1/10 human dose), 12×weekly [n=10 (2 females)]
Gr 7: AS01B i.m ( 1/50 human dose), 12×weekly [n=10 (2 females)]
Gr 9: 3D 3D MPL intra peritoneal (as it in Aim 1), 50 ug per mouse, 12×weekly [n=10 (2 females)]
Gr 10: 3D MPL i.m. (5 ug per mouse), 12×weekly [n=10 (2 females)]

Results indicate that groups 3, 5, 9 and 10 were significantly improved in the T maze reversal test compared with group 1.

These last described formulations were administered once a week in 3 months-old APP_{swe}/PS1 mice for 12 consecutive weeks. Cognitive functions and Aβ deposition were assessed for each APP_{swe}/PS1 mouse (FIG. 27A). We evaluated the hippocampus-based spatial learning and memory in the T-water maze behavioral test. The group 3 (CRX601 at 2 μg), group 5 (CRX601 in AS03), group 9 (3D MPL using the intra peritoneal route) and group 10 (3D MPL using the intra muscular route) showed significant improvement of their cognitive functions in comparison to the PBS-treated control group using the statistical unpaired t-tests. Although, AS01B treatment at 1/10 human dose seemed to slightly ameliorate the cognitive performances of APP_{swe}/PS1 mice but the difference was not statistically significant. The passive avoidance and nesting behaviour tests were not able to show significant improvement despite few groups such as the group 6 (AS01B; 1/10 human dose) and group 7 (AS01B; 1/50 human dose) showed a trend of improvement. Since cerebral Aβ accumulation is suggested to be the cause of these cognitive deficits, the level of amyloid plaques in the brain was quantified. Although, not completely statistically significant because of the low number of mice in that experiment, we observed once again a trend of reducing the Aβ amyloid plaque after the administration of MPL using the intra peritoneal route (FIG. 27B). To statistically powered our analysis using ANOVA-2 tests, we combined the data from the two replicated experiments described herein, i.e., Example 21 and 22 experiments. As shown in FIG. 27C, the only 2 groups that showed statistical significance using ANOVA-2 tests were the MPL intra peritoneal and the AS01B group in the T-water tests. For plaque loading, only the MPL intra peritoneal group showed a statistical significant plaque loading reduction (P<0.01) within a 95% confidence interval.

### Example 23

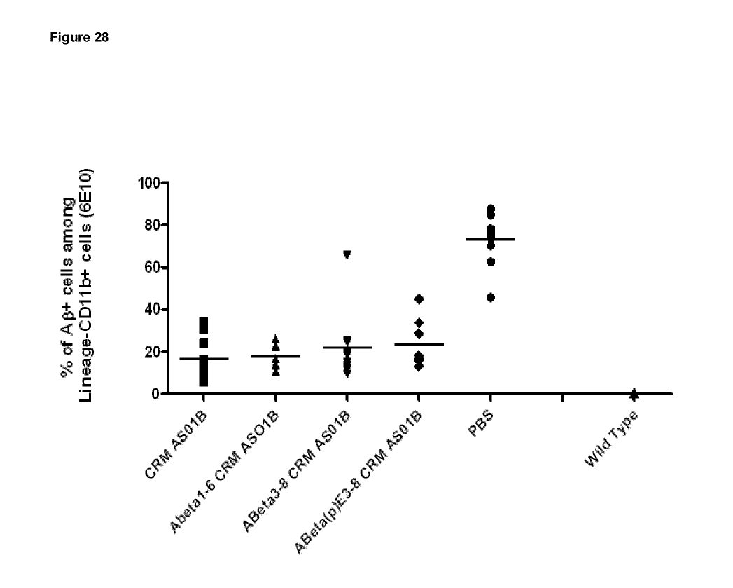

### FIG. 28

To measure whether AS01B CRM (CRM197 used herein) with or without conjugated peptides affect the endogenous Aβ level in the peripheral blood of TASTPM amyloid accumulating mouse model (Howlett D R, Richardson J C, Austin A, Parsons A A, Bate S T, Davies D C, Gonzalez M I. Cognitive correlates of Abeta deposition in male and female mice bearing amyloid precursor protein and presenilin-1 mutant transgenes. Brain Res. 2004; 1017(1-2):130-6. PubMed PMID: 15261108).

A schedule of immunisation over one year was used and was considered a prophylactic model because we started the treatment before the Aβ accumulation and extend that treatment until the mice were 1 year old. Mice were immunized ten times starting at 3 months old. 25 mice per group were used at starting time for statistical reason based on power calculation from the previous experiment and from the fact that we could lose 10% of the mice because of high incidence of natural death in that transgenic model. Intramuscular injections starting at 3 months were called for logical reason: day 0, day 28, 56, 84, 112, 140, 168, 196, 224 and day 252. The amyloid accumulating mouse model (TASTPM) was used herein. A few untreated mice were used herein as controls. Five immunization groups (25 mice per immunization group) in TASTPM
1—CRM+AS01B
2—Aβ1-6 CRM+AS01B
3—Aβ3-8CRM+AS01B
4—Aβ(p)E-8CRM+AS01B
5—PBS

The Aβ fragments conjugated to CRM were used at 1 ug per mouse based on peptide content. At day 253, peripheral blood was drawn from mice via cardiac puncture and monocytes were analyzed by flow cytometry to measure the amount left of amyloid β in the monocytes after that long prophylactic treatment. The results showed that most of the AS01B containing groups do exhibit less Aβ in the peripheral blood cells such as the monocytes (CD11b+, Lin− and Aβ+) compared to the PBS treated group which showed higher amount of Aβ in the peripheral monocytes. This result suggests that AS01B with or without antigen co-formulated promotes the Aβ clearance in the long term since the Aβ level is low in the AS01B treated group compared to the placebo control (PBS).

### Method:

Mice were immunized ten times (at Day 0, 28, 56, 84, 112, 140, 168, 196, 224 and 252) intramuscularly (1 ug per mouse based on peptide content) At day 56, peripheral blood was drawn from immunized mice via cardiac puncture with lithium-heparin as anticoagulant. Plasma of individual mice were decanted and saved for measurement of antibody titres by ELISA. Red blood cell lysis was performed twice with Ammonium Chloride-based Buffer (Sigma, Steinheim, Germany) and cells were counted with the EasyCount™ System (Immunicon).

FACS analysis: 500,000 cells were aliquoted in a 96-well plate, washed once, incubated for 10 min. on ice in the presence of Rat anti-Mouse CD16/CD32 (clone 2.4G2—BD Fc Block™) and further stained for 30 min. with a combination of the following directly conjugated antibodies at their pre-determined optimal concentration: PE-Hamster anti-Mouse CD3 (clone 145-2C11), Rat anti-Mouse CD45R/B220 (clone RA3-6B2), Rat anti-Mouse Ly-6G (clone 1A8), Mouse anti-Mouse NK1.1 (clone PK136), APC-conjugated Rat anti-Mouse CD11 b (clone M1/70), PE-Cy7-conjugated Hamster anti-Mouse CD11c (clone HL3), (all from BD PharMingen). Cells were finally washed twice and fixed for 15 min. with a 2% paraformaldehyde solution in PBS. FMOs controls were always included in the assays.

Samples were acquired on a flow cytometer (BD FACSCanto II) and data analyzed with the FACSDiva software (BD Biosciences).

Monocytes were identified by their Side/Forward scatter properties, excluding debris and gated as CD3-/CD45R/B220-/Ly-6G-/NK1.1-(Lineage-)/CD11b+ cells. Aβ uptake was assessed by reporting the percentage and Mean Fluorescence Intensity (GeoMean) of positive HiLyte Fluor™ 488 Aβ1-42 cells among gated monocytes.

### Ref:

Howlett D R, Richardson J C, Austin A, Parsons A A, Bate S T, Davies D C, Gonzalez M I. Cognitive correlates of Abeta deposition in male and female mice bearing amyloid precursor protein and presenilin-1 mutant transgenes. Brain Res. 2004 Aug. 13; 1017(1-2):130-6. PubMed PMID: 15261108.

### Example 24

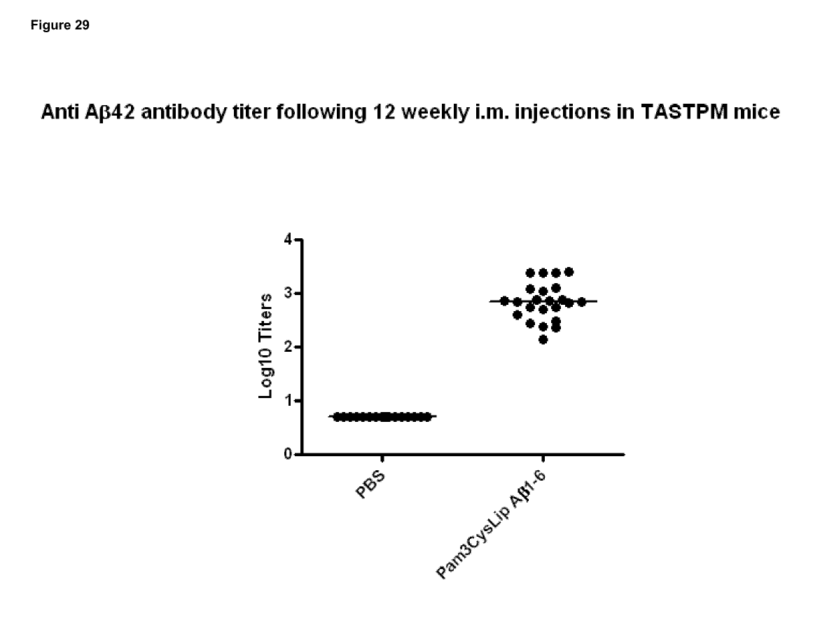

### FIG. 29

High Aβ42 specific Immunogenicity promoted by the TLR2 containing adjuvant Pam3CysLip peptide fused with an amyloid the Aβ fragment model (Aβ1-6). The sequence of the TLR2 agonist used herein (Pam3CysLip Aβ1-6) is: Pam3Cys—SQ EPAAPAAEAT PAAEAPDAEFRH (SEQ ID NO:9).

High and specific anti Aβ42 specific immunogenicity is promoted by a TLR2 containing adjuvant fused to the first six amino acid of Aβ in the amyloid deposition mouse model (TASTPM) following 12 injections of 10 ug of the Pam3Cys—SQ EPAAPAAEAT PAAEAP DAEFRH (SEQ ID NO:9) using the intramuscular route.

### Example 25

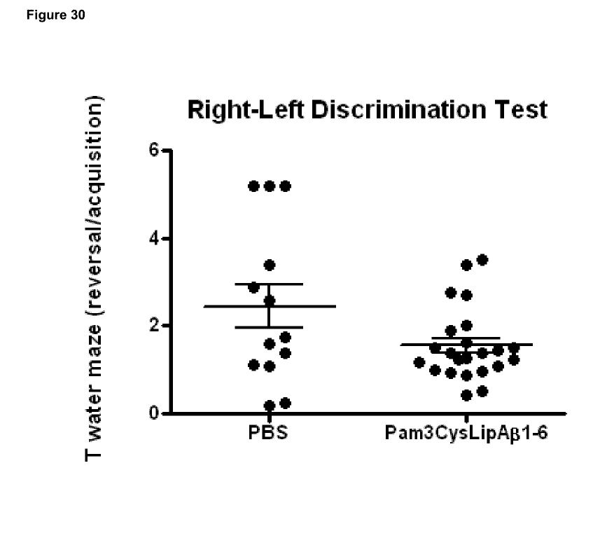

### FIG. 30

A TLR2 agonist containing formulation could improve the working memory in amyloid deposition model (TASTPM).

To determine whether a TLR2 agonist will improve the cognitive impairment in TASTPM mice, the Pam3CysLip adjuvant fused to the Aβ1-6 peptide was injected 12 times at weekly basis using the intramuscular route and the memory was assessed using the T-water maze test. The comparison with the PBS injected group shows that a trend of improvement in the right-left discrimination tests is observed after the 12 injections of Pam3CysLip Aβ1-6 peptide. TASTPM mice have received one a week for a period of 12 weeks the following treatment: PBS or mice have received once a week for a period of 12 weeks the following treatment: PBS or of Pam3CysLip Aβ1-6 peptide.

### Example 26

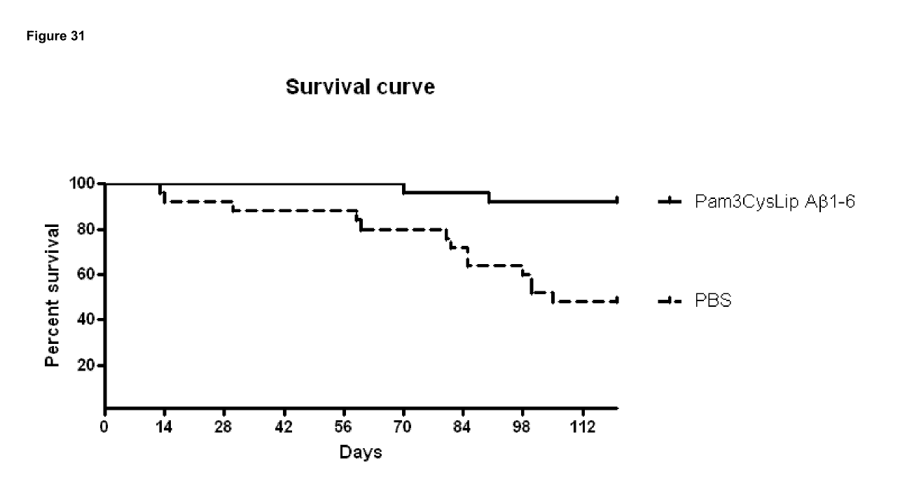

### FIG. 31

The usage of the TLR2 agonist, i.e. Pam3CysLip Aβ1-6 peptide, improves the survival rate of TASTPM mouse model. 23 out of 25 mice survived after weekly injections of Pam3CysLip Aβ1-6 peptide compared to PBS injected animals in which only 13 out 25 TASTPM mice survived (followed for 112 days). The Kaplan-Meier survival curve is shown in FIG. 31.

### Example 27

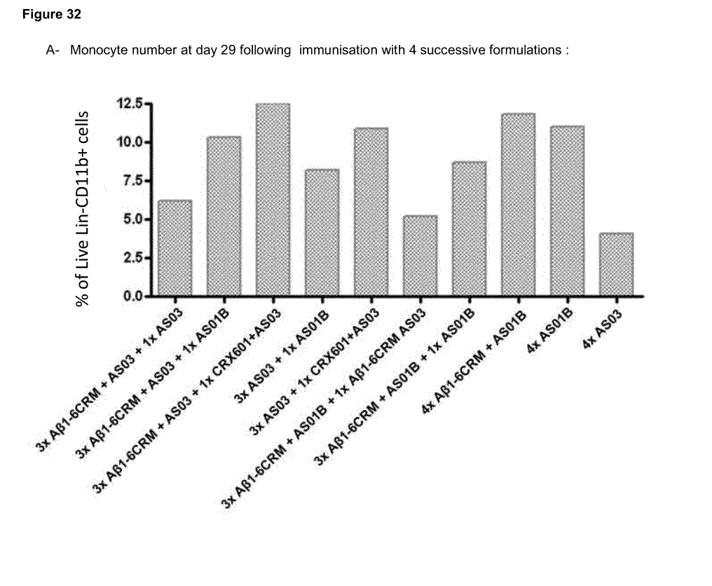

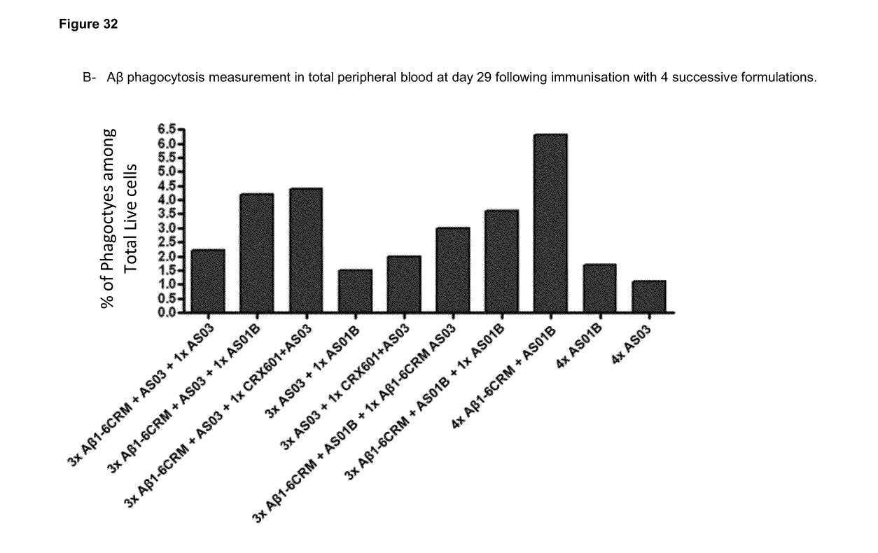

### FIG. 32

Evaluation of the impact of TLR4-containing adjuvants, i.e. AS01B and CRX601/AS03, on in vivo amyloid β1-42 uptake during and after vaccination inducing a polyclonal anti-Aβ1-42 antibody response.

These results (FIG. 32) demonstrate that TLR4-containing adjuvants AS01B and CRX601 in AS03 emulsion enhance the monocyte number (FIG. 32A). Moreover, we observe that the final boost at day 29 with TLR4-containing adjuvant promotes the Aβ uptake (FIG. 32B). In particular, this phenomenon of high amyloid uptake is higher in the groups that exhibit high antibody response as shown in FIG. 32C. The results show that 4 consecutive immunizations with AS01B+Aβ1-6 CRM induce the highest in vivo Aβ uptake. The results show that the stimulation of the innate immune system is the trigger for this enhanced antibody-mediated phagocytosis since the mice that were immunized with 3 consecutive injections of AS03+Aβ1-6 CRM and, then, a final boost of AS01B (TRL4-containing AS) alone (without antigen) before the phagocytic assay show a higher Aβ1-42 uptake compared to animals injected 3× with Aβ1-6 CRM followed with the boost of AS03 alone (without TRL4). Similarly, a higher Aβ1-42 uptake is observed in the groups immunized 3 times with AS03+Aβ1-6, then, CRX601/AS03 vs. AS03+Aβ1-6, then, AS03 alone, which confirms the importance of the TLR4 agonists in this formulation to activate the innate immune system leading to an increased phagocytosis. In contrast, the AS03 alone injected group (group 4) do not show monocyte number increase and did not promote Aβ uptake even after 4 injections. The contribution of AS01B in the increased antibody-mediated phagocytosis was significant since all groups with antigen that did not contain AS01B showed a lower clearance than those with AS01B (FIG. 32B), despite an antibody response of the same magnitude in the different groups (FIG. 32C). Moreover, the AS01B group that did not contain antigen only modestly stimulate phagocytosis vs. the group AS01B+antigen, which confirms the importance of the presence of anti Aβ antibody induction in this synergistic phagocytosis process.

In conclusion, this in vivo methodology developed herein shows that the Aβ uptake is promoted during or after the induction of the polyclonal Aβ-specific antibody response. More, that phenomenon is synergised with adjuvant containing formulations such as AS01B or CRX601, suggesting the presence in the peripheral blood of a mode of action that is anti Aβ antibody-mediated phagocytosis.

### Immunization Schedule:

| Group | Imm day 0 | Imm day 14 | Imm day 21 | Imm day 28 | Imm day 29 |
|---|---|---|---|---|---|
| 1 | Aβ1-6-CRM + AS03 | Aβ1-6-CRM + AS03 | Aβ1-6-CRM + AS03 | AS03 alone | Aβ1-42Hylite488 |
| 2 | Aβ1-6-CRM + AS03 | Aβ1-6-CRM + AS03 | Aβ1-6-CRM + AS03 | AS01B alone | Aβ1-42Hylite488 |
| 3 | Aβ1-6-CRM + AS03 | Aβ1-6-CRM + AS03 | Aβ1-6-CRM + AS03 | CRX601 + AS03 | Aβ1-42Hylite488 |
| 4 | AS03 alone | AS03 alone | AS03 alone | AS01B alone | Aβ1-42Hylite488 |
| 5 | AS03 alone | AS03 alone | AS03 alone | CRX601 + AS03 | Aβ1-42Hylite488 |
| 6 | Aβ1-6-CRM + AS01B | Aβ1-6-CRM + AS01B | Aβ1-6-CRM + AS01B | Aβ1-6-CRM + AS03 | Aβ1-42Hylite488 |
| 7 | Aβ1-6-CRM + AS01B | Aβ1-6-CRM + AS01B | Aβ1-6-CRM + AS01B | AS01B alone | Aβ1-42Hylite488 |
| 8 | Aβ1-6-CRM + AS01B | Aβ1-6-CRM + AS01B | Aβ1-6-CRM + AS01B | Aβ1-6-CRM + AS01B | Aβ1-42Hylite488 |
| 9 | AS01B alone | AS01B alone | AS01B alone | AS01B alone | Aβ1-42Hylite488 |
| 10 | AS03 | AS03 | AS03 | AS03 | Aβ1-42Hylite488 |

### Method:

C57BL/6 female mice were purchased from Charles River laboratories (St-Constant, Qc, Canada). Mice were immunized at day 0, 14, 21 and 28 using the intramuscular route as described herein in the FIG. 32 with different immunomodulators (AS03 or AS01B or CRX601/AS03). The AS03 and AS01B mouse dose (=1:10 human dose) have been described previously. 2 μg per mouse was used for CRX601 and was combined with the mouse dose of AS03 before injection using the intramuscular route. At day 29, 24 hr later following the last injection, 5 μg per mouse of fluorescent HiLyte Fluor™ 488-labeled Amyloid β 1-42 (Anaspec, Fremont, Calif.) was injected in the tail vein. 2 hrs later, peripheral blood was drawn from immunized C57BL/6 mice via cardiac puncture with lithium-heparin as anticoagulant, Plasma of pooled blood was decanted and saved for measurement of antibody titers by ELISA. Red blood cell lysis was performed twice with Ammonium Chloride-based Buffer (Sigma, Steinheim, Germany) and cells were counted with the EasyCount™ System (Immunicon).

The flow cytometry (FACS) experiment is performed as the following: 500,000 cells were aliquoted in a 96-well plate, washed once, incubated for 10 min. on ice in the presence of Rat anti-Mouse CD16/CD32 (clone 2.4G2—BD Fc Block™) and further stained for 30 min. with a combination of the following directly conjugated antibodies at their pre-determined optimal concentration: PE-Hamster anti-Mouse CD3 (clone 145-2C11), Rat anti-Mouse CD45R/B220 (clone RA3-6B2), Rat anti-Mouse Ly-6G (clone 1A8), Mouse anti-Mouse NK1.1 (clone PK136), APC-conjugated Rat anti-Mouse CD11b (clone M1/70), PE-Cy7-conjugated Hamster anti-Mouse CD11c (clone HL3), (all from BD PharMingen). Cells were finally washed twice and fixed for 15 min. with a 2% paraformaldehyde solution in PBS. FMOs controls were always included in the assays. Samples were acquired on a flow cytometer (BD FACSCanto II) and data analyzed with the FACSDiva software (BD Biosciences). Monocytes were identified and counted by their Side/Forward scatter properties, excluding debris and gated as CD3-/CD45R/B220-/Ly-6G-/NK1.1-(Lineage-)/CD11 b+ cells. Aβ uptake was assessed by reporting the percentage and Mean Fluorescence Intensity (GeoMean) of positive HiLyte Fluor™ 488 Aβ1-42 cells among gated monocytes or total live cells.

### Example 28

### Cytokine Measurement

While an excessive pro-inflammatory response is potentially harmful for its host, many lines of evidence highlight the importance of a fine-tuned innate immune response to fight off AD (Wyss-Coray, T. Nat. Med. 12, 1005-1015 (2006)).

It was shown that overexpression of IL-1β, TNF-α, IL-6 and IFN-γ were all reducing Aβ deposition in AD mice models (Chakrabarty et al. FASEB J. 24, 548-559 (2010); Chakrabarty et al., Mol Neurodegener 6, 16 (2011); Shaftel et al. J. Clin. Invest. 117, 1595-1604 (2007). Chakrabarty et al. J. Immunol. 184, 5333-5343 (2010).

Strongest activators of microglia are the Toll-like receptor 4 agonists such as lipopolysaccharides from E. coli (LPS). LPS provokes a rapid and strong innate activation of brain cells such as microglia and their precursors in the peripheral blood such as monocytes that come from bone marrow myeloid cells (Yong, V. W. & Rivest, S. Neuron 64, 55-60 (2009)).

However, LPS from E. coli, Salmonella and few others gram negative bacteria are strong endotoxins, are toxic and has been shown to exacerbate pre-existing neuropathology in mice when injected at the peripheral blood. LPS could not be used at clinical level because of their too high toxicity. Therefore, to avoid those detrimental effects, we evaluated 3D-MPL and compared it to the E. coli LPS in term of the innate cytokine profile (data not shown). 3D MPL delivered by the intraperitoneal (i.p.) route promoted an attenuated cytokine profile compared to LPS. This suggests that MPL might be better suited to induce the activation of phagocytic cells without provoking too much inflammatory response as LPS could do. Similarly, AS01B injected by the intramuscular route showed even more attenuated to similar biological activity on the cytokine production as 3DMPL for the i.p. route (data not shown).

### Methods:

### Cytokine Measurement:

Mice (n=5) received 50 μl of adjuvant formulations by i.m. injection for the PBS, AS03, AS01B and AS04D. PBS or LPS or MPL were injected using the intraperitoneal route and sera were collected at 2 and 6 h time points. Whole blood was collected by cardiac puncture from mice and centrifuged on a vacutainer blood collection tube containing gel for serum separation. Serum samples were cleared by centrifugation and stored at −80° C. until analysis. Protein levels in the cleared sera samples were measured by cytokine-specific beads (Millipore, USA) using the Luminex™ platform.

AS04D (MPL+Aluminium hydroxide): Typical compositions include 3D-MPL at a concentration of 100 μg/ml and Aluminium hydroxide at 1 mg/ml. It is administered 5 μg of 3D-MPL and 50 μg of Aluminium hydroxyde per dose. The 3D-MPL is adsorbed at least 1 hr before the injection onto the aluminum hydroxyde solution.

### Results:

### MPL Induces a Low Inflammatory Response in Mice

To determine whether the low inflammatory response observed in the previous in vitro experiments can be reproduced in vivo, we measured several cytokines and chemokines in the sera of C57BL/6 mice 2 and 6 h following a single intraperitoneal injection of either MPL or LPS. We found that most of the cytokine and chemokine levels were increased in MPL-injected mice, but these levels were substantially lower than those of LPS-treated animals (data not shown). The levels of TNF-α and IL-6 were very high 2 h post LPS injection while a modest increase was observed in MPL-injected mice but it was essentially abolished after 6 h. Noteworthy, IL-1β is not increased in response to MPL in contrast to LPS treatment. Interestingly, 2 h after the injection, the chemokines which are more related to monocytes and microglia activation such as CXCL-1 and CCL2 were modulated respectively to similar or higher levels in MPL-treated mice compared to the LPS group.

### Example 29

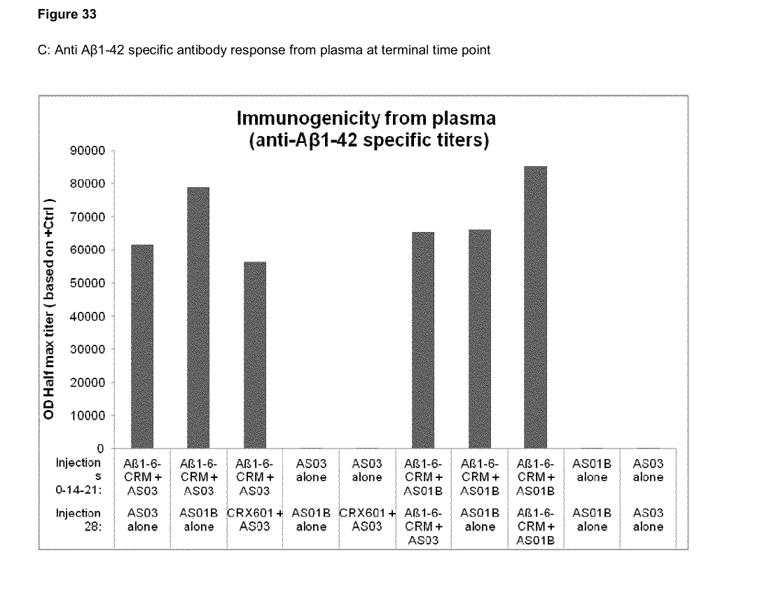

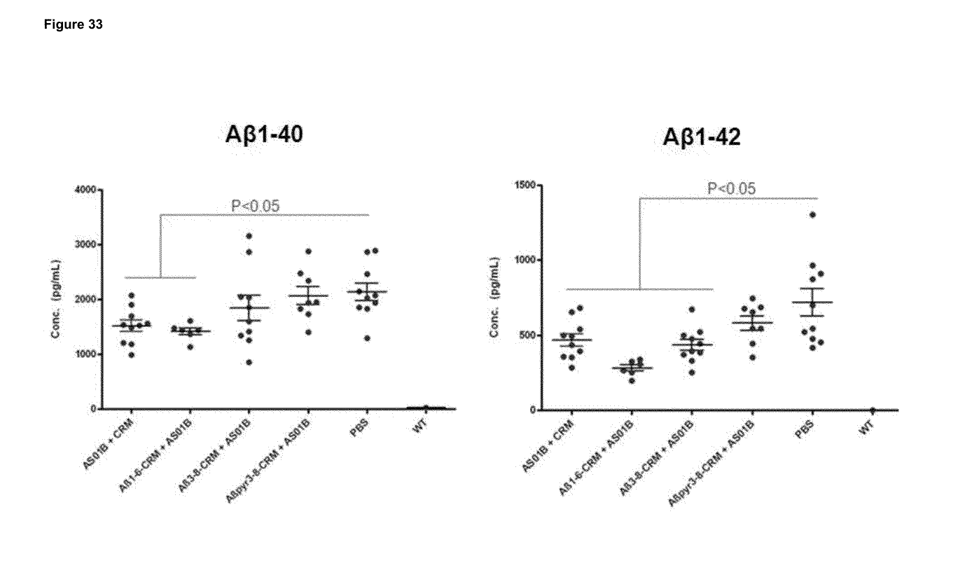

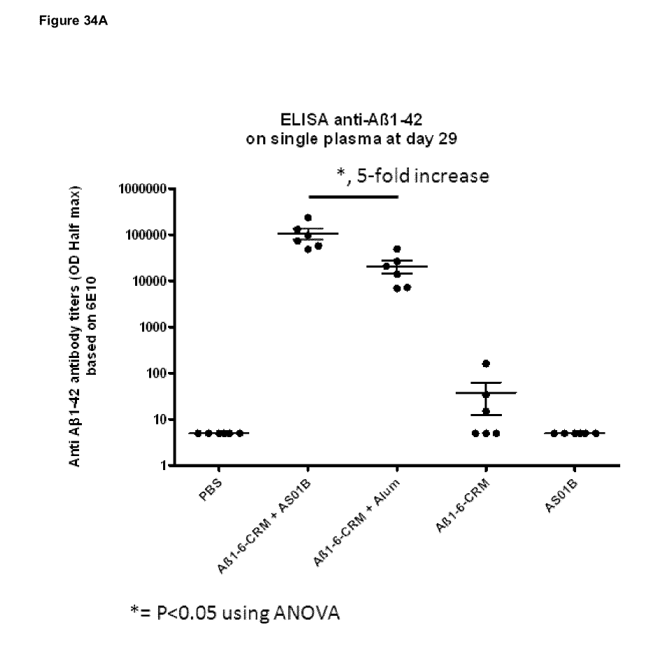

### FIG. 33

The experiment described in Example 23 had two aims: first to evaluate the impact of TLR-containing adjuvant (such as AS01B used herein) on amyloid accumulation within the blood cells, and second to evaluate the effect within the brain of the same treated mice. To measure whether AS01B+CRM197 with or without Aβ conjugated peptides affect the soluble Aβ level in the brain after a long treatment schedule over one year, Aβ was measured in the brain of Alzheimer's-like pathology mouse model (TASTPM) using the method as previously described (Englund H et al., J. Neurochem 103, 334-45 (2007)). The TASTPM model has been previously described to mount a rapid amyloid accumulation starting at 3 months of age (Howlett D R et all, Histol Histopathol. 23, 67-76 (2008)). Therefore, we performed immunization starting at that early time point to allow a prophylactic treatment.

The results showed that a long prophylactic treatment (9 mt treatment) in which we began vaccination with native Aβ peptides before the onset of Aβ deposition showed the feasibility to down-modulate the soluble amyloid (Aβ40 and Aβ42) in a mouse model containing APP and PS1 mutations (TASTPM mouse model). The Aβ1-6CRM+AS01B group showed the lowest Aβ40 and Aβ42 level amongst all treated group compared to negative control (PBS). Interestingly, the 9 mt treatment using CRM+AS01B also exhibit lower amount of soluble Aβ40 in the brain. Those results obtained from the brain extract are in line with the result obtained from the level of Aβ+positive monocytes in the peripheral blood (FIG. 27). Specially the Aβ1-6CRM+AS01B, which was low for Aβ+positive monocytes was also low for the soluble Aβ40 and Aβ42 in the brain, suggesting that the Aβ clearance occurred in the blood and in the brain in those Aβ1-6CRM+AS01B treated animal compared to non-treated animals (PBS).

### Example 30

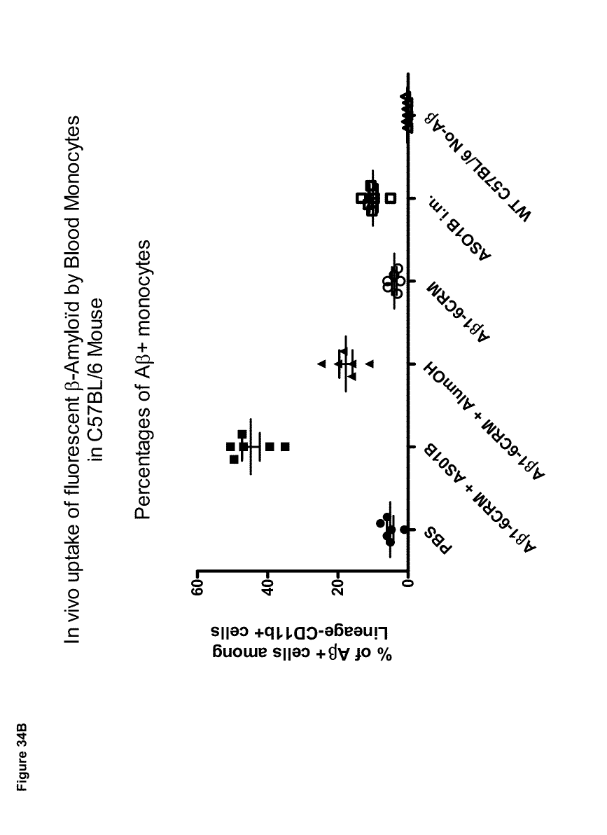

### FIG. 34

To better compare AS01B (MPL+QS21 in liposome) to the benchmark adjuvant aluminium hydroxide in combination with the same amyloid conjugated peptide (Aβ1-6CRM) we performed an experiment in which the Aβ specific titer (FIG. 34A) and Aβ uptake capacity (FIG. 34B) was measured. In brief, C57BL/6 mice were immunized with the product listed herein using the intramuscular route (50 μl per animal) at day 0-14-21-28. At day 29, sera were collected in each individual mouse (n=6 mice per group). The dose of peptide was 1 μg of Aβ1-6 peptide (DAEFRHC). The content of CRM197 in our formulations having 1 μg of Aβ1-6 peptide is 5 μg of CRM197 protein. More details are as the following:

| Group | Antigen | Carrier | Adjuvant | Other details |
|---|---|---|---|---|
| 1 | — | — | — | PBS alone |
| 2 | Aβ1-6 | CRM197 | AS01B | 5 μg QS21 in DOPC + |
| | | | | cholesterol liposome, |
| | | | | 5 μg MPL per mouse |
| 3 | Aβ1-6 | CRM197 | Alum | 50 μg AlumOH per mouse |
| | | | hydroxide | |
| 4 | Aβ1-6 | CRM197 | — | |
| 5 | — | — | AS01B | 5 μg QS21 in liposome |
| | | | | (DOPC + cholesterol), |
| | | | | 5 μg MPL per mouse |

The results described in FIG. 34A show that immunization with AS01B+Aβ1-6CRM triggers the highest Aβ1-42 specific immunogenicity. The comparison with the aluminium hydroxide group shows that AS01B+Aβ1-6CRM vaccine triggers a 5 fold higher antibody titer compared to aluminium hydroxide+Aβ1-6CRM vaccine. The negative controls herein such as PBS and AS01B group alone did not trigger Aβ1-42 specific antibody response as expected because no antigen were used in these formulations. Aβ1-6CRM alone induces a very weak to undetectable Aβ1-42 specific antibody response, implying that vaccine adjuvants are also necessary to induce the appropriate immunogenicity specific for amyloid.

Furthermore, to evaluate whether the same animals possess functional antibodies to target the uptake of Aβ, we performed an in vivo phagocytosis using HiLyte Fluor Aβ injected passively in the blood stream for a period of 2 hr as described previously. The results in FIG. 34B show the requirement to have both components, AS01B and the antigen, to induce a robust capture and clearance of peripheral Abeta by the peripheral CD11b+ monocytes in vivo compared to Alum+Ag, AS01B alone or Ag alone.

1. A method for stimulating an immune response against an antigen in an individual, the method comprising delivering to an individual a TLR agonist and an antibody which binds to that antigen.

2-4. (canceled)

5. A method according to, claim 1 wherein the TLR agonist is delivered at the same time as the antibody, less than 48 hours before the antibody or after the antibody.

6. A method, TLR agonist and an antibody, or use according to claim 5 wherein:
(a) the TLR agonist is delivered multiple times after delivery of the antibody; or
(b) the antibody is delivered multiple times before single or multiple delivery of the TLR agonist.

7. A method, TLR agonist and an antibody, or use according to claim 1 wherein the antibody is an antibody specific for β-amyloid or a fragment thereof.

8. A method according to claim 7 wherein the antibody specific for β-amyloid is selected from 6E10, 2E7, 6F6 or a humanised variant thereof such as H2L1 or H51L21.

9. A method according to claim 1 wherein the TLR agonist comprises 3D MPL or an AGP.

10. A method according to claim 1 wherein the antigen is an antigen associated with Alzheimer's disease, wet AMD, dry AMD, geographic atrophy, glaucoma or amyloid beta dependent cataract formation.

11. A composition for treatment or prevention of Alzheimer's disease, or for stimulating phagocytosis of beta amyloid, the composition comprising a TLR agonist and an antibody.

12. A composition according to claim 11 wherein:
the TLR agonist comprises 3D MPL or an AGP, and the antibody is specific for amyloid beta.

13. (canceled)

14. An assay for phagocytosis of beta amyloid, the method comprising incubation of lineage CD11 b+ monocytes with a labeled amyloid beta polypeptide and detection of the uptake of the labeled amyloid beta into the cells.

15. (canceled)


SEQUENCE LISTING (12 sequences)

SEQ ID NO: 1 - protein, 6 aa, Homo sapiens
DAEFRH

SEQ ID NO: 2 - protein, 6 aa, Homo sapiens
AEFRHD

SEQ ID NO: 3 - protein, 6 aa, homo sapiens
EFRHDS

SEQ ID NO: 4 - protein, 6 aa, Homo Sapiens
EFRHDS

SEQ ID NO: 5 - protein, 6 aa, Homo Sapiens
EVHHQK

SEQ ID NO: 6 - protein, 21 aa, Artificial Sequence
  Random Peptide
TYLIHVHIITIYHISIYYIVC

SEQ ID NO: 7 - protein, 20 aa, Artificial Sequence
  Random Peptide 3A
TYLIHVHIITIYHISIYYIV

SEQ ID NO: 8 - protein, 18 aa, Neisseria meningitidis
SQEPAAPAAEATPAAEAP

SEQ ID NO: 9 - protein, 24 aa, Artificial Sequence
  Pam3CysLip-Abeta1-6 fusion
SQEPAAPAAEATPAAEAPDAEFRH

SEQ ID NO: 10 - protein, 7 aa, Homo Sapiens
DMPVDPD

SEQ ID NO: 11 - protein, 42 aa, Homo Sapiens [sequence omitted]

SEQ ID NO: 12 - DNA, 24 bp, Artificial Sequence
  synthetic single-stranded phosphorothioate oligodeoxy-nucleotide
tcgtcgttttgtcgttttgtcgtt